(19) United States
(12) Patent Application Publication
(10) Pub. No.: US 2006/0008803 A1
(43) Pub. Date: Jan. 12, 2006

(54) Identification of tissue/cell specific marker genes and use thereof

(76) Inventors: Andreas Brunner, Oberembrach (CH); Rupert Hagg, Bassesdorf (CH); Roberto Tommasini, Uster (CH)

Correspondence Address: LADAS & PARRY, 26 WEST 61ST STREET, NEW YORK, NY, 10023, US

(21) Appl. No.: 10/517,756

(22) PCT Filed: Jun. 12, 2003
(86) PCT No.: PCT/CH03/00379

§ 371 Date: Aug. 2, 2005

Related U.S. Application Data
(60) Provisional application No. 60/388,994, filed on Jun. 14, 2002.

(51) Int. Cl. C12Q 1/68

(52) U.S. Cl. 435/6

(57) ABSTRACT

A cartilage array comprises a plurality of different polynucleotide probe spots stably associated with a solid surface of a carrier, whereby each of said spots is made of a unique polynucleotide that corresponds to one specific cartilage marker gene. Said specific cartilage marker genes preferably are at least in part selected from a group of 467 genes that could be shown to be cartilage related.

### CROSS REFERENCES TO RELATED APPLICATIONS

This application claims the priority of U.S. provisional patent application 60/388,994, filed Jun. 14, 2002, the disclosure of which is incorporated herein by reference in its entirety.

### FIELD OF THE INVENTION

The present invention relates to a method for the identification of tissue cell specific marker genes, a method for the determination of a disease state or developmental status of cells/tissue as well as to gene expression profiling of cartilage tissue. More specifically, the invention relates to microarrays containing a plurality of selected human chondrocyte specific sequences and their use for classification of cartilage donor tissue or generation of characteristic gene expression profiles of in vitro chondrocyte cultures. Such DNA arrays find use as a standard tool of molecular biology research and clinical diagnostics for all cartilaginous or related tissues.

### BACKGROUND OF THE INVENTION

Limitation on Current Microarray Technologies

DNA array technology, also known as biochip or microarray technology, is currently revolutionizing modern biology. In this technology, a biological sample is applied to a glass slide or chip covered with an array of immobilized DNA probes. Sample nucleic acid complementary to specific probes on the array hybridizes and can be detected with high sensitively with automated, computerized detectors. In this manner, hundreds to thousands of different individual hybridization experiments can be performed simultaneously. This allows assays of enormous complexity to be carried out—for example, an analysis of the entire gene expression profile of a cancer cell—with simplicity unimaginable only a few years ago. As a consequence many patents as well as scientific publications have accumulated during the last years. U.S. Pat. No. 6,194,158 discloses characteristic genes and gene expression useful in screening for, diagnosis of, monitoring of, and therapeutic treatment of brain cancer. U.S. Pat. No. 6,218,122 discloses methods for determining or monitoring the progression of disease states or the efficacy of therapeutic regimens within human patients. U.S. Pat. No. 6,077,673 discloses mouse arrays having a plurality of probe polynucleotides corresponding to a key mouse gene for expression analysis of critical mouse genes. A list of representative scientific papers dealing with monitoring the expression level of a large number of transcripts within a cell at any time are as follows: Schena et al., 1995, Quantitative monitoring of gene expression patterns with a complementary DNA-microarray, Science 270: 467-470; Lockhart et al., 1996, Expression monitoring by hybridization to high-density oligonucleotide arrays, Nature Biotechnology 14:1675-1680; Blanchard et al., 1996, Sequence to array: Probing the genome's secrets, Nature Biotechnology 14:1649. Qi et al., 2003, Identification of genes responsible for osteoblast differentiation from human mesodermal progenitor cells PNAS 18; 100 (6):3305-10. While this list of scientific papers and patents reflects without any doubt the great potential of microarrays, there are a couple of yet unsolved problems that are more and more discussed among the scientific community. Especially, these problems are data overflow, representative sample collection, RNA processing and inappropriate data analysis. It is even suspected that within next five years, many of conclusions drawn from published data will be revised or refuted. Thus there remains a real and unmet need for advanced microarray solutions, targeted to specific tissues above all with respect to simplification and substantiation of the process of data generation and data handling. With respect to this issue the disclosed invention has made considerable contribution in the cartilage area with a cartilage-specific microarray containing a manageable number of cartilage relevant genes.

Limitation on the Number of Cartilage Relevant Genes

Until today the number of cartilage-relevant genes (genes that have been associated a potential functional role on cartilage biology, homeostasis or pathology) is very limited. Approximately, 100-200 genes have been described in the literature in any relationship to cartilage tissue. While existing publications e.g. Heller et al PNAS, 94; 2150-2155; 1997 have described analysis of inflammatory diseases of cartilage and Sekiya et al PNAS 99; 43974402; 2001 cartilage formation from stem cells with microarrays, a comprehensive analysis and determination of characteristic gene expression profiles for 2D, 3D, fetal, adult and pathological chondrocytes cell cultures cultivated under different conditions has not been performed up to now. While in patent WO01/24833 A2 a few markers have been determined that are associated with chondorcytes and their phenotype stability, it will not be possible to perform a detailed gene expression analysis and to define specific fingerprints. Therefore the possibility of characterizing culture conditions or cartilage tissue samples can not be thoroughly addressed.

Completion of the human genome first project draft on 2000 has revealed that the human genome comprises ˜30000-35000 human genes. Estimates show that the number and type of active genes vary significantly between different tissues and may increase up to a couple of 10000 for complex tissues, e.g. brain. As a consequence, many genes albeit fully sequenced may have yet not been, disclosed to be functionally up- or down regulated in cartilage or cartilage derived cells. The inventive approach described herein has made possible to up to now disclose a total of 467 known and additional genes being differentially expressed in a significant and objective manner within chondrocytes or chondrogenic cells.

By means of the already known and additionally found to be cartilage related genes, a strategy to best address and represent chondrocytes cultured under different conditions has been developed in the scope of the present invention.

### SUMMARY OF THE INVENTION

In a first aspect the present invention relates to a method for the identification of tissue/cell specific marker genes comprising
a) taking tissue and/or cells of at least one developmental stage and/or at least one disease state, and/or
cultivating said tissue and/or cells in vitro under at least one culture condition,
b) determination of gene expression profiles of said tissue/cells and/or in vitro cultivated tissue/cells and
c) identification of specific marker genes by bioinformatic analysis of said gene expression profiles.

In particular, the first aspect relates to a method for the identification of tissue/cell specific marker genes comprising cultivating tissue/cells of different developmental stages and/or health conditions in vitro under different culture conditions, determination of gene expression profiles of said in vitro cultivated cartilage tissue and identification of specific marker genes by bioinformatic analysis of said gene expression profiles.

In a preferred embodiment said tissue is selected from the group consisting of fetal tissue, adolescent tissue, adult tissue, healthy tissue, pathological tissue, progenitor cells such as stem cells or cells derived from the same precursor lineage. Preferred culture conditions are 2D and 3D in vitro cultures and the gene expression profiles are preferably determined by means of a micro-array. The bioinformatic analysis of said gene expression profiles is preferably done by cluster software such as e.g cluster analysis.

In a preferred embodiment said tissue is cartilage.

A second aspect of the present invention relates to a method for the determination of a disease state or developmental status of cells/tissue or the physiological potential of cells/tissue. Said method comprises establishing a profile of cellular constituents, preferably a gene expression profile, of said cells or tissue, comparison of said resulting gene expression profile with gene expression profiles characteristic for a particular status or physiological potential of the examined cells or tissue.

Said method can e.g be used to assess the redifferentiation potential of cells or tissue, the assessment of the quality of tissue biopsies for diagnostic and prognostic purposes regarding in vitro tissue engineering applications, the assessment of the quality of in vitro produced cells such as e.g. mesenchymal cells, stem cells or embryonic cells or of in vitro produced tissue for therapeutical applications and for determining the effect of one or more growth factors, media compositions or drugs on cells or tissue. Based on said method it is e.g. possible to set up different in vitro culture conditions for cells/tissue allowing the cultivation of cells/tissue which retain their potential for differentiation.

In a preferred embodiment said cells or tissue is cartilage tissue or chondrocytes and the array comprises polynucleotide probes of tissue specific marker genes.

In a further preferred embodiment said profile is a gene expression profile which is determined by means of a micro-array.

A further object of the present invention is a method for the determination of characteristic profits for clinical use comprising correlating the patient data of the biopsy donor with the gene expression profile of said biopsy cells/tissue. Preferably said gene expression profile has been determined according to the above disclosed method. The resulting profiles of said method are suitable tools in the clinic allowing an evaluation of further treatments of a patient.

The present invention provides characteristic gene expression profiles experimentally determined by using cartilaginous tissues as from individual human donors of various ages (fetal, adolescent, adult) and health conditions (healthy and arthritic) or cells thereof cultivated under different in vitro culture conditions (2D and 3D in vitro cultures, time follow ups). From these different gene expression profiles a set of hitherto 467 markers has been deduced that can be used to design and produce a cartilage specific microarray for commercial applications in the field of R&D, such as culture media development, drug screening etc., but also for clinical applications.

Gene expression analysis performed with such microarrays and the corresponding analytical procedure thereof can be used to assess quality control of human donor cartilage, e.g. biopsy and therefore optimization of any downstream tissue engineering process, for diagnostic evaluation of the patient and its candidate treatment methods, to ensure a cost-optimized procedure, to investigate and assess all kind of 2D- and 3D in vitro cultures performed with human chondrocytes or chondrogenic cells, e.g. stem cells, to screen all kind of drugs, e.g. hormones, growth factors within the above mentioned in vitro cultures regarding a potential beneficial effect and quality assessment of in vitro produced tissue performed by tissue engineered procedures.

In a further aspect the present invention provides a cartilage array comprising a plurality of different polynucleotide probe spots stably associated with a solid surface of a carrier, whereby each of said spots is made of a unique polynucleotide that corresponds to one specific cartilage marker gene.

A preferred cartilage array of the present invention comprises at least two spots that have different nucleotide sequences but of the same cartilage marker gene, more preferably at least 10 spots indicative for one tissue or cell status, whereby said at least 10 spots can be selected from different sequences of one gene or from different genes or a combination thereof.

In a preferred embodiment said polynucleotides of the array do not cross hybridize under stringent conditions with each other.

In a preferred embodiment of the present invention the cartilage array comprises spots that are indicative for at least two tissue or cell status, preferably 3.

A further preferred inventive cartilage array is an array wherein at least part of the cartilage marker genes are selected from the 467 genes listed in the description, preferably at least 10%, more preferably at least 50%, most preferably about 100%.

A further preferred inventive cartilage array is an array wherein at least part of the cartilage marker genes are selected from a subgroup of the 467 genes listed in the description, wherein said subgroup consists of the most tissue specific 200 genes.

In another preferred embodiment the status is selected from biopsies and/or 2D cultures and/or 3D cultures of healthy adult, healthy fetal/infant, undesired adult, undesired fetal/infant or progenitor cells like e.g. stem cells or cells derived from the same precursor lineage.

In a further preferred embodiment of the present invention the polynucleotide probes of the cartilage array have a length of at least 10 nucleotides, preferably at least 20 nucleotides. The probes can also have a length of 30 nucleotides, 50 nucleotides or 70 nucleotides. It is as well possible to use PCR derived products produced from cDNA clones.

In a preferred embodiment the carrier of the inventive cartilage array is attached to coated glass, nylon or any other material.

A further object of the present invention is a kit for use in a hybridization assay, wherein said kit comprises a cartilage array of the present invention. In a preferred embodiment said kit comprises reagents for generating a labelled target polynucleotide sample, a hybridization buffer and a wash medium.

### DESCRIPTION OF THE FIGURES

The present invention will be further understood from the following description with reference to the tables and figures where:

Tab. I shows the determined number of all genes in the corresponding SOM analysis being differentially expressed according to microarray analyses of a variety of in vitro chondrocyte cultures according to predefined criteria. From these data sets specific expression profiles can be deduced that are characteristic for different cell culture conditions.

Tab. II shows the extracted and reviewed genes deduced from Tab I in order to have only single entry numbers. Since most of these genes have never been described in any relationship to cartilage, they can be considered as novel cartilage marker (positive/negative markers) or key cartilage genes.

Tab III shows a subset of marker genes form Tab. II that has been used for the production of a micro-array. Included is a subset from Tab II and genes known from the literature.

Tab IV shows the results of the analysis of the 467 cartilage specific marker genes.

Tab V shows the samples used in Examples 1, 2 and 3. Human chondrocytes isolated from 4 different donors were proliferated and kept in 3D-like pellet culture for 7 and 14 days resulting in a total number of 12 samples.

FIG. 1 shows a classical result from an analysis performed with self-organizing-maps. This software clusters all genes together in sub clusters that show a similar expression profile. The number of marker genes for the corresponding analysis e.g. 2D vs. 3D cultures (see also Tab I) corresponds to the total number of genes in the sub clusters.

FIG. 2 shows an example of a graphical presentation of a cluster analysis and viewed by the software treeview. This shows how cells from different origin and potential for in vitro cartilage formation are related to each other and allow a clearer classification of the cell sources. Fetal cells clearly produce different gene clusters compared to adult chondrocytes, while failures are characterized by other gene clusters. Furthermore 3D cell cultures analyzed in a time dependent manner from different donors can be distinguished among each other and gene expression profiles will be grouped accordingly.

FIG. 3: SOM analysis of all culture conditions and samples described in Example 2 and in Tab V.

FIG. 4: SOM analysis for proliferated chondrocytes (t0) only, for the 4 donors. Gene expression pattern corresponding to donor 2 (the second spot from left hand side in every cluster) behaves different in most clusters.

FIG. 5: SOM analysis of chondrocytes kept in 3D culture condition for 7 days (t7). Gene expression pattern from donor 3 (the third spot from left hand side in every cluster) is different for example in clusters c2 and c5.

FIG. 6 shows self organized maps (SOM) of chondrocytes from same patients of FIGS. 4 and 5 kept under 3D culture condition for 14 days (t14).

FIG. 7: cluster analysis of all culture conditions and samples described in Example 2 and in Tab V. This figure shows a subset of 88 hierarchical clustered genes (rows) and samples (columns) demonstrating similar gene expression behavior of chondrocytes under different culture conditions. For example proliferated cells (#1, #2, #4, #5, #7, #8, #10, #11) can easily be discriminated from cells kept in 3D-like pellet culture for 14 days (3#, 6#, 9#, 12#).

FIG. 8: cluster analysis of human aortic fibroblasts vs. chondrocytes. This figure shows a subset of selected clusters of human aortic fibroblasts cells compared to human chondrocytes both kept in 3D pellet cultures for 14 days. The dendrogram in the upper part of the figure shows the ability of CART-CHIP™ 300 microarray described in this invention to discriminate between different cell lines.

FIG. 9: cluster analysis of Interleukin-1 treated vs. untreated human chondrocytes. This figure demonstrates a subset of representative gene clusters allowing differentiation between cells treated with Interleukin-1 from untreated cells both kept in 3D pellet cultures as well as for proliferated cells.

### DETAILED DESCRIPTION OF THE INVENTION

Definitions

2D cultures as used in the scope of the present invention are anchorage dependent chondrocyte cultures cultivated on plastic culture devices.

3D cultures as used in the scope of the present invention are chondrocytes cultured in a three dimensional environment, namely either a) scaffold-free, such as small high density pellet cultures (0.25-3.0*10⁶ cells) or as high density cultures using 50*10⁶ cells/ml or aliquots thereof; or b) by using a synthetic scaffold such as PGA, PLA, or mixtures thereof or biological substances such as agarose, alginate, chitosan or collagen.

failures as used in the scope of the present invention are chondrocytes cultured in a three dimensional environment that are not able to synthesize new extracellular matrix thereby compromising the production of new living tissue engineered cartilage equivalents.

gene expression profile as used in the scope of the present invention is a profile of genes that are up or down regulated according to different cell conditions.

fingerprint as used in the scope of the present invention refers to a gene expression profile characteristic for a cellular status.

tissue or cell status as used in the scope of the present invention refers to a tissue or cells therof having a certain metabolic or activity status.

new extracellular matrix as used in the scope of the present invention designates living cartilage-like tissue.

micro-array as used in the scope of the present invention is used in its original scope that encompasses embodiments today sometimes refused to as “macro-arrays”.

The Invention

The present invention provides cartilage-specific gene arrays as well as methods for their use. In the subject cartilage arrays, a plurality of polynucleotide probe spots are stably associated with the surface of a solid carrier, preferably a surface of a microscope glass slide. Each different polynucleotide probe spot is made of a unique polynucleotide that corresponds to a key cartilage gene of interest. Thus, the subject arrays find particular use in gene expression assays of key cartilage genes. In further describing the subject of the invention, the cartilage specific microarrays are first discussed, followed by a review of representative applications in which the subject arrays may be employed.

Arrays of the Subject Invention-General Description

Selection of Novel Key Cartilage Specific Genes:

A critical feature of the subject arrays is that all of the probe polynucleotide spots of the array correspond to human key cartilage genes that have been found through unique selection processes and criteria. As a result of said processes, up to now 467 different key human cartilage genes that are under tight transcriptional role have been discovered, some of them being not described before in any relationship to cartilage. In more detail, different microarray analyses were performed by using cartilaginous tissues as from individual human donors of various ages (fetal, adolescent, adult) and health conditions (healthy and arthritic) or cells thereof cultivated under different in vitro culture conditions (2D and 3D in vitro cultures, time follow ups). This variety of cartilage cell sources and different culture conditions was set up to grasp the highest possible number of genes differentially expressed and thus being indicative of a potential role.

It has been found that specific chondrocyte culture conditions are of great importance for the present invention that discloses a plurality of novel key cartilage genes as well as characteristic and meaningful gene expression patterns. For this reason, the strategy and criteria of the analysed in vitro human chondrocyte cultures are described in more detail. The principal experimental setup included both the cultivation of chondrocytes in an anchorage dependent condition, known as 2D cultures for expansion of cells e.g. where the passages is variable but at least more then one, as well as cultivation of chondrocytes in an anchorage independent condition, known as 3D cultures for (re-)differentiation and de novo tissue formation of cells. These are the key steps of any tissue engineering process where autologous tissue equivalents are produced. Since the cell source is either a small biopsy, a small bone marrow aspirate in case of mesenchymal stem cells or other tissue with a limited number of pre-chondrogenic cells, it is first necessary to isolate those cells in order to be able to multiply the cell number drastically. In case of a cartilage biopsy, cells are released from their surrounding extracellular matrix by collagenase digestion and then seeded onto the surface of plastic tissue culture flasks. The proliferation may take place either in the presence or absence of fetal serum combined with conventional DMEM/F12 medium. Cells can then be passaged by trypsin treatment over several rounds. As a major drawback of this necessary cell expansion, the cells loose their differentiated phenotype and assume a de-differentiated phenotype with altered gene expression. It is further known that with increasing number of passages the state of de-differentiation also advances. As a consequence, genes being transcriptionally upregulated under such artificial culture conditions are cartilage relevant in a manner being indicative of an undesired cellular status. It is also quite common to designate these genes as de-differentiation or negative markers. While healthy tissue in general has been found to re-differentiate in 3D culture after up to 4 passages in 2D cultures, tissue of undesired cellular status cultivated under usual conditions, such as usual culture media, usually does not re-differentiate in 3D culture after at most 4 passages in 2D culture.

Subculture modulated chondrocytes that do not express differentiation markers reexpress the differentiated phenotype in response to the anchorage—independence resulting from various 3D culture models, e.g. high density cultures, agarose or alginate cultures, or cultures within synthetic scaffolds such as made of polyglycolic acid (PGA), polylactic acid (PLA) or mixtures thereof. To set up three dimensional cell cultures the cells are detached after proliferation by trypsin treatment and embedded either in gel-like substances such as alginate, seeded within a porous scaffold such as PGA or cultivated as high-density cultures, only. The time for the analysis may vary and ideally addresses several time points (up to several weeks). Thus 3D in vitro chondrocyte cultures support the differentiated phenotype of chondrocytes and can be used to discover cartilage relevant genes or differentiation markers. It should be noted however, that reversibility of the de-differentiation process is dependent on the number of passages and can become irreversible or at least partially irreversible at higher passage numbers (under usual conditions at most about 4 passages). As a rule the time course of de- and re-differentiation are similar. During skeletal development, cartilage serves as a template for bone formation. Chondrocytes of fetal or infant (<1 year) or growth plate cartilage pass through different stages and exhibit several distinct phenotypes, such as resting, proliferating, and hypertrophic chondrocytes. Progression through each of these phases is accompanied by profound changes in gene expression patterns. Further, evidence has accumulated that the successful sequence of cartilage repair via tissue engineering recapitulates aspects of embryonic tissue formation. For these reasons, it is important to consider fetal and infant cartilaginous tissue. Cells isolated from human fetal/infant cartilage that are cultivated in 2D and 3D culture systems as described above are especially helpful to understand the mechanisms underlying the phenotypic instability of chondrocytes and the related gene expression patterns. These 2D and 3D culture system may then be analyzed to deduce gene expression profiles and to define marker genes that are characteristic for the (re-)differentiation process. Thus maintenance of chondrocyte-specific phenotype being crucial for normal structure and biomechanical properties of articular cartilage may be better understood and have important implications for modern therapeutic biological applications.

The above mentioned experimental setup for 2D and 3D cultures may be even expanded to compare human adult cells with human fetal/infant chondrocytic cells of age <1 year. The comparison of gene expression profiles of adult versus fetal/infant human chondrocytes during the in vitro cartilage formation process is an important aspect since marker genes associated with developmental aspects are revealed. This can be of further interest when 3D cell cultures need to be optimized for their in vitro performance for the production of new tissue by e.g. adding growth factors that are found to play a major role during the early onset of cartilage formation in vivo.

Another experimental setup found in the scope of this invention includes the in vitro culture of cells harvested from cartilaginous areas of arthritic knee joints. Osteoarthritis (OA) results from the failure of chondrocytes within the joint to maintain the balance between synthesis and degradation of extracellular matrix. OA is a multifactorial disorder in which aging, genetic, hormonal and mechanical factors are all major contributors to its onset and progression. With progressing disease state, the articular chondrocytes ability to maintain homeostasis and functionality is increasingly disappearing. As a consequence, the phenotype of osteoarthritic chondrocytes compared with normal chondrocytes exhibits remarkable changes. Gene expression profiling allows characterization of the osteoarthritic cellular phenotype, a key determinant for understanding and manipulation of osteoarthritic processes. By studying and comparing the gene expression profiles of chondrocytes harvested from pathological and healthy human cartilage areas it becomes possible to identify marker genes that are able to predict the future outcome of cell cultures used for in vitro tissue engineering applications. This also relates to the very critical question of the assessment of the quality of the starting biopsy material that is being used for downstream applications like tissue engineering. By having this important information before performing any downstream applications like e.g. proliferation and consecutive 3D in vitro tissue formation, the further steps of any process can then be adapted or even not performed at all because of inadequate quality of biopsy material. Such decision may be of high relevance when tissue-engineering processes are transferred or applied in the clinic. Gene expression profiling of chondrocytes may then be used as a diagnostic tool to allow and to choose that therapeutic approach with the most promising clinical outcome.

A further important aspect of the invention is the observation that chondrocytes derived from osteoarthritic patient material always qualify for anchorage dependent proliferation in 2D over several passages. These cells however, if subsequently induced to re-differentiate by culturing them as 3D high density pellets, do not survive over an extended time period, in most cases they die in culture by undergoing apoptosis. It is assumed that these cells, due to an altered phenotype, are not capable of producing the critical survival factors in the appropriate concentrations, above all extracellular matrix components providing intercellular spaces as they occur in native cartilage. Cells that are not suitable to be cultured within 3D high density cultures are herein referred to as “failures”. These impaired cell cultures can be used to set up representative “failure” systems, where cells from different pathological cartilage sources are harvested, proliferated and cultivated in 3D high density pellet culture systems. After each of these experimental steps, RNA can be isolated from the different cell sources and combined to create “failure pools”. These failure pools are very well suitable to identify general marker genes being indicative of the onset of osteoarthritis.

For finding cartilage relevant genes, and for determining their presence dependent on the specific cartilage type such as age, health etc., sufficient material must be generated, e.g. by 2D culturing over several passages, and optionally 3D culturing. Said material then can on be subjected to usual gene analyses, and the tissue specific genes determined. Cartillage samples are classified prior to culturing and/or after culturing to get the information needed for later interpretation of the gene expression profile.

A further experimental setup of the current inventions discloses the analysis of chondrocytes grown in 3D cultures isolated from pathological human cartilage and analyzed in a time dependent manner. This experimental set-up allows to study the apoptotic process and to further define additional dynamic and characteristic gene expression profiles, useful for deducing and further assessment of the quality of the biopsy material.

The microarray process and strategy for disclosing all the cartilage relevant genes with the above-mentioned tissues and cell culture criteria will be described in the following. An important issue of the inovative strategy used by the inventors of the present inventions is to use various microarrays containing a high number of genes comprising different functional categories preferentially by representing the whole genome. The broader the microarray regarding the coverage of the human genome the more genes associated with chondrocyte cell cultures can be determined. The chosen strategy of the inventors was not obvious to a person skilled in the art.

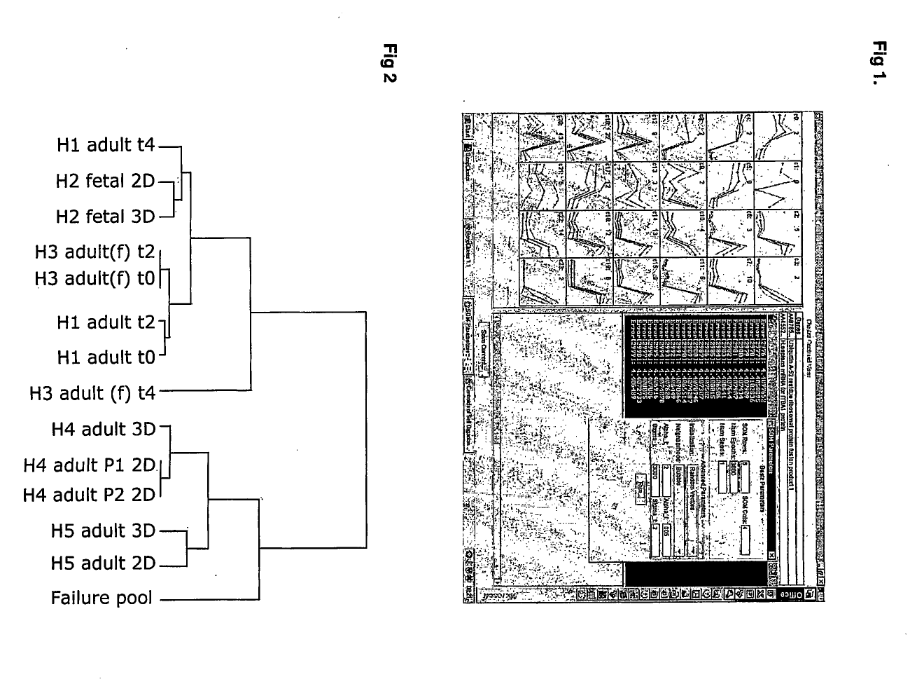

RNA isolated from the above mentioned different cell cultures conditions may be radioactive labeled with e.g. 33P or fluorescence like e.g. Cy3 and hybridized to the corresponding filters or microarrays. After hybridization each array may then be scanned and the corresponding signals measured (Tab IV). This raw data file needs then to be calibrated and normalized in a manner to create an input file for the further downstream analysis process. In principle if the data are normalized an expression profile is created. To identify the key cartilage marker genes being differentially expressed under the chosen criteria, tedious bioinformatic analysis are conducted. Corresponding cell cultures and their expression profiles are therefore compared and analyzed accordingly and the different clusters of marker genes determined by software analysis e.g. self-organizing maps (herein referred to as SOM). A representative example of a result for the comparison of different gene expression profiles from different cell culture conditions performed by SOM analysis is given in FIG. 1. By performing SOM analysis genes that are similarly expressed are clustered together in so-called sub clusters. The total amount of marker genes for one analysis corresponds to the total amount of sub clusters containing the corresponding genes. Table I in the appendix summarize the results of all the different analysis performed and encompasses all the genes determined for every set of cell culture analysis.

By performing this analytical procedure the analysis reveals several characteristic up and down regulated marker genes for different cellular culture conditions. From these marker genes characteristic expression profiles can then be deduced and used as a benchmark for the comparison or further characterization of other cell cultures.

Hence, on the one hand previously unknown cartilage—relevant genes associated with different culture conditions and on the other hand characteristic gene expression profiles (cellular fingerprints) indicative of a stage of development, a disease state or a particular selected cell culture condition are revealed. These fingerprints are part of the current invention and are of major importance for the classification and characterization of chondrocytes cultivated under different culture conditions.

Since the gene clusters from all the different analysis contain repetitive gene entries, they have been further processed so that only single entry genes are recorded (see Tab II). This 467 selected sequences are thus all key cartilage genes that are activated and thus differentially expressed according to a stage of development, a disease state or a particular selected cell culture condition and are part of the current invention. A list of all 467 genes with their Pubmed accession no. and a description is given below See also Tables II and III):

List of Table II Related Sequences:

| Pubmed | |
|---|---|
| Accesion No | Description |
| AA283693 | Human osteoclast stimulating factor mRNA, complete cds |
| AA845156 | Serine protease inhibitor, Kazal type 1 |
| R52548 | Human superoxide dismutase (SOD-1) mRNA, complete cds |
| T67128 | ARYLAMINE N-ACETYLTRANSFERASE, MONOMORPHIC |
| AA845015 | Elastase 1, pancreatic (elastase IIA) |
| AA937895 | Antigen identified by monoclonal antibodies 12E7, F21 and O13 |
| AA844998 | Pancreatic polypeptide |
| AA844818 | Amylase, alpha 2A; pancreatic |
| AA894557 | Creatine kinase B |
| AA872001 | Annexin VI (p68) |
| H09590 | Human mRNA for eukaryotic initiation factor 4AI |
| AA868278 | Testis specific protein 1 (probe H4-1 p3-1) |
| AA490855 | Acid finger protein ZNF173 |
| H05820 | Human MRL3 mRNA for ribosomal protein L3 homologue (MRL3 = mammalian |
| | ribosome L3) |
| N57766 | Agammaglobulinaemia protein-tyrosine kinase atk |
| AA873885 | Alkaline phosphatase, liver/bone/kidney |
| AA878880 | Interferon (gamma)-induced cell line; protein 10 from |
| R54818 | Human eukaryotic initiation factor 2B-epsilon mRNA, partial cds |
| AA458630 | RENIN PRECURSOR, RENAL |
| W37864 | Phosphatase and tensin homolog (mutated in multiple advanced cancers 1) |
| N63192 | Phenylethanolamine N-methyltransferase |
| R55789 | Human X11 protein mRNA, partial cds |
| R56871 | Human chromatin assembly factor-I p60 subunit mRNA, complete cds |
| AA448659 | M-PHASE INDUCER PHOSPHATASE 2 |
| AA235388 | Tropomodulin |
| W37769 | Chromogranin B (secretogranin 1) |
| AA421701 | H. sapiens mRNA for MUF1 protein |
| N81029 | Collagen, type XVIII, alpha 1 |
| AA644128 | Nuclear autoantigenic sperm protein (histone-binding) |
| N26536 | ATPase, Cu++ transporting, beta polypeptide (Wilson disease) |
| AA890663 | Human protein kinase PAK1 mRNA, complete cds |
| AA405987 | Glycerol kinase 2 (testis specific) |
| AA888182 | Ribosomal protein S4, X-linked |
| H09730 | Adenylate kinase 2 (adk2) |
| AA285155 | CDC46 HOMOLOG |
| AA873351 | Ribosomal protein L35a |
| H12320 | CAMP-RESPONSE ELEMENT BINDING PROTEIN |
| AA856556 | Ribosomal protein S28 |
| R43581 | Human guanine nucleotide-binding protein G-s, alpha subunit mRNA, partial cds |
| AA633768 | 60S RIBOSOMAL PROTEIN L24 |
| AA496880 | Ribosomal protein L5 |
| AA625632 | Ubiquitin A-52 residue ribosomal protein fusion product 1 |
| R40850 | H. sapiens mRNA for alpha-centractin |
| AA486072 | Small inducible cytokine A5 (RANTES) |
| N80129 | Metallothionein 1L |
| T67270 | UBIQUINOL-CYTOCHROME C REDUCTASE COMPLEX SUBUNIT VI REQUIRING |
| | PROTEIN |
| AA775364 | 60S RIBOSOMAL PROTEIN L30 |
| AA464743 | Ribosomal protein L21 |
| AA663983 | Triosephosphate isomerase 1 |
| AA634008 | 40S RIBOSOMAL PROTEIN S23 |
| AA683050 | 40S RIBOSOMAL PROTEIN S8 |
| AA775874 | 60S RIBOSOMAL PROTEIN L18 |
| AA029934 | Integrin, alpha V (vitronectin receptor, alpha polypeptide, antigen CD51) |
| AA872397 | GALECTIN-2 |
| AA428195 | Protein tyrosine phosphatase, non-receptor type 2 |
| AA478724 | Insulin-like growth factor binding protein 6 |
| T40541 | H. sapiens mRNA for human giant larvae homolog |
| N33214 | H. sapiens mRNA for membrane-type matrix metalloproteinase 1 |
| W69399 | Homo sapiens adenosine triphosphatase mRNA, complete cds |
| H85454 | Homo sapiens delayed-rectifier K+ channel alpha subunit (KCNS1) mRNA, complete |
| | cds |
| T71284 | Complement component 1, q subcomponent, beta polypeptide |
| N95418 | Human FK-506 binding protein homologue (FKBP38) mRNA, complete cds |
| AA430675 | Human DNA repair protein XRCC9 (XRCC9) mRNA, complete cds |
| AA682851 | Homo sapiens mRNA for ERp28 protein |
| AA427433 | PROTEIN PHOSPHATASE PP2A, 65 KD REGULATORY SUBUNIT, ALPHA |
| | ISOFORM |
| AA100296 | H. sapiens PAP mRNA |
| AA070997 | Proteasome (prosome, macropain) subunit, beta type, 6 |
| R27585 | Proteasome component C2 |
| N71628 | Spi-B transcription factor (Spi-1/PU.1 related) |
| AA464566 | Human mRNA for LDL-receptor related protein |
| AA043228 | Calponin 3, acidic |
| AA478273 | APEX nuclease (multifunctional DNA repair enzyme) |
| H05619 | Homo sapiens GDNF family receptor alpha 2 (GFRalpha2) mRNA, complete cds |
| AA405562 | Protein phosphatase 4 (formerly X), catalytic subunit |
| AA147043 | Homo sapiens CAGH1a (CAGH1) mRNA, partial cds |
| AA035384 | Homo sapiens mRNA for small subunit of cytochrome b in succinate dehydrogenase |
| | complex, complete cds |
| R60150 | Human mRNA for histidyl-tRNA synthetase (HRS) |
| N64051 | Homo sapiens Werner syndrome gene, complete cds |
| AA405748 | SPLICING FACTOR U2AF 65 KD SUBUNIT |
| AA461110 | Homo sapiens growth-arrest-specific protein (gas) mRNA, complete cds |
| AA845167 | ELASTASE IIIA PRECURSOR |
| AA443118 | Homo sapiens mRNA for CD151, complete cds |
| N92319 | Glycoprotein Ib (platelet), beta polypeptide |
| AA187148 | Core-binding factor, beta subunit |
| AA253413 | Friedreich ataxia |
| AA046701 | ATP SYNTHASE LIPID-BINDING PROTEIN P1 PRECURSOR |
| AA164562 | Homo sapiens actin-related protein Arp3 (ARP3) mRNA, complete cds |
| AA496357 | Homo sapiens SKB1Hs mRNA, complete cds |
| AA180742 | TUBULIN ALPHA-4 CHAIN |
| AA454743 | Human protease M mRNA, complete cds |
| AA437226 | Interleukin 10 receptor |
| AA458849 | Homo sapiens placental bikunin mRNA, complete cds |
| AA504891 | Crystallin, alpha B |
| AA609655 | Homo sapiens mRNA for SCP-1, complete cds |
| AA599158 | MULTIFUNCTIONAL AMINOACYL-TRNA SYNTHETASE |
| AA052932 | Homo sapiens casein kinase I gamma 2 mRNA, complete cds |
| AA789328 | Homo sapiens (clone PK2J) CDC2-related protein kinase (PISSLRE) mRNA, |
| | complete cds |
| AA129537 | Human GAP SH3 binding protein mRNA, complete cds |
| AA486209 | Low density lipoprotein-related protein-associated protein 1 (alpha-2-macroglobulin |
| | receptor-associated protein 1 |
| H39018 | H. sapiens Syt V gene (genomic and cDNA sequence) |
| AA464217 | V-akt murine thymoma viral oncogene homolog 1 |
| T95053 | Homo sapiens Rigui (RIGUI) mRNA, complete cds |
| AA454646 | LYMPHOTOXIN-BETA RECEPTOR PRECURSOR |
| AA448400 | Human plectin (PLEC1) mRNA, complete cds |
| H13691 | Major histocompatibility complex, class II, DM beta |
| AA132086 | Homo sapiens RCL (Rcl) mRNA, complete cds |
| AA488073 | Mucin 1, transmembrane |
| N40945 | H. sapiens mRNA for DRES9 protein |
| R55705 | Homo sapiens orexin receptor-1 mRNA, complete cds |
| H50114 | Homo sapiens NMDA receptor mRNA, complete cds |
| AA452841 | Human K-CI cotransporter (hKCC1) mRNA, complete cds |
| W73790 | IMMUNOGLOBULIN-RELATED 14.1 PROTEIN PRECURSOR |
| N30302 | POSSIBLE GTP-BINDING PROTEIN HSR1 |
| AA291556 | Human ras inhibitor mRNA, 3′ end |
| AA598510 | Human APRT gene for adenine phosphoribosyltransferase |
| AA453787 | Human TFIIB related factor hBRF (HBRF) mRNA, complete cds |
| H05655 | Human transcriptional activator mRNA, complete cds |
| AA419177 | INTEGRAL MEMBRANE PROTEIN E16 |
| AA458807 | Human retinal protein (HRG4) mRNA, complete cds |
| AA293218 | Cleavage stimulation factor, 3′ pre-RNA, subunit 2, 64 kD |
| W44860 | Human calmodulin mRNA, complete cds |
| AA629862 | Homo sapiens mRNA for smallest subunit of ubiquinol-cytochrome c reductase, |
| | complete cds |
| AA447674 | Homo sapiens HIV-Nef associated acyl CoA thioesterase (hNAACTE) mRNA, |
| | complete cds |
| T52484 | Nerve growth factor beta |
| AA496810 | Protein kinase C substrate 80K-H |
| AA486233 | G1 to S phase transition 1 |
| AA079775 | TYROSINE-PROTEIN KINASE CSK |
| W73889 | Tetranectin (plasminogen-binding protein) |
| R50337 | Solute carrier family 19 (folate transporter), member 1 |
| R55046 | MpV17 transgene, murine homolog, glomerulosclerosis |
| R46821 | T-COMPLEX PROTEIN 1, ALPHA SUBUNIT |
| R87763 | Human telencephalin precursor mRNA, complete cds |
| H69583 | Human BTG2 (BTG2) mRNA, complete cds |
| R56046 | Guanine nucleotide binding protein (G protein), alpha z polypeptide |
| AA922705 | Glycogen phosphorylase B (brain form) |
| AA487571 | Surfactant, pulmonary-associated protein C |
| AA402440 | Homo sapiens exportin t mRNA, complete cds |
| H29521 | ATP-binding cassette 3 |
| AA490911 | Homo sapiens drp1 mRNA, complete cds |
| AA486082 | Homo sapiens sgk gene |
| AA678065 | 2,3-bisphosphoglycerate mutase |
| R43509 | Human Gu binding protein mRNA, partial cds |
| N57553 | Adenosine receptor A2 |
| AA676955 | Aplysia ras-related homolog 12 |
| R14692 | Human Na/H antiporter (APNH1) mRNA, complete cds |
| AA488979 | Homo sapiens nucleolar protein (MSP58) mRNA, complete cds |
| AA443630 | Aldehyde dehydrogenase 8 |
| AA027840 | H. sapiens mRNA for RIT protein |
| AA456830 | Diacylglycerol kinase, alpha (80 kD) |
| AA453015 | H. sapiens L23-related mRNA |
| AA074446 | Human GTP cyclohydrolase I feedback regulatory protein gene, complete cds |
| AA027042 | DNA-DIRECTED RNA POLYMERASE II 23 KD POLYPEPTIDE |
| AA629923 | Human mRNA for pM5 protein |
| AA460830 | Homo sapiens (clone mf.18) RNA polymerase II mRNA, complete cds |
| AA454218 | Homo sapiens transcription factor SL1 mRNA, complete cds |
| AA046523 | H. sapiens mRNA for centrin gene |
| R51346 | Human eIF-2-associated p67 homolog mRNA, complete cds |
| AA029964 | Human ataxin-2 related protein mRNA, partial cds |
| AA489219 | DUTP pyrophosphatase |
| AA043133 | Solute carrier family 16 (monocarboxylic acid transporters), member 1 |
| AA812973 | Human mRNA for testis-specific TCP20, complete cds |
| AA453471 | GANGLIOSIDE GM2 ACTIVATOR PRECURSOR |
| AA284693 | Transcription factor AP-4 (activating enhancer-binding protein 4) |
| N90281 | Human B7 mRNA, complete cds |
| AA629542 | Brush-1 |
| AA679345 | Human BTK region clone ftp-3 mRNA |
| H37774 | Tuberin |
| T97181 | Platelet factor 4 |
| AA454879 | Plasminogen activator, urokinase receptor |
| AA147640 | Phosphorylase, glycogen; liver (Hers disease, glycogen storage disease type VI) |
| AA757429 | Human serotonin N-acetyltransferase mRNA, complete cds |
| AA490991 | Homo sapiens HnRNP F protein mRNA, complete cds |
| AA422058 | H. sapiens mRNA for D1075-like gene |
| N66208 | Human (ard-1) mRNA, complete cds |
| AA630776 | Human AP-3 complex delta subunit mRNA, complete cds |
| AA827287 | Human interferon-induced leucine zipper protein (IFP35) mRNA, partial cds |
| AA488084 | Superoxide dismutase 2, mitochondrial |
| R89715 | Protein kinase C, gamma |
| AA490501 | H. sapiens mRNA; UV Radiation Resistance Associated Gene |
| N32199 | Human melanoma antigen recognized by T-cells (MART-1) mRNA |
| AA434404 | DNA primase polypeptide 2A (58 kD) |
| N93686 | Aldehyde dehydrogenase 7 |
| AA292676 | Human metargidin precursor mRNA, complete cds |
| AA464417 | INTERFERON-INDUCIBLE PROTEIN 1-8U |
| AA442092 | Catenin (cadherin-associated protein), beta 1 (88 kD) |
| AA026644 | Transcription factor 3 (E2A immunoglobulin enhancer binding factors E12/E47) |
| AA481464 | Peptidylprolyl isomerase B (cyclophilin B) |
| T68859 | Alpha-2-plasmin inhibitor (alpha-2-PI) |
| AA699560 | Surfeit 1 |
| AA705069 | Human mRNA for receptor of retinoic acid |
| AA457739 | Homo sapiens putative OSP like protein mRNA, partial cds |
| H99843 | Homo sapiens mRNA for quinolinate phosphoribosyl transferase, complete cds |
| AA399410 | Signal transducer and activator of transcription 3 (acute-phase response factor) |
| AA443039 | HEAT SHOCK 70 KD PROTEIN 1 |
| AA164440 | Human autoantigen pericentriol material 1 (PCM-1) mRNA, complete cds |
| AA446453 | Human mRNA for c-myc binding protein, complete cds |
| AA280692 | Diacylglycerol kinase delta |
| AA031514 | Matrix metalloproteinase 7 (matrilysin, uterine) |
| R33154 | Msh (Drosophila) homeo box homolog 1 (formerly homeo box 7) |
| AA487452 | Human DNA fragmentation factor-45 mRNA, complete cds |
| AA400329 | Human gene for neurofilament subunit M (NF-M) |
| AA454668 | Prostaglandin-endoperoxide synthase 1 (prostaglandin G/H synthase and |
| | cyclooxygenase) |
| AA486393 | Cytokine receptor family II, member 4 |
| R52541 | unknown EST |
| AA171613 | Homo sapiens carbonic anhydrase precursor (CA 12) mRNA, complete cds |
| AA235706 | Human TATA-binding protein associated factor 30 kDa subunit (tafII30) mRNA, |
| | complete cds |
| AA668527 | Human mucosal addressin cell adhesion molecule-1 (MAdCAM-1) mRNA, complete |
| | cds |
| T54144 | Homo sapiens homolog of the Aspergillus nidulans sudD gene product mRNA, |
| | complete cds |
| R14080 | Calcium modulating ligand |
| AA609599 | Homo sapiens SSX3 (SSX3) mRNA, complete cds |
| AA489201 | H. sapiens mRNA for PHAPI2b protein |
| R08876 | Human 26S proteasome-associated pad1 homolog (POH1) mRNA, complete cds |
| H46425 | H. sapiens Pur (pur-alpha) mRNA, complete cds |
| R56149 | Human putative transmembrane protein (nma) mRNA, complete cds |
| AA454619 | Homo sapiens mRNA for Hic-5, partial cds |
| H15445 | H. sapiens mRNA for SEX gene |
| AA705225 | Myosin, light polypeptide 4, alkali; atrial, embryonic |
| AA191488 | Human high-affinity copper uptake protein (hCTR1) mRNA, complete cds |
| N64862 | Human SLP-76 associated protein mRNA, complete cds |
| R45413 | Human transmembrane 4 superfamily protein (SAS) mRNA, complete cds |
| R77293 | Intercellular adhesion molecule 1 (CD54), human rhinovirus receptor |
| AA436187 | Integrin, alpha M (complement component receptor 3, alpha; also known as CD11b |
| | (p170), macrophage antigen alpha polypeptide) |
| AA676470 | H. sapiens IAI.3B mRNA |
| AA443634 | Homo sapiens ubiquitin conjugating enzyme G2 (UBE2G2) mRNA, complete cds |
| AA664180 | Glutathione peroxidase 3 (plasma) |
| W58658 | H. sapiens mRNA for CLPP |
| H54023 | Homo sapiens monocyte/macrophage Ig-related receptor MIR-10 (MIR cl-10) mRNA, |
| | complete cds |
| H73724 | Cyclin-dependent kinase 6 |
| T70031 | Human neutral amino acid transporter B mRNA, complete cds |
| AA481758 | DNAJ PROTEIN HOMOLOG 1 |
| AA521431 | Human profilin mRNA, complete cds |
| AA446103 | ERGIC-53 PROTEIN PRECURSOR |
| N92646 | Immunoglobulin gamma 3 (Gm marker) |
| AA453789 | Protein-tyrosine kinase 7 |
| AA425299 | Homo sapiens ezrin-radixin-moesin binding phosphoprotein-50 mRNA, complete cds |
| AA868929 | Troponin T1, skeletal, slow |
| R60019 | Homolog 2 of Drosophila large discs |
| AA857343 | Human putative RNA binding protein (RBP56) mRNA, complete cds |
| AA481438 | Complement component 1 inhibitor (angioedema, hereditary) |
| AA399674 | Human small proline rich protein (sprII) mRNA, clone 1292 |
| T98887 | Glucose-6-phosphatase |
| AA676404 | Peptidylprolyl isomerase C (cyclophilin C) |
| H15747 | Human HU-K4 mRNA, complete cds |
| H16958 | Human glyceraldehyde 3-phosphate dehydrogenase mRNA |
| AA936783 | Eukaryotic translation initiation factor 3 (eIF-3) p36 subunit |
| AA884709 | Cytochrome P450 11 beta |
| H24688 | Human SWI/SNF complex 170 KDa subunit (BAF170) mRNA, complete cds |
| AA884403 | Human cardiotrophin-1 (CTF1) mRNA, complete cds |
| AA404619 | 5′ nucleotidase (CD73) |
| AA598611 | IMMEDIATE-EARLY RESPONSE PROTEIN NOT |
| H72875 | GATA-binding protein 3 |
| H63361 | Eukaryotic translation initiation factor 2B (eIF-2B) alpha subunit |
| R39221 | Human MAP kinase mRNA, complete cds |
| R02346 | U1 snRNP 70K protein |
| R51835 | unknown EST |
| R33031 | H. sapiens mRNA for sigma 3B protein |
| AA412053 | CD9 antigen |
| AA001897 | Erythroid alpha-spectrin |
| W81191 | Homo sapiens nucleolar autoantigen No55 mRNA, complete cds |
| AA430552 | Homo sapiens proline-rich Gla protein 2 (PRGP2) mRNA, complete cds |
| AA394130 | Human transducin-like protein mRNA, complete cds |
| N92864 | Human cleavage and polyadenylation specificity factor mRNA, complete cds |
| AA457123 | VALYL-TRNA SYNTHETASE |
| R43320 | Human guanine nucleotide-binding regulatory protein (Go-alpha) gene |
| AA670430 | Glutamate receptor, metabotropic 3 |
| H65066 | Visinin-like 1 |
| AA458785 | GUANYLATE CYCLASE SOLUBLE, BETA-1 CHAIN |
| AA485871 | H. sapiens mRNA for myosin-I beta |
| T39411 | Human 53K isoform of Type II phosphatidylinositol-4-phosphate 5-kinase (PIPK) |
| | mRNA, complete cds |
| R00855 | Homo sapiens 59 protein mRNA, 3′ end |
| H98666 | Metallopeptidase 1 (33 kD) |
| H72028 | GELSOLIN PRECURSOR, PLASMA |
| AA679177 | Human follistatin-related protein precursor mRNA, complete cds |
| N21576 | Human mitochondrial 1,25-dihydroxyvitamin D3 24-hydroxylase mRNA, complete cds |
| AA007419 | Human RGP4 mRNA, complete cds |
| T49657 | Homo sapiens TWIK-related acid-sensitive K+ channel (TASK) mRNA, complete cds |
| N38959 | Homo sapiens chaperonin containing t-complex polypeptide 1, beta subunit (Cctb) |
| | mRNA, complete cds |
| R51912 | Human somatostatin I gene and flanks |
| H90415 | Breast cancer 1, early onset |
| H41489 | Adaptin, beta 1 (beta prime) |
| H15456 | CALPAIN 1, LARGE |
| W45415 | ELASTASE IIIB PRECURSOR |
| AA447751 | Tyrosine hydroxylase |
| AA487486 | Cyclin D1 (PRAD1; parathyroid adenomatosis 1) |
| R56604 | Cholinergic receptor, nicotinic, alpha polypeptide 4 |
| T65772 | pulmonary surfactant protein (SP5) |
| H15085 | ADP-ribosylation factor 4-like |
| R61295 | Human ADP/ATP translocase mRNA, 3′ end, clone pHAT8 |
| T61256 | H. sapiens KHK mRNA for ketohexokinase, clone pHKHK3a |
| AA405731 | Phosphoenolpyruvate carboxykinase 1 (soluble) |
| T71879 | Complement component C2 |
| R59927 | Human mRNA for cytochrome c oxidase subunit VIc |
| AA496780 | Human small GTP binding protein Rab7 mRNA, complete cds |
| AA176688 | Human mRNA for lysosomal sialoglycoprotein, complete cds |
| AA436163 | Homo sapiens Pig12 (PIG12) mRNA, complete cds |
| AA428778 | Human placenta LERK-2 (EPLG2) mRNA, complete cds |
| AA463225 | Bone morphogenetic protein 4 |
| AA485426 | Interferon (alpha, beta and omega) receptor 2 |
| W47485 | Human sigma receptor mRNA, complete cds |
| H84982 | Human checkpoint suppressor 1 mRNA, complete cds |
| AA504615 | Homo sapiens mRNA for CAB1, complete cds |
| H94487 | Cathepsin E |
| AA448959 | Homo sapiens NADH: ubiquinone oxidoreductase 15 kDa IP subunit mRNA, nuclear |
| | gene encoding mitochondrial protein, complete cds |
| AA070358 | Transketolase (Wernicke-Korsakoff syndrome) |
| AA453401 | Human PH-20 homolog (LUCA2) mRNA, partial cds |
| N66737 | Collagen, type II, alpha 1 (primary osteoarthritis, spondyloepiphyseal dysplasia, |
| | congenital) |
| AA666180 | Human v-erbA related ear-2 gene |
| AA857131 | Human Tat-SF1 mRNA, complete cds |
| AA479102 | Protein kinase C, beta 1 |
| AA456077 | Homo sapiens mRNA for p27, complete cds |
| R87497 | H. sapiens mRNA for 2.19 gene |
| AA718910 | Human tax1-binding protein TXBP181 mRNA, complete cds |
| AA406269 | Nuclear factor I/X (CCAAT-binding transcription factor) |
| N74623 | Insulin-like growth factor 2 (somatomedin A) |
| H99364 | Human chloride channel protein (CLCN7) mRNA, partial cds |
| AA447684 | Small proline-rich protein 1B (cornifin) |
| AA282301 | Homo sapiens nuclear dual-specificity phosphatase (SBF1) mRNA, partial cds |
| H99588 | Human lymphoid nuclear protein (LAF-4) mRNA, complete cds |
| N53512 | Homo sapiens alpha 2 delta calcium channel subunit isoform I mRNA, complete cds |
| AA683321 | Homo sapiens PAR-5 mRNA, probable 5′ end |
| AA608557 | Damage-specific DNA binding protein 1 (127 kD) |
| AA757764 | Homo sapiens mRNA for DNA-binding protein, complete cds |
| AA406064 | Homo sapiens testis-specific Basic Protein Y 1 (BPY1) mRNA, complete cds |
| N54596 | Human Krueppel-related zinc finger protein (H-plk) mRNA, complete cds |
| AA481988 | Transcription factor 7 (T-cell specific) |
| N62394 | Gap junction protein, beta 1, 32 kD (connexin 32, Charcot-Marie-Tooth neuropathy, X- |
| | linked) |
| N26148 | Zinc finger protein 148 (pHZ-52) |
| AA496678 | B-cell CLL/lymphoma 3 |
| AA400973 | NEUTROPHIL GELATINASE-ASSOCIATED LIPOCALIN PRECURSOR |
| AA497027 | Human mRNA, clone HH109 (screened by the monoclonal antibody of insulin |
| | receptor substrate-1 (IRS-1)) |
| N64508 | Homo sapiens podocalyxin-like protein mRNA, complete cds |
| AA033564 | H. sapiens mRNA for DGCR6 protein |
| AA446108 | Endoglin (Osler-Rendu-Weber syndrome 1) |
| AA159577 | Mucin 5, subtype B, tracheobronchial |
| R36958 | unknown EST |
| AA629808 | Ribosomal protein L6 |
| AA482067 | Human tazarotene-induced gene 2 (TIG2) mRNA, complete cds |
| AA669314 | ATP synthase, H+ transporting, mitochondrial F1 complex, delta subunit |
| AA775241 | Aldolase A |
| R73584 | Homo sapiens hydroxysteroid sulfotransferase SULT2B1a (HSST2) mRNA, complete |
| | cds |
| H28984 | PHOSPHATIDYLSERINE SYNTHASE I |
| R44202 | Homo sapiens catechol-O-methyltransferase (COMT) mRNA, complete cds |
| W70051 | H. sapiens mRNA for M-phase phosphoprotein, mpp9 |
| AA401972 | Human RalGDS-like 2 (RGL2) mRNA, partial cds |
| AA236164 | CATHEPSIN S PRECURSOR |
| R22412 | Platelet/endothelial cell adhesion molecule (CD31 antigen) |
| AA424804 | MULTIDRUG RESISTANCE-ASSOCIATED PROTEIN 1 |
| AA669443 | Eukaryotic translation initiation factor 5 (elF5) |
| N69689 | RAS-RELATED PROTEIN RAB-1A |
| H24316 | AQUAPORIN-CHIP |
| AA074224 | Recoverin |
| R36571 | Human U1 snRNP-specific protein A gene |
| AA056465 | Human 54 kDa protein mRNA, complete cds |
| AA633811 | H. sapiens E4BP4 gene |
| AA457155 | Human zinc-finger protein C2H2-150 mRNA, complete cds |
| AA459104 | 60S RIBOSOMAL PROTEIN L13 |
| R40212 | Human coatomer protein (HEPCOP) mRNA, complete cds |
| AA086476 | Adenosine monophosphate deaminase 1 (isoform M) |
| AA663310 | Thymidylate synthase |
| AA455640 | Homo sapiens signalosome subunit 3 (Sgn3) mRNA, complete cds |
| AA496879 | Human (clone E5.1) RNA-binding protein mRNA, complete cds |
| AA085749 | Homo sapiens mRNA for ATP binding protein, complete cds |
| AA425755 | Homo sapiens mRNA for leukemia associated gene 1 |
| N52350 | H. sapiens mRNA for protein-tyrosine-phosphatase (tissue type: testis) |
| AA630104 | Lipase A, lysosomal acid, cholesterol esterase (Wolman disease) |
| AA454854 | ALPHA-AMYLASE 2B PRECURSOR |
| W73406 | DIHYDROPRYRIDINE-SENSITIVE L-TYPE, SKELETAL MUSCLE CALCIUM |
| | CHANNEL GAMMA SUBUNIT |
| R12802 | Human cytochrome bc-1 complex core protein II mRNA, complete cds |
| AA465355 | Homo sapiens mRNA for U3 snoRNP associated 55 kDa protein |
| AA829383 | DUAL SPECIFICITY MITOGEN-ACTIVATED PROTEIN KINASE KINASE 3 |
| AA629189 | Keratin 4 |
| AA430512 | Homo sapiens cytoplasmic antiproteinase 3 (CAP3) mRNA, complete cds |
| AA456439 | Human homozygous deletion target in pancreatic carcinoma (DPC4) mRNA, |
| | complete cds |
| H27864 | SECRETOGRANIN II PRECURSOR |
| AA644657 | MHC class I protein HLA-A (HLA-A28, -B40, -Cw3) |
| R40460 | Homo sapiens phosphatidylinositol 4-kinase mRNA, complete cds |
| W96058 | Human hnRNP H mRNA, complete cds |
| T72202 | Human transcription factor IL-4 Stat mRNA, complete cds |
| AA598794 | Connective tissue growth factor |
| AA599178 | Ribosomal protein L27a |
| R88247 | Adrenergic, beta, receptor kinase 1 |
| T98612 | Alpha-1 type 3 collagen |
| AA454856 | Phospholipid hydroperoxide glutathione peroxidase |
| N67048 | Type 3 iodothyronine deiodinase |
| AA778675 | Homo sapiens mRNA for calmegin, complete cds |
| H51117 | Human calmodulin dependent phosphodiesterase PDE1B1 mRNA, complete cds |
| N36174 | 5-HYDROXYTRYPTAMINE 2B RECEPTOR |
| AA777187 | Homo sapiens Cyr61 mRNA, complete cds |
| R09561 | Decay accelerating factor for complement (CD55, Cromer blood group system) |
| R16849 | Human HsPex13p mRNA, complete cds |
| AA884167 | ANNEXIN XIII |
| AA136983 | Cadherin 11 (OB-cadherin) |
| AA488622 | Human signal transducing adaptor molecule STAM mRNA, complete cds |
| AA699427 | Fructose-bisphosphatase 1 |
| AA490459 | Transcobalamin II |
| AA626787 | Human ras-related C3 botulinum toxin substrate (rac) mRNA, complete cds |
| N62179 | Human methylmalonate semialdehyde dehydrogenase gene, complete cds |
| N27190 | UBIQUITIN CARBOXYL-TERMINAL HYDROLASE ISOZYME L3 |
| AA441895 | Human glutathione-S-transferase homolog mRNA, complete cds |
| AA463924 | FACTOR VIII INTRON 22 PROTEIN |
| N78843 | Homo sapiens cyclophilin-33A (CYP-33) mRNA, complete cds |
| AA629719 | Cytochrome c oxidase VIIc subunit |
| AA464755 | Ankyrin 1, erythrocytic |
| AA459351 | H. sapiens sds22-like mRNA |
| AA488346 | MYOSIN LIGHT CHAIN ALKALI, SMOOTH-MUSCLE ISOFORM |
| AA427899 | Human mRNA fragment encoding beta-tubulin. (from clone D-beta-1) |
| AA453813 | H. sapiens mRNA for Gal-beta(1-3/1-4)GlcNAc alpha-2.3-sialyltransferase |
| AA397824 | Dopachrome tautomerase (dopachrome delta-isomerase, tyrosine-related protein 2) |
| AA633901 | Transforming growth factor, beta-induced, 68 kD |
| AA181334 | Troponin I (skeletal fast) |
| AA292410 | Clusterin (complement lysis inhibitor; testosterone-repressed prostate message 2; |
| | apolipoprotein J) |
| AA253434 | HEAT SHOCK FACTOR PROTEIN 2 |
| AA455056 | H. sapiens mRNA for MAP kinase activated protein kinase |
| R55188 | Human pre-T/NK cell associated protein (3B3) mRNA, 3′ end |
| AA465723 | Homo sapiens mRNA for protein phosphatase 2C gamma |
| N49856 | SODIUM-AND CHLORIDE-DEPENDENT BETAINE TRANSPORTER |
| AA455272 | H. sapiens mRNA for ITBA1 protein |
| AA459292 | CDC28 protein kinase 1 |
| AA878561 | Ubiquitin A-52 residue ribosomal protein fusion product 1 |
| AA772066 | Human phosphatidylinositol (4,5)bisphosphate 5-phosphatase homolog mRNA, |
| | partial cds |
| N78621 | H. sapiens mRNA for gamma-adaptin |
| AA291490 | H. sapiens mRNA for processing a-glucosidase I |
| N46828 | Homo sapiens mRNA for inositol 1,4,5-trisphosphate 3-kinase isoenzyme, partial cds |
| AA150487 | Alkaline phosphatase, placental (Regan isozyme) |
| AA282537 | MYOCYTE-SPECIFIC ENHANCER FACTOR 2 |
| AA707922 | Human mRNA for cone-specific cGMP phosphodiesterase gamma subunit, complete |
| | cds |
| AA443638 | Homo sapiens breast cancer-specific protein 1 (BCSG1) mRNA, complete cds |
| W73892 | Human putative tumor suppressor (LUCA15) mRNA, complete cds |
| N70734 | Troponin T2 (cardiac) |
| H57136 | Human phospholemman chloride channel mRNA, complete cds |
| AA709414 | Nidogen (enactin) |
| W65461 | Human protein tyrosine phosphatase mRNA, complete cds |
| AA436564 | Human cellular proto-oncogene (c-mer) mRNA, complete cds |
| AA029042 | Human hSIAH2 mRNA, complete cds |
| AA427725 | Homo sapiens carboxypeptidase Z precursor, mRNA, complete cds |
| N51280 | ADP-ribosylation factor like 1 |
| AA281347 | H. sapiens mRNA for MHC class I promoter binding protein |
| AA402960 | Human HLA class III region containing NOTCH4 gene, partial sequence, homeobox |
| | PBX2 (HPBX) gene, receptor for advanced glycosylation end products (RAGE) gene, |
| | complete cds, and 6 unidentified cds |
| N98485 | Human forkhead protein FREAC-2 mRNA, partial cds |
| AA490209 | H. sapiens mRNA for Sop2p-like protein |
| W61361 | Homo sapiens cytoplasmic antiproteinase 2 (CAP2) mRNA, complete cds |
| N51018 | Biglycan |
| AA455281 | DEFENDER AGAINST CELL DEATH 1 |
| W69471 | V-ski avian sarcoma viral oncogene homolog |
| AA486321 | Vimentin |
| AA458982 | Solute carrier family 9 (sodium/hydrogen exchanger), isoform 1 (antiporter, Na+/H+, |
| | amiloride sensitive) |
| AA442095 | NEDD-4 PROTEIN |
| N99003 | Active BCR-related gene |
| AA609284 | Homo sapiens mRNA for Eph-family protein, complete cds |
| AA195036 | Human Ro/SSA ribonucleoprotein homolog (RoRet) mRNA, complete cds |
| AA478268 | Human CtBP mRNA, complete cds |
| AA608583 | Homo sapiens mRNA for OTK27, complete cds |
| AA486435 | Homo sapiens mRNA for CDEP, complete cds |
| AA505045 | Human L2-9 transcript of unrearranged immunoglobulin V(H)5 pseudogene |
| AA487893 | TUMOR-ASSOCIATED ANTIGEN L6 |
| AA292226 | Homo sapiens creatine transporter mRNA, complete cds |
| H87106 | Homo sapiens T245 protein (T245) mRNA, complete cds |
| W96450 | Human putative tRNA synthetase-like protein mRNA, complete cds |
| N33331 | Human peroxisome proliferator activated receptor mRNA, complete cds |
| AA405800 | Dodecenoyl-Coenzyme A delta isomerase (3,2 trans-enoyl-Coenzyme A isomerase) |
| T51539 | Macrophage stimulating 1 (hepatocyte growth factor-like) |
| N59764 | Human guanosine 5′-monophosphate synthase mRNA, complete cds |
| AA521346 | H. sapiens mRNA for Ndr protein kinase |
| AA428551 | Homo sapiens SOX22 protein (SOX22) mRNA, complete cds |
| AA489383 | Bone morphogenetic protein 2 |
| AA490172 | Collagen, type I, alpha-2 |
| AA504477 | Human cytoskeleton associated protein (CG22) mRNA, complete cds |

List of Table III related sequences:

| Accession_{—} | | |
|---|---|---|
| mrgd | NAME | Gene |
| M98539 | Human prostaglandin D2 synthase gene, exon 7 | |
| AB004922 | Homo sapiens gene for Smad 3, exon 1, partial sequence | |
| AB006000 | Homo sapiens mRNA for chondromodulin-I precursor, complete cds | |
| AB017364 | Homo sapiens mRNA for frizzled-2, complete cds | |
| AB020236 | Homo sapiens gene for ribosomal protein L27A, complete cds | Ribosomal protein L27a |
| AB042820 | Homo sapiens RPL6 gene for ribosomal protein L6, complete cds | Ribosomal protein L6 |
| AB043547 | Homo sapiens gene for SMAD4, partial cds | |
| AB080265 | Homo sapiens CYP2J2 mRNA for cytochrome P450 2J2, complete cds | Cytochrome P450, subfamily IIJ |
| | | (arachidonic acid epoxygenase) polypeptide 2 |
| AF000979 | Homo sapiens testis-specific Basic Protein Y 1 (BPY1) mRNA, complete cds | Homo sapiens testis-specific Basic Protein |
| | | Y 1 (BPY1) mRNA, complete cds |
| AF001450 | Homo sapiens core binding factor alpha1 | |
| | subunit (CBFA1) gene, exon 7 and complete cds | |
| AF004231 | Homo sapiens monocyte/macrophage Ig-related receptor MIR-10 (MIR cl-10) | Homo sapiens monocyte/macrophage Ig-related |
| | mRNA, complete cds | receptor MIR-10 (MIR cl-10) mRNA, |
| | | complete cds |
| AF009801 | Homo sapiens homeodomain protein (BAPX1) mRNA, complete cds | |
| AF010126 | Homo sapiens breast cancer-specific protein 1 (BCSG1) mRNA, complete cds | Homo sapiens breast cancer-specific |
| | | protein 1 (BCSG1) mRNA, complete cds |
| AF010316 | Homo sapiens Pig12 (PIG12) mRNA, complete cds | Homo sapiens Pig12 (PIG12) mRNA, |
| | | complete cds |
| AF013591 | Homo sapiens homolog of the Aspergillus nidulans sudD gene product mRNA, | Homo sapiens homolog of the |
| | complete cds | Aspergillus nidulans sudD gene product |
| | | mRNA, complete cds |
| AF037204 | Homo sapiens RING zinc finger protein (RZF) mRNA, complete cds | |
| AF043339 | Homo sapiens macrophage | |
| | inflammatory protein 1 alpha (MIP1a) mRNA, partial cds | |
| AF049656 | Homo sapiens inducible nitric oxide synthase (iNOS) mRNA, complete cds | |
| AF072872 | Homo sapiens frizzled 1 mRNA, complete cds | |
| AF188285 | Homo sapiens bone morphogenetic protein 9 (BMP9) mRNA, complete cds | |
| AF189279 | Homo sapiens group IIE secretory phospholipase A2 mRNA, complete cds | |
| AF248634 | Homo sapiens syndecan 3 (SDC3) mRNA, complete cds | |
| AF304431 | Homo sapiens hypoxia-inducible factor | |
| | 1 alpha subunit (HIF1A) mRNA, complete cds | |
| AF339054 | Homo sapiens BCL2-associated X protein (BAX) gene, exons 1, 2 and partial cds | |
| AF348700 | Homo sapiens ubiquitin A-52 residue ribosomal protein fusion product 1 | Ubiquitin A-52 residue ribosomal protein |
| | (UBA52), mRNA, complete cds | fusion product 1 |
| AF395008 | Homo sapiens interleukin 4 (IL4) gene, complete cds | |
| AF405705 | Homo sapiens matrix metalloproteinase 3 | |
| | (stromelysin 1, progelatinase) (MMP3) gene, complete cds | |
| AF411526 | Homo sapiens nerve growth factor beta (NGFB) mRNA, complete cds | Nerve growth factor beta |
| AF469046 | Homo sapiens macrophage migration inhibitory factor (MIF) mRNA, complete cds | |
| AF477981 | Homo sapiens osterix mRNA, complete cds | |
| AJ279016 | Homo sapiens mRNA for chondrocyte expressed protein 68 kDa (CEP-68 gene) | |
| AY043326 | Homo sapiens keratin 4 (KRT4) gene, complete cds | Keratin 4 |
| AY044847 | Homo sapiens aggrecanase 1 (ADAMTS4) gene, complete cds | |
| D13748 | Human mRNA for eukaryotic initiation factor 4AI | Human mRNA for eukaryotic |
| | | initiation factor 4AI |
| D38255 | Homo sapiens mRNA for CAB1, complete cds | Homo sapiens mRNA for CAB1, complete cds |
| D45399 | Human mRNA for cone-specific cGMP phosphodiesterase gamma subunit, | Human mRNA for cone-specific cGMP |
| | complete cds | phosphodiesterase gamma subunit, |
| | | complete cds |
| D49738 | Human cytoskeleton associated protein (CG22) mRNA, complete cds | Human cytoskeleton associated protein |
| | | (CG22) mRNA, complete cds |
| D49835 | Homo sapiens mRNA for DNA-binding protein, complete cds | Homo sapiens mRNA for DNA-binding |
| | | protein, complete cds |
| D90040 | Human mRNA for arylamine N-acetyltransferase (EC 2.3.1.5) | ARYLAMINE N-ACETYLTRANSFERASE, |
| | | MONOMORPHIC |
| J00306 | Human somatostatin I gene and flanks | Human somatostatin I gene and flanks |
| J03191 | Human profilin mRNA, complete cds | Human profilin mRNA, complete cds |
| J03592 | Human ADP/ATP translocase mRNA, 3′ end, clone pHAT8 | Human ADP/ATP translocase mRNA, |
| | | 3′ end, clone pHAT8 |
| J04111 | Human c-jun proto oncogene (JUN), complete cds, clone hCJ-1 | |
| J04177 | Human alpha-1 type XI collagen (COL11A1) mRNA, complete cds | |
| J04973 | Human cytochrome bc-1 complex core protein II mRNA, complete cds | Human cytochrome bc-1 complex core |
| | | protein II mRNA, complete cds |
| J05036 | Human cathepsin E mRNA, complete cds | Cathepsin E |
| K00065 | Human superoxide dismutase (SOD-1) mRNA, complete cds | Human superoxide dismutase (SOD-1) mRNA, |
| | | complete cds |
| K00650 | Human fos proto-oncogene (c-fos), complete cds | |
| L05095 | Homo sapiens ribosomal protein L30 mRNA, complete cds | 60S RIBOSOMAL PROTEIN L30 |
| L08895 | Homo sapiens MADS/MEF2-family | |
| | transcription factor (MEF2C) mRNA, complete cds | |
| L10347 | Human pro-alpha1 type II collagen (COL2A1) gene exons 1-54, complete cds | |
| L11566 | Homo sapiens ribosomal protein L18 (RPL18) mRNA, complete cds | 60S RIBOSOMAL PROTEIN L18 |
| L13286 | Human mitochondrial 1,25-dihydroxyvitamin D3 24-hydroxylase mRNA, | Human mitochondrial 1,25-dihydroxyvitamin |
| | complete cds | D3 24-hydroxylase mRNA, complete cds |
| L13463 | Human helix-loop-helix basic phosphoprotein (G0S8) mRNA, complete cds | |
| L13616 | Human focal adhesion kinase (FAK) mRNA, complete cds | |
| L13720 | Homo sapiens growth-arrest-specific protein (gas) mRNA, complete cds | Homo sapiens growth-arrest-specific protein |
| | | (gas) mRNA, complete cds |
| L22009 | Human hnRNP H mRNA, complete cds | Human hnRNP H mRNA, complete cds |
| L28997 | Homo sapiens ARL1 mRNA, complete cds | ADP-ribosylation factor like 1 |
| L31409 | Homo sapiens creatine transporter mRNA, complete cds | Homo sapiens creatine transporter mRNA, |
| | | complete cds |
| L33930 | Homo sapiens CD24 signal transducer mRNA, complete cds and 3′ region | |
| L34059 | Homo sapiens cadherin-4 mRNA, complete cds | |
| L41162 | Homo sapiens collagen alpha 3 type IX (COL9A3) mRNA, complete cds | |
| L47647 | Homo sapiens creatine kinase B mRNA, complete cds | Creatine kinase B |
| M13994 | Human B-cell leukemia/lymphoma 2 (bcl-2) proto-oncogene | |
| | mRNA encoding bcl-2-alpha protein, complete cds | |
| M14144 | Human vimentin gene, complete cds | Vimentin |
| M14631 | Human guanine nucleotide-binding protein G-s, alpha subunit mRNA, partial | Human guanine nucleotide-binding protein G-s, |
| | cds | alpha subunit mRNA, partial cds |
| M16652 | Human pancreatic elastase IIA mRNA, complete cds | Elastase 1, pancreatic (elastase IIA) |
| M20137 | Human interleukin 3 (IL-3) mRNA, complete cds, clone pcD-SR-alpha | |
| M22636 | Human U1 small nuclear ribonucleoprotein 70 kd protein mRNA, complete cds | U1 snRNP 70K protein |
| M37825 | Human fibroblast growth factor-5 (FGF-5) mRNA, complete cds | |
| M57293 | Human parathyroid hormone-related peptide | |
| | (PTHRP) gene, exons 1A, 1B, 1C, and 2 | |
| M58458 | Human ribosomal protein S4 (RPS4X) isoform mRNA, complete cds | Ribosomal protein S4, X-linked |
| M58525 | Homo sapiens catechol-O-methyltransferase (COMT) mRNA, complete cds | Homo sapiens catechol-O-methyltransferase |
| | | (COMT) mRNA, complete cds |
| M58549 | Human matrix Gla protein (MGP) mRNA, complete cds | |
| M61877 | Human erythroid alpha-spectrin (SPTA1) mRNA, complete cds | Erythroid alpha-spectrin |
| M62402 | Human insulin-like growth factor binding protein 6 (IGFBP6) mRNA, complete | Insulin-like growth factor binding protein 6 |
| | cds | |
| M65062 | Human insulin-like growth factor binding protein 5 (IGFBP-5) mRNA, complete cds | |
| M65217 | Human heat shock factor 2 (HSF2) mRNA, complete cds | HEAT SHOCK FACTOR PROTEIN 2 |
| M76701 | Homo sapiens zinc finger protein 35 (ZNF35) gene, exon 1 | |
| M77016 | Human tropomodulin mRNA, complete cds | Tropomodulin |
| M81768 | Human Na/H antiporter (APNH1) mRNA, complete cds | Human Na/H antiporter (APNH1) mRNA, |
| | | complete cds |
| M84489 | Human extracellular signal-regulated kinase 2 mRNA, complete cds | |
| M84721 | Human AMP deaminase (AMPD3) mRNA, complete cds | Adenosine monophosphate deaminase |
| | | (isoform E) |
| M87842 | Human S-lac lectin L-14-II (LGALS2) mRNA, complete cds | GALECTIN-2 |
| M92934 | Human connective tissue growth factor, complete cds | Connective tissue growth factor |
| M95610 | Human alpha 2 type IX collagen (COL9A2) mRNA, partial cds | |
| M96684 | H. sapiens Pur (pur-alpha) mRNA, complete cds | H. sapiens Pur (pur-alpha) mRNA, |
| | | complete cds |
| M97676 | Homo sapiens (region 7) homeobox protein (HOX7) mRNA, complete cds | |
| NM_000194 | Homo sapiens hypoxanthine phosphoribosyltransferase | |
| | 1 (Lesch-Nyhan syndrome) (HPRT1), mRNA | |
| NM_000213 | Homo sapiens integrin, beta 4 (ITGB4), mRNA | |
| NM_000221 | Homo sapiens ketohexokinase (fructokinase) (KHK), transcript variant a, | H. sapiens KHK mRNA for ketohexokinase, |
| | mRNA | clone pHKHK3a |
| NM_000235 | Homo sapiens lipase A, lysosomal acid, cholesterol esterase (Wolman | Lipase A, lysosomal acid, cholesterol esterase |
| | disease) (LIPA), mRNA | (Wolman disease) |
| NM_000358 | Homo sapiens transforming growth factor, beta-induced, 68 kD (TGFBI), mRNA | Transforming growth factor, |
| | | beta-induced, 68 kD |
| NM_000364 | Homo sapiens troponin T2, cardiac (TNNT2), mRNA | Troponin T2 (cardiac) |
| NM_000537 | Homo sapiens renin (REN), mRNA | RENIN PRECURSOR, RENAL |
| NM_000574 | Homo sapiens decay accelerating factor for complement (CD55, Cromer blood | Decay accelerating factor for complement |
| | group system) (DAF), mRNA | (CD55, Cromer blood group system) |
| NM_000600 | Homo sapiens interleukin 6 (interferon, beta 2) (IL6), mRNA | |
| NM_000618 | Homo sapiens insulin-like growth factor 1 (somatomedia C) (IGF1), mRNA | |
| NM_000632 | Homo sapiens Integrin, alpha M (complement component receptor 3, alpha; | Integrin, alpha M (complement component |
| | also known as CD11b (p170), macrophage antigen alpha polypeptide) | receptor 3, alpha; also known as CD11b |
| | (ITGAM), mRNA | (p170), polypeptide) macrophage antigen alpha |
| NM_000711 | Homo sapiens bone gamma-carboxyglutamate | |
| | (gla) protein (osteocalcin) (BGLAP), mRNA | |
| NM_000962 | Homo sapiens prostaglandin-endoperoxide synthase 1 (prostaglandin G/H | Prostaglandin-endoperoxide synthase 1 |
| | synthase and cyclooxygenase) (PTGS1), transcript variant 1, mRNA | (prostaglandin G/H synthase and |
| | | cyclooxygenase) |
| MM_000977 | Homo sapiens ribosomal protein L13 (RPL13), transcript variant 1, mRNA | 60S RIBOSOMAL PROTEIN L13 |
| NM_000996 | Homo sapiens ribosomal protein L35a (RPL35A), mRNA | Ribosomal protein L35a |
| NM_001012 | Homo sapiens ribosomal protein S8 (RPS8), mRNA | 40S RIBOSOMAL PROTEIN S8 |
| NM_001025 | Homo sapiens ribosomal protein S23 (RPS23), mRNA | 40S RIBOSOMAL PROTEIN S23 |
| NM_001064 | Homo sapiens transketolase (Wernicke-Korsakoff syndrome) (TKT), mRNA | Transketolase (Wernicke-Korsakoff syndrome) |
| NM_001127 | Homo sapiens adaptor-related protein complex 1, beta 1 subunit | Adaptin, beta 1 (beta prime) |
| | | AP1B1), mRNA |
| NM_001200 | Homo sapiens bone morphogenetic protein 2 (BMP2), mRNA | Bone morphogenetic protein 2 |
| NM_001229 | Homo sapiens caspase 9, apoptosis-related cysteine protease | |
| | (CASP9), transcript variant alpha, mRNA | |
| NM_001511 | Homo sapiens GRO1 oncogene (melanoma growth | |
| | stimulating activity, alpha) (GRO1), mRNA | |
| NM_001565 | Homo sapiens small inducible cytokine subfamily B (Cys-X-Cys), member 10 | Interferon (gamma)-induced cell line; |
| | (SCYB10), mRNA | protein 10 from |
| NM_001632 | Homo sapiens alkaline phosphatase, placental (Regan isozyme) (ALPP), | Alkaline phosphatase, placental |
| | mRNA | (Regan isozyme) |
| NM_001687 | Homo sapiens ATP synthase, H+ transporting, mitochondrial F1 complex, delta | ATP synthase, H+ transporting, |
| | subunit (ATP5D), mRNA | mitochondrial F1 complex, delta subunit |
| NM_001718 | Homo sapiens bone morphogenetic protein 6 (BMP6), mRNA | |
| NM_001745 | Homo sapiens calcium modulating ligand (CAMLG), mRNA | Calcium modulating ligand |
| NM_001797 | Homo sapiens cadherin 11, type 2, OB-cadherin | |
| | (osteoblast) (CDH11), transcript variant 1, mRNA | |
| NM_001844 | Homo sapiens collagen, type II, alpha 1 (primary osteoarthritis, | |
| | spondyloepiphyseal dysplasia, congenital) (COL2A1), transcript variant 1, mRNA | |
| NM_001912 | Homo sapiens cathepsin L (CTSL), mRNA | |
| NM_001969 | Homo sapiens eukaryotic translation initiation factor 5 (EIF5), mRNA | Eukaryotic translation initiation factor 5 (eIF5) |
| NM_002073 | Homo sapiens guanine nucleotide binding protein (G protein), alpha z | Guanine nucleotide binding protein |
| | polypeptide (GNAZ), mRNA | polypeptide (G protein), alpha z |
| NM_002094 | Homo sapiens G1 to S phase transition 1 (GSPT1), mRNA | G1 to S phase transition 1 |
| NM_002160 | Homo sapiens hexabrachion (tenascin C, cytotactin) (HXB), mRNA | |
| NM_002211 | Homo sapiens integrin, beta 1 (fibronectin receptor, beta polypeptide, | |
| | antigen CD29 includes MDF2, MSK12) (ITGB1), mRNA | |
| NM_002379 | Homo sapiens matrilin 1, cartilage matrix protein (MATN1), mRNA | |
| NM_002381 | Homo sapiens matrilin 3 (MATN3) precursor, mRNA | |
| NM_002421 | Homo sapiens matrix metalloproteinase 1 (interstitial collagenase) (MMP1), mRNA | |
| NM_002424 | Homo sapiens matrix metalloproteinase 8 (neutrophil collagenase) (MMP8), mRNA | |
| NM_002427 | Home sapiens matrix metalloproteinase 13 (collagenase 3) (MMP13), mRNA | |
| NM_002591 | Homo sapiens phosphoenolpyruvate carboxykinase 1 (soluble) (PCK1), mRNA | Phosphoenolpyruvate carboxykinase 1 (soluble) |
| | mRNA carboxykinase 1 (soluble) | |
| NM_002619 | Home sapiens platelet factor 4 (PF4), mRNA | Platelet factor 4 |
| NM_002722 | Homo sapiens pancreatic polypeptide (PPY), mRNA | |
| NM_002738 | Homo sapiens protein kinase C, beta 1 (PRKCB1), mRNA | Protein kinase C, beta 1 |
| NM_002903 | Homo sapiens recoverin (RCV1), mRNA | Recoverin |
| NM_003036 | Homo sapiens v-ski sarcoma viral oncogene homolog (avian) (SKI), mRNA | V-ski avian sarcoma viral oncogene homolog |
| NM_003282 | Homo sapiens troponin I, skeletal, fast (TNNI2), mRNA | Troponin I (skeletal fast) |
| NM_003385 | Homo sapiens visinin-like 1 (VSNL1), | Visinin-like 1 |
| NM_003395 | Homo sapiens wingless-type MMTV integration | |
| | site family, member 14 (WNT14), mRNA | |
| NM_004613 | Homo sapiens transglutaminase 2 (C polypeptide, | |
| | protein-glutamine-gamma-glutamyltransferase) (TGM2), mRNA | |
| NM_004832 | Homo sapiens glutathione-S-transferase like; glutathione transferase omega | Human glutathione-S-transferase homolog |
| | (GSTTLp28), mRNA | cds mRNA, complete |
| NM_004994 | Homo sapiens matrix metalloproteinase 9 (gelatinase B, 92 kD gelatinase, | |
| | 92 kD type IV collagenase) (MMP9), mRNA | |
| NM_004995 | Homo sapiens matrix metalloproteinase 14 (membrane-inserted) (MMP14), mRNA | |
| NM_005038 | Homo sapiens peptidylprolyl isomerase D (cyclophilin D) (PPID), mRNA | 40 KD PEPTIDYL-PROLYL CIS-TRANS |
| | | ISOMERASE |
| NM_005186 | Homo sapiens calpain 1, (mu/l) large subunit (CAPN1), mRNA | CALPAIN 1, LARGE |
| NM_005346 | Homo sapiens heat shock 70 kD protein 1B (HSPA1B), mRNA | HEAT SHOCK 70 KD PROTEIN 1 |
| NM_005438 | Homo sapiens FOS-like antigen 1 (FOSL1), mRNA | |
| NM_005506 | Homo sapiens CD36 antigen (collagen type I receptor, thrombospondin | |
| | receptor)-like 2 (lysosomal integral membrane protein II) (CD36L2), mRNA | |
| NM_006289 | Homo sapiens talin 1 (TLN1), mRNA | |
| NM_006988 | Homo sapiens a disintegrin-like and metalloprotease (reprolysin type) | |
| | with thrombospondin type 1 motif, 1 (ADAMTS1), mRNA | |
| NM_007306 | Homo sapiens breast cancer 1, early onset (BRCA1), transcript variant | Breast cancer 1, early onset |
| | BRCA1-exon4, mRNA | |
| NM_007352 | Homo sapiens elastase 3B, pancreatic (ELA3B), Mrna | ELASTASE IIIB PRECURSOR |
| NM_014000 | Homo sapiens vinculin (VCL), transcript variant meta-VCL, mRNA | |
| NM_014470 | Homo sapiens GTP-binding protein (RHO6), mRNA | |
| NM_018952 | Homo sapiens homeo box B6 (HOXB6), mRNA | |
| NM_021019 | Homo sapiens myosin, light polypeptide 6, alkali, smooth muscle and non- | MYOSIN LIGHT CHAIN ALKALI, |
| | muscle (MYL6), transcript variant 1, mRNA | SMOOTH-MUSCLE ISOFORM |
| NM_033150 | Homo sapiens collagen, type II, alpha 1 (primary osteoarthritis, | |
| | spondyloepiphyseal dysplasia, congenital) (COL2A1), transcript variant 2, mRNA | |
| NM_053056 | Homo sapiens cyclin D1 (PRAD1: parathyroid adenomatosis 1) (CCND1), | Cyclin D1 (PRAD1; parathyroid |
| | mRNA | adenomatosis 1) |
| NM_080682 | Homo sapiens vascular cell adhesion | |
| | molecule 1 (VCAM 1), transcript variant 2, mRNA | |
| S79854 | Homo sapiens type 3 iodothyronine deiodinase mRNA, complete cds | Type 3 iodothyronine deiodinase |
| S83308 | SOX5 = Sry-related HMG box gene {alternatively | |
| | spliced} [human, testis, mRNA, 1473 nt] | |
| U07424 | Human putative tRNA synthetase-like protein mRNA, complete cds | Human putative tRNA synthetase-like |
| | | protein mRNA, complete cds |
| U07620 | Human MAP kinase mRNA, complete cds | Human MAP kinase mRNA, complete cds |
| U08023 | Human cellular proto-oncogene (c-mer) mRNA, complete cds | Human cellular proto-oncogene (c-mer) |
| | | mRNA, complete cds |
| U09303 | Human T cell leukemia LERK-2 (EPLG2) mRNA, complete cds | |
| U09577 | Homo sapiens lysosomal hyaluronidase (LUCA2/HYAL2) mRNA, complete cds | Human PH-20 homolog (LUCA2) mRNA, |
| | | partial cds |
| U09825 | Human acid finger protein mRNA, complete cds | Acid finger protein ZNF173 |
| U13261 | Homo sapiens eIF-2-associated p67 homolog mRNA, complete cds | Human eIF-2-associated p67 homolog |
| | | mRNA, complete cds |
| U13660 | Human cartilage-derived morphogenetic protein 1 (CDMP-1) mRNA, complete cds | |
| U13991 | Human TATA-binding protein associated factor 30 kDa subunit (tafII30) mRNA, | Human TATA-binding protein associated |
| | complete cds | factor 30 kDa subunit (tafII30) mRNA, |
| | | complete cds |
| U14966 | Human ribosomal protein L5 mRNA, complete cds | Ribosomal protein L5 |
| U14971 | Human ribosomal protein S9 mRNA, complete cds | |
| U15085 | Human HLA-DMB mRNA, complete cds | Major histocompatibility complex, |
| | | class II, DM beta |
| U16031 | Human transcription factor IL-4 Stat mRNA, complete cds | Human transcription factor IL-4 Stat |
| | | mRNA, complete cds |
| U16261 | Human MDA-7 (mda-7) mRNA, complete cds | |
| U18299 | Human damage-specific DNA binding protein DDBa p127 subunit (DDB1) | Damage-specific DNA binding protein |
| | mRNA, complete cds | 1 (127 kD) |
| U20980 | Human chromatin assembly factor-I p60 subunit mRNA, complete cds | Human chromatin assembly factor-I |
| | | p60 subunit mRNA, complete cds |
| U22409 | Human parathyroid hormone/PTH-related peptide receptor | |
| | (PTH/PTHrP) gene, exon 14 and complete cds | |
| U23028 | Human eukaryotic initiation factor 2B-epsilon mRNA, partial cds | Human eukaryotic initiation factor |
| | | 2B-epsilon mRNA, partial cds |
| U23946 | Human putative tumor suppressor (LUCA15) mRNA, complete cds | Human putative tumor suppressor |
| | | (LUCA15) mRNA, complete cds |
| U24152 | Human p21-activated protein kinase (Pak1) gene, complete cds | Human protein kinase PAK1 |
| | | mRNA, complete cds |
| U25789 | Human ribosomal protein L21 mRNA, complete cds | Ribosomal protein L21 |
| U27699 | Human pephBGT-1 betaine-GABA transporter mRNA, complete cds | SODIUM-AND CHLORIDE-DEPENDENT |
| | | BETAINE TRANSPORTER |
| U31202 | Human noggin (NOGGIN) gene, complete cds, (NOG) | |
| U32169 | Human pro-a2 chain of collagen type XI (COL11A2) gene, complete cds | |
| U32907 | Human p37NB mRNA, complete cds | Human p37NB mRNA, complete cds |
| U33822 | Human tax1-binding protein TXBP181 mRNA, complete cds | Human tax1-binding protein TXBP181 |
| | | mRNA, complete cds |
| U37012 | Human cleavage and polyadenylation specificity factor mRNA, complete cds | Human cleavage and polyadenylation |
| | | specificity factor mRNA, complete cds |
| U38864 | Human zinc-finger protein C2H2-150 mRNA, complete cds | Human zinc-finger protein C2H2-150 |
| | | mRNA, complete cds |
| U40373 | Human cell surface glycoprotein CD44 mRNA, complete cds | |
| U41517 | Human channel-like integral membrane protein (AQP-1) mRNA, clone AQP-1- | AQUAPORIN-CHIP |
| | 1656, complete cds | |
| U43148 | Human patched homolog (PTC) mRNA, complete cds | |
| U43747 | Human frataxin (FRDA) mRNA, complete cds | Friedreich ataxia |
| U43842 | Homo sapiens bone morphogenetic protein-4 (hBMP-4) gene, complete cds | Bone morphogenetic protein 4 |
| U45975 | Human phosphatidylinositol (4,5)bisphosphate 5-phosphatase homolog mRNA, | Human phosphatidylinositol |
| | partial cds | (4,5)bisphosphate 5- phosphatase homolog |
| | | mRNA, partial cds |
| U53204 | Human plectin (PLEC1) mRNA, complete cds | Human plectin (PLEC1) mRNA, complete cds |
| U53347 | Human neutral amino acid transporter B mRNA, complete cds | Human neutral amino acid transporter B |
| | | mRNA, complete cds |
| U59289 | Human H-cadherin mRNA, complete cds | |
| U59423 | Human Smad1 mRNA, complete cds | |
| U63717 | Homo sapiens osteoclast stimulating factor mRNA, complete cds | Human osteoclast stimulating factor |
| | | mRNA, complete cds |
| U68723 | Human checkpoint suppressor 1 mRNA, complete cds | |
| U70312 | Homo sapiens integrin binding protein Del-1 (Del1) mRNA, complete cds | |
| U72245 | Human phospholemman chloride channel mRNA, complete cds | Human phospholemman chloride channel |
| | | mRNA, complete cds |
| U75283 | Human sigma receptor mRNA, complete cds | Human sigma receptor mRNA, complete cds |
| U76992 | Human Tat-SF1 mRNA, complete cds | Human Tat-SF1 mRNA, complete cds |
| U80998 | Human basic helix-loop-helix DNA binding protein (TWIST) gene, complete cds | |
| U83460 | Human high-affinity copper uptake protein (hCTR1) mRNA, complete cds | Human high-affinity copper uptake protein |
| | | (hCTR1) mRNA, complete cds |
| U90547 | Human Ro/SSA ribonucleoprotein homolog (RoRet) mRNA, complete cds | Human Ro/SSA ribonucleoprotein homolog |
| | | (RoRet) mRNA, complete cds |
| U92268 | Homo sapiens mitogen activated protein kinase p38-2 mRNA, complete cds | |
| U93181 | Homo sapiens nuclear dual-specificity phosphatase (SBF1) mRNA, partial cds | Homo sapiens nuclear dual-specificity |
| | | phosphatase (SBF1) mRNA, partial cds |
| X00129 | Human mRNA for retinol binding protein (RBP) | |
| X00588 | Human mRNA for precursor of epidermal growth factor receptor | |
| X02910 | Human gene for tumor necrosis factor (TNF-alpha) | |
| X03742 | Human gene for L apoferritin exons 1 and 2 | |
| X04412 | Human mRNA for plasma gelsolin | GELSOLIN PRECURSOR, PLASMA |
| X06614 | Human mRNA for receptor of retinoic acid | Human mRNA for receptor of retinoic acid |
| X12794 | Human v-erbA related ear-2 gene | Human v-erbA related ear-2 gene |
| X14420 | Human mRNA for pro-alpha-1 type 3 collagen | |
| X51801 | Human OP-1 mRNA for osteogenic protein | |
| X54412 | Human mRNA for alpha1(IX) collagen (long form) | |
| X55654 | Homo sapiens mitochondrial coxII mRNA for cytochrome C oxidase II subunit | |
| X55764 | Human mRNA for cytochrome P-450 (11 Beta) | Cytochrome P450 11 beta |
| X58399 | Human L2-9 transcript of unrearranged immunoglobulin V(H)5 pseudogene | Human L2-9 transcript of unrearranged |
| | | immunoglobulin V(H)5 pseudogene |
| X58957 | H. sapiens atk mRNA for agammaglobulinaemia tyrosine kinase | Agammaglobulinaemia protein-tyrosine |
| | | kinase atk |
| X60188 | Human ERK1 mRNA for protein serine/threonine kinase | |
| X60382 | H. sapiens COL10A1 gene for collagen (alpha-1 type X) | |
| X67337 | H. sapiens HPBRII-4 mRNA | H. sapiens HPBRII-4 mRNA |
| X70683 | H. sapiens mRNA for SOX-4 protein | |
| X71661 | H. sapiens ERGIC-53 mRNA | ERGIC-53 PROTEIN PRECURSOR |
| X74795 | H. sapiens P1-Cdc46 mRNA | CDC46 HOMOLOG |
| X76770 | H. sapiens PAP mRNA | H. sapiens PAP mRNA |
| X78712 | H. sapiens mRNA for glycerol kinase testis specific 2 | Glycerol kinase 2 (testis specific) |
| X87237 | H. sapiens mRNA for processing a-glucosidase I | H. sapiens mRNA for processing |
| | | a-glucosidase I |
| X87342 | H. sapiens mRNA for human giant larvae homolog | H. sapiens mRNA for human |
| | | giant larvae homolog |
| X92475 | H. sapiens mRNA for ITBA1 protein | H. sapiens mRNA for ITBA1 protein |
| X94216 | H. sapiens mRNA for VEGF-C protein | |
| XM_001306 | Homo sapiens solute carrier family 16 (monocarboxylic acid transporters), | Solute carrier family 16 (monocarboxylic |
| | member 1 (SLC16A1), mRNA | acid transporters), member 1 |
| XM_001316 | Homo sapiens adenosine monophosphate deaminase 1 (isoform M) (AMPD1), | Adenosine monophosphate |
| | mRNA | deaminase 1 (isoform M) |
| XM_001324 | Homo sapiens calponin 3, acidic (CNN3), mRNA | Calponin 3, acidic |
| XM_001782 | Homo sapiens fibromodulin (FMOD), mRNA | |
| XM_001826 | Homo sapiens alkaline phosphatase, liver/bone/kidney (ALPL), mRNA | Alkaline phosphatase, liver/bone/kidney |
| XM_002321 | Homo sapiens glypican 1 (GPC1), mRNA | Glypican 1 |
| XM_003059 | Homo sapiens peroxisome proliferative | |
| | activated receptor, gamma (PPARG), mRNA | |
| XM_003222 | Homo sapiens catenin (cadherin-associated | |
| | protein), beta 1 (88 kD) (CTNNB1), mRNA | |
| XM_003730 | Homo sapiens cytochrome c oxidase subunit VIIc (COX7C), mRNA | Cytochrome c oxidase VIIc subunit |
| XM_003752 | Homo sapiens interleukin 3 (colony-stimulating factor, multiple) (IL3), mRNA | |
| XM_003913 | Homo sapiens integrin, alpha 2 (CD49B, alpha | |
| | 2 subunit of VLA-2 receptor) (ITGA2), mRNA | |
| XM_004063 | Homo sapiens early growth response 1 (EGR1), mRNA | |
| XM_006121 | Homo sapiens cathepsin D (lysosomal aspartyl protease) (CTSD), mRNA | |
| XM_009336 | Homo sapiens cartilage oligomeric matrix protein (pseudoachondroplasia, | |
| | epiphyseal dysplasia 1, multiple) (COMP), mRNA | |
| XM_009915 | Homo sapiens leukemia inhibitory factor | |
| | (cholinergic differentiation factor) (LIF), mRNA | |
| XM_010702 | Homo sapiens cathepsin K (pycnodysostosis) (CTSK), mRNA | |
| XM_012503 | Homo sapiens matrix metalloproteinase 2 (gelatinase A, 72 kD | |
| | gelatinase, 72 kD type IV collagenase) (MMP2), mRNA | |
| XM_012651 | Homo sapiens collagen, type I, alpha 1 (COL1A1), mRNA | |
| XM_015434 | Homo sapiens chitinase 3-like 1 (cartilage glycoprotein-39) (CHI3L1), mRNA | |
| XM_016181 | Homo sapiens wingless-type MMTV | |
| | integration site family, member 5A (WNT5A), mRNA | |
| XM_017096 | Homo sapiens active BCR-related gene (ABR), mRNA | |
| XM_017384 | Homo sapiens matrix metalloproteinase 7 (matrilysin, uterine) (MMP7), mRNA | Matrix metalloproteinase 7 (matrilysin, uterine) |
| XM_017591 | Homo sapiens annexin A6 (ANXA6), mRNA | Annexin VI (p68) |
| XM_028204 | Homo sapiens nuclear factor of kappa light polypeptide gene | |
| | enhancer in B-cells 1 (p105) (NFKB1), mRNA | |
| XM_028642 | Homo sapiens integrin, alpha 5 | |
| | (fibronectin receptor, alpha polypeptide) (ITGA5), mRNA | |
| XM_029245 | Homo sapiens collagen, type I, alpha 2 (COL1A2), mRNA | Collagen, type I, alpha-2 |
| XM_029796 | Homo sapiens frizzled-related protein (FRZB), mRNA | |
| XM_031221 | Homo sapiens interleukin 1, alpha (IL1A), mRNA | |
| XM_031288 | Homo sapiens aggrecan 1 (chondroitin sulfate proteoglycan 1, large | |
| | aggregating proteoglycan, antigen identified by monoclonal | |
| | antibody A0122) (AGC1), mRNA | |
| XM_031289 | Homo sapiens interleukin 8 (IL8), mRNA | |
| XM_032902 | Homo sapiens integrin, alpha 1 (ITGA1), mRNA | |
| XM_033470 | Homo sapiens defender against cell death 1 (DAD1), mRNA | DEFENDER AGAINST CELL DEATH 1 |
| XM_033657 | Homo sapiens heparan sulfate proteoglycan 2 (perlecan) (HSPG2), mRNA | |
| XM_033878 | Homo sapiens tissue inhibitor of metalloproteinase 1 (erythroid potentiating | |
| | activity, collagenase inhibitor) (TIMP1), mRNA | |
| XM_034023 | Homo sapiens regulator of G-protein signalling 4 (RGS4), mRNA | Human RGP4 mRNA, complete cds |
| XM_034556 | Homo sapiens chloride channel 7 (CLCN7), mRNA | |
| XM_034845 | Homo sapiens phosphatase and tensin homolog (mutated in multiple advanced | Phosphatase and tensin homolog |
| | cancers 1) (PTEN), mRNA | (mutated in multiple advanced cancers 1) |
| XM_034890 | Homo sapiens fibrillin 1 (Marfan syndrome) (FBN1), mRNA | |
| XM_035662 | Homo sapiens cathepsin B (CTSB), mRNA | |
| XM_035842 | Homo sapiens small inducible cytokine A5 (RANTES) (SCYA5), mRNA | Small inducible cytokine A5 (RANTES) |
| XM_036107 | Homo sapiens integrin, beta 2 (antigen CD18 (p95), lymphocyte function-associated | |
| | antigen 1; macrophage antigen 1 (mac-1) beta subunit) (ITGB2), mRNA | |
| XM_036175 | Homo sapiens collagen, type XVIII, alpha 1 (COL18A1), mRNA | Collagen, type XVIII, alpha 1 |
| XM_037087 | Homo sapiens ATP binding protein associated with cell differentiation | Homo sapiens mRNA for ATP binding |
| | (APACD), mRNA | protein, complete cds |
| XM_037646 | Homo sapiens msh homeo box homolog 2 (Drosophila) (MSX2), mRNA | |
| XM_037965 | Homo sapiens chondroadherin (CHAD), mRNA | |
| XM_038584 | Homo sapiens tissue inhibitor of metalloproteinase 3 | |
| | (Sorsby fundus dystrophy, pseudoinflammatory) (TIMP3), mRNA | |
| XM_039094 | Homo sapiens SRY (sex determining region Y)-box 9 | |
| | (campomelic dysplasia, autosomal sex-reversal) (SOX9), mRNA | |
| XM_040037 | Homo sapiens adrenergic, beta, receptor kinase 1 (ADRBK1), mRNA | Adrenergic, beta, receptor kinase 1 |
| XM_040385 | Homo sapiens S-adenosylmethionine decarboxylase 1 (AMD1), mRNA | S-adenosylmethionine decarboxylase 1 |
| XM_042153 | Homo sapiens biglycan (BGN), mRNA | Biglycan |
| XM_042664 | Homo sapiens nuclear autoantigenic sperm protein (histone-binding) (NASP), | Nuclear autoantigenic sperm |
| | mRNA | protein (histone-binding) |
| XM_044120 | Homo sapiens fibroblast growth factor receptor 3 | |
| | (achondroplasia, thanatophoric dwarfism) (FGFR3), mRNA | |
| XM_045089 | Homo sapiens ATPase, Cu++ transporting, beta polypeptide (Wilson disease) | ATPase, Cu++ transporting, |
| | (ATP7B), mRNA | beta polypeptide (Wilson disease) |
| XM_045802 | Homo sapiens paxillin (PXN), Mrna | |
| XM_045890 | Homo sapiens ADP-ribosylation factor 4-like (ARF4L), mRNA | ADP-ribosylation factor 4-like |
| XM_045925 | Homo sapiens decorin (DCN), mRNA | Decorin |
| XM_045926 | Homo sapiens lumican (LUM), mRNA | |
| XM_046035 | Homo sapiens integrin, alpha L (antigen CD11A (p180), lymphocyte function- | Integrin, alpha L (antigen |
| | associated antigen 1; alpha polypeptide) (ITGAL), mRNA | CD11A (p180), lymphocyte function-associated |
| | | antigen 1; alpha polypeptide) |
| XM_046758 | Homo sapiens tensin (TNS), mRNA | |
| XM_046765 | Homo sapiens thymidylate synthetase (TYMS), mRNA | Thymidylate synthase |
| XM_047231 | Homo sapiens fibulin 1 (FBLN1), mRNA | |
| XM_047719 | Homo sapiens transcription factor 7 (T-cell specific, HMG-box) (TCF7), mRNA | Transcription factor 7 (T-cell specific) |
| XM_047802 | Homo sapiens a disintegrin-like and metalloprotease (reprolysin type) | |
| | with thrombospondin type 1 motif, 5 (aggrecanase-2) (ADAMTS5), mRNA | |
| XM_048167 | Homo sapiens troponin T1, skeletal, slow (TNNT1), mRNA | Troponin T1, skeletal, slow |
| XM_048201 | Homo sapiens metallothionein 1L (MT1L), mRNA | Metallothionein 1L |
| XM_049177 | Homo sapiens vascular endothelial growth factor B (VEGFB), mRNA | |
| XM_049518 | Homo sapiens intercellular adhesion molecule 1 (CD54), human rhinovirus | Intercellular adhesion molecule |
| | receptor (ICAM1), mRNA | receptor 1 (CD54), human rhinovirus |
| XM_049534 | Homo sapiens amylase, alpha 2A; pancreatic (AMY2A), mRNA | Amylase, alpha 2A; pancreatic |
| XM_049690 | Homo sapiens coatomer protein complex, subunit alpha (COPA), mRNA | Human coatomer protein (HEPCOP) |
| | | mRNA, complete cds |
| XM_049864 | Homo sapiens colony stimulating factor 3 (granulocyte) (CSF3), mRNA | |
| XM_049937 | Homo sapiens insulin-like growth factor binding protein 4 (IGFBP4), mRNA | |
| XM_050846 | Homo sapiens Indian hedgehog homolog (Drosophila) (IHH), mRNA | |
| XM_053809 | Homo sapiens similar to chondroitin sulfate proteoglycan 2(versican) | |
| | (H. sapiens) (LOC153633), mRNA | |
| XM_054566 | Homo sapiens collagen, type VI, alpha 1 (COL6A1), mRNA | |
| XM_054686 | Homo sapiens caspase 3, apoptosis-related cysteine protease (CASP3), mRNA | |
| XM_055254 | Homo sapiens fibronectin 1 (FN1), mRNA | |
| XM_058069 | Homo sapiens matrix metalloproteinase 12 (macrophage elastase) (MMP12), mRNA | |
| XM_084239 | Homo sapiens retinoic acid receptor responder (tazarotene induced) 2 | Human tazarotene-induced |
| | (RARRES2), Mrna | gene 2 (TIG2) mRNA, complete cds |
| XM_084263 | Homo sapiens cytochrome c oxidase subunit VIc (COX6C), mRNA | Human mRNA for |
| | | cytochrome c oxidase subunit VIc |
| XM_084285 | Homo sapiens integral membrane protein 2A (ITM2A), mRNA | |
| XM_085705 | Homo sapiens tissue inhibitor of metalloproteinase 2 (TIMP2), mRNA | |
| XM_086368 | Homo sapiens MUF1 protein (MUF1), mRNA | H. sapiens mRNA for MUF1 protein |
| XM_096277 | Homo sapiens collagen, type V, alpha 1 (COL5A1), mRNA | |
| Y00985 | Human mRNA for manganese-containing superoxide dismutase | |
| Y07566 | H. sapiens mRNA for RIT protein | H. sapiens mRNA for RIT protein |
| Y07570 | H. sapiens mRNA for PHAPI2b protein | H. sapiens mRNA for PHAPI2b protein |
| Y08999 | H. sapiens mRNA for Sop2p-like protein | H. sapiens mRNA for Sop2p-like protein |
| Y12692 | Homo sapiens mRNA for WNT11 gene | |
| Y13936 | Homo sapiens mRNA for protein phosphatase 2C gamma | Homo sapiens mRNA for |
| | | protein phosphatase 2C gamma |
| Y15227 | Homo sapiens mRNA for leukemia associated gene 1 | Homo sapiens mRNA for |
| | | leukemia associated gene 1 |
| Z22865 | H. sapiens dermatopontin mRNA, complete CDS | |
| Z50781 | H. sapiens mRNA for leucine zipper protein | |
| Z50853 | H. sapiens mRNA for CLPP | H. sapiens mRNA for CLPP |

The current invention also encompasses the process of down compression of previously determined 467 genes to a lower number that is still able to characterize the desired number of different cellular status. At present, for the determination of 7 different cell types or development stages, a minimum of 26 spots of different marker genes are preferred, much preferred about 200 such spots. For full information, at least one spot for each of the presently 467 genes (markers) is preferred. A reduction of spot number can be of relevance e.g. if under certain conditions only a small subset of those genes listed in Tab II is required for analysis e.g. in clinical applications. This down compression can be achieved by determining the ratio of actual to target number of genes and then choosing from each cluster accordingly to the determined ratio the necessary number of genes to fulfill the requirement. This process requires to group the number of genes for each analysis of e.g. Tab. I into representative cluster familys from where representative genes can be selected. Such clusters familys can be determined as shown in FIG. 1, namely by grouping clusters together that show a similar expression pattern. For each cluster family a representative number of genes may be choosen according to the compression factor that has been defined. It can easily be seen that for larger clusters like e.g. “A” in Tab I more genes are available to select while in other clusters like e.g. “E” in Tab I less are present. At the end of the process one needs to balance the procedure in order to preserve the characteristics of the expression profile. In order to do so the amount of genes for each analysis should at least be greater than 2 sequences or spots, respectively, of different genes and for the total array at least 30. In order to control such a process classical hierarchical clustering (Stanford) analysis can be performed and checked on graphical presentations like treeview (Stanford). Cluster analysis may group similar expression profiles in families and will allow distinguishing between different cell sources and allows classification of these cell cultures (see FIG. 2). If the cell sources are not properly represented in the cluster analysis it means that the selected marker genes are not balanced.

Example of an Cartilage Specific Micro Array Structure:

To produce a microarray with printed oligonucleotides sequences of approx. at least 10 mers, preferably at least 25 mers, some sequences of table II need to be further processed. Since some of the determined sequences in Tab II are only expressed sequence tags (herein referred to as EST), they do not correspond to or represent the full-length cDNA. Therefore the EST preferably is BLAST searched with the public database at NCBI and the corresponding full-length cDNA determined. Only by having the correct and full-length cDNA it is possible to design oligomeric sequences that are balanced to each other and minimize any cross reactivity. Exemplary polynucleotide sequences (targets) are provided in the sequence listing of Table III. The cartilage related polynucleotide sequences as e.g. listed in Table III and other polynucleotide sequences known as key cartilage genes from the literature can be immobilized on a substrate and used as hybridizable array elements in a microarray format. Such microarrays can be composed of a subset of oligonucleotides representing e.g. sequences listed on Tab. II but modified to represent only full-length cDNA sequences. The used polynucleotides for the production of such a microarray can either be 50mer or also PCR (polymerase chain reaction) products but at least need to be longer then 10 bases. It should be noted that for microarray production also PCR products from the corresponding determined sequences directly or the full length cDNA can be used and it is not restricted just to oligonucleotides.

Methods to anchor such oligonucleotides or polynucleotides on a solid support are described in literature, together with information on length dependent distances between each oligo or polynucleotides and spots. (see e.g. Principal and Practice, DNA microarrays: gene expression analysis B. Jordan, Springer, 2001)

When polynucleotides are employed as hybridizable array elements in a microarray and depending on the software used, the array elements may be organized in an ordered fashion so that each element is present at a specified location on the substrate. If the array elements are at specified locations on the substrate, the hybridization patterns and intensities (which together create a unique expression profile) can be interpreted in terms of expression levels of particular genes. This expression profile can then be used and may be correlated with any effect associated with a tissue and/or compound or to be investigated with regard to a specific tissue and/or compound and allows comparison with already existing data.

One of such useful application of using ordered polynucleotides on microarrays is e.g. the comparison of gene expression profiles from a new sample e.g. a tissue biopsy, with already determined characteristic gene expression profiles that are preferably stored in a database. Such stored gene expression profiles are e.g. of major importance if microarrays are applied in the clinic. In this case advantageously a database is set up that stores the corresponding gene expression profiles and advantageously also all patient informations, e.g. history, blood pressure etc. By including all patient data and gene expression profiles in the analysis process and then starting a comparison with an expression profile from a new biopsy, it becomes possible to achieve a stronger correlation with the clinical outcome. This will allow to determine which therapy shall be applied, or even to modify an existing therapy, e.g. to add growth factor x at a concentration y during the ex vivo tissue engineering phase. It may also be the case that the biopsy sample will demonstrate a poor gene expression profile that precludes the successful application of a modern therapeutic cell/tissue approach. Such cases would then only qualify for traditional surgical approaches, and hence would not obtain the benefits of the tissue engineering process.

In analogy, the assessment of in vitro produced cartilage can also be performed. In the same way as mentioned above cell culture parameters, like e.g. culture media conditions, growth factor concentration, are preferably stored in a data base together with the corresponding gene expression profiles. Comparison of the database entry with new profiles of new samples can then be used to assess the quality of the new in vitro produced tissue.

Subject Arrays and Their Use:

It should be noted that the invention described here is not dependent on any special array format rather than the possibility to select from an extended list of 467 novel key cartilage genes as well as meaningful gene expression patterns. A presently preferred subject array is a novel cartilage specific microarray that includes 187 genes that in the scope of this invention have been determined to be cartilage related and 140 genes that have been connected to cartilage in literature (see also Tab II). Normally, in high-density array procedures up to 10000 genes are usually applied and are not specific for certain applications. As one major general drawback, this results in massive data overflow and impaired data analysis due to difficult data handling and procedures. A preferred array has in its current state a minimal number of 150 genes, presently much preferred at most 333 genes, all of those with demonstrated relevance within cartilage tissue. Another major limitation has become apparent. While the invention WO01/24833 A2 describes a few marker genes associated with cartilage phenotype stability they do not allo w to extensively describe chondrocyte cultures in details. No comprehensive classification of the different cell populations and culture conditions is possible as well as no gene expression profile or fingerprint can be achieved. Gene expression profiles determined with a set of genes represented in Tab II may allow to perform a more comprehensive analysis of different cell cultures conditions. Furthermore it may allow to compare and classify different tissue or the result of the different applied cell culture conditions. The above mentioned topics may only be possible with the disclosed invention as outlined within the following applications.

The inventive array CART-CHIP 300 ™ may be applied to classify (quality control) any source material, such as human cartilage biopsies, mesenchymal stem cell containing bone marrow aspirate, or pre-chondrogenic cells containing tissue according to pre-defined categories with respect to their capacity to re-build or re-organize a hyaline cartilage-like matrix in vitro. A rough subdivision could be for example “A”, “B”, or “C”. While “A” will easily produce cartilage-like matrix, “B” will require special treatment to achieve an implantable construct, and “C” will represent those cases that do yet not qualify for such a procedure. This biopsy classification system will allow:
Quality control of the starting biopsy material and therefore optimization of the downstream process regarding e.g. in vitro tissue engineering applications
Diagnostic evaluation of the patient and candidate treatment methods (e.g. CARTIGRAF™) to ensure a cost-optimized procedure
Quality control of in vitro tissue engineered products

The subject array of the present invention can be employed for all kind of research and developmental studies related to in vitro tissue engineering of cartilage. The possibility to assess proliferation, differentiation or re-differentiation as well as de novo matrix formation processes through analyses and comparison of a plurality of key cartilage genes (positive/negative markers) within one single experiment replaces current trial and error approaches and is thus far more rational.

The subject array can be applied to screen all kind of drugs, e.g. hormones, growth factors, within in vitro chondrocyte cultures regarding a potential beneficial effect on proliferation, differentiation, de novo matrix formation. The deduced expression profiles can then be compared with existing data of e.g. native cartilage tissue and used to further optimize the process. Additionally the expression profiles can be compared with data from human adult and human infant cartilage to deduce a pathway or a strategy of how to induce more tissue formation in vitro.

The subject array of the preferred embodiment is very well suitable to better understand reaction pathways leading to new responses of chondrocytes in vitro. Only key cartilage genes comprising the whole spectrum of functional gene categories are to be investigated. This can be used to study the complexity of degenerative cartilage process in vitro and the respective influence of potential beneficial drugs.

The subject array may be used to optimize cultures for in vitro cartilage formation starting from human cell sources other than cartilage like e.g. mesenchymal stem cells or bon marrow aspirates.

This subject array will be preferably used as powerful alternative for conventional molecular biology tools beside more established histological and biochemical analyses. By focusing on the most prominent cartilage marker genes being either positive or negative, it is possible to characterize cartilage or cartilage related tissues as well as cell cultures thereof. In this respect, the subject array can replace conventional RT-PCR studies performed to check for cartilage marker gene expression, e.g. collagen I versus collagen II, aggrecan versus versican. By applying this subject array the set of markers will be easily increased by simultaneously simplifying the experimental procedure and enhancing the outcome.

The subject arrays of the present invention have several advantages compared to existing microarrays as well as to conventional gene expression tools such as RT-PCR, Northern Blots etc.

Most importantly, the subject arrays are all based on key cartilage genes. Beyond all the key cartilage genes known from the literature (˜100-200 genes), 467 additional cartilage relevant genes have been discovered. Thus a significantly increased pool of cartilage key genes exists to choose from for various applications. For instance, to understand degenerative processes as they occur in OA or RA by study of complex biological reaction pathways, it is important to follow expression of a relatively large number of genes.

### EXAMPLES

The examples are described for the purposes of illustration and are not intended to limit the scope of the invention.

### Example 1

### Analysis of Various Human Cartilage Samples

Useful for characterizing chondrocyte cultures derived from different human cartilage samples (adult and fetal), where adult samples are different with respect to their capacity to form living tissue engineered equivalents under high density culture conditions.

Adult chondrocytes show different gene expression clusters compared to fetal chondrocytes and can be further distinguished from samples that will not produce living cartilage constructs (failures).

Human chondrocytes from adult and fetal articular cartilage were proliferated in DMEM-F12 medium containing 10% FCS over several passages and transferred to pellet cultures (0.5*10⁶ cells) in serum free DMEM-F12 medium supplemented with Ascorbate and Insulin medium. Proliferated cells were directly lyzed with RLT buffer (RNeasy® Mini Kit, Qiagen) after trypsin release from plastic substrate, shredded (QIAshredder, Qiagen) and kept frozen at −80° C. in lysis buffer for later processing. High density pellet cultures were cultivated for 2 weeks if not otherwise specified, subsequently washed with phosphate buffered saline (PBS) and lyzed in RLT Buffer (supplied with RNeasy® Kit). Total RNA was isolated from all samples as described in the manual provided with the RNeasy kit and stored at −80° C.

Fluorescent labeled aRNA (amplified RNA) constructs were obtained by in vitro reverse transcription of the RNA followed by an in vitro amplification reaction.

2 μg of isolated total RNA were used per sample to amplify RNA by applying only one cycle of in vitro transcription (IVT, Millenium Biologix AG, Application Note).

2 μg of total RNA from each sample was primed with oligo(dT)₂₄₋ₘₑᵣ (containing a T7 RNA Polymerase Promotor) and reverse transcribed using 400 Units SuperScript II reverse transcriptase enzyme, nucleotides, 5× Reaction Buffer and Dithiothreithol (DTT) as described in protocol provided with the enzyme. For ribonuclease protection 1 μL RNase inhibitor (10 Units) was used to prevent RNA degradation during first strand synthesis. This first strand synthesis reaction was incubated for 1 hour at 42° C.

To the first strand synthesis reaction 93 μl nuclease free water, 30 μl second strand buffer (Invitrogen, Basel, Switzerland) and 1.5 μl nuclotide mix (dATP, dTTP, dGTP, dCTP, 25 mM each) was added.

Second strand synthesis reaction mix was obtained by adding 40 Units E. coli polymerase I (New England Biolabs, BioConcept, Allschwil, Switzerland), 10 Units E. coli DNA Ligase (New England Biolabs, BioConcept, Allschwil, Switzerland) and 2.5 Units Ribonuclease H (Fermentas, Labforce AG, Nunningen, Switzerland). Reaction was incubated for 2 hours at 16° C.

After this incubation step remaining RNA was degraded by adding 7.5 μl 1M sodium hydroxid containing 2 mM EDTA (Ethylenediaminetetraacetic acid) for 10 minutes at 65° C. 7.5 μl 1M Hydrochloric acid was added to neutralize the reaction.

The obtained double strand DNA was purified in a QIAquick® PCR purification kit (Qiagen, Hilden, Germany) and concentrated to 7.5 μl. To this concentrated RNA following reagents were added to obtain aRNA synthesis mix: 2 μl ATP (Adenosine triphosphate, 75 mM), 2 μl GTP (Cytidin triphosphate, 75 mM), 2 μl GTP (Guanosin triphosphate, 75 mM), 2 μl UTP (Uridin triphosphate, 75 mM), 1.5 μl 5-(3-aminoallyl)-Uridin triphosphate and 2 μ, reaction buffer and 2 μl Enzyme mix (both provided with Ambion MegaScript Kit, Ambion, Cambridgeshire, United Kingdom).

This aRNA synthesis mix was incubated for 4 hours at 37° C. Remaining double strand DNA was digested by adding 1 μl Dnase I for 15 min at 37° C. aRNA was cleaned and concentrated with an RNeasy® Mini Kit column (Qiagen, Hilden Germany) and then concentrated to a final volume of 9 μl.

Fluorescent dye molecules were coupled to the reactive aminoallyl groups of the incorporated a 5-(3-aminoallyl)-Uridin triphosphate molecules. One aliquot of either Cy3™- or Cy5™-mono reactive dye (Amersham Biosciences, Buckinghamshire, United Kingdom) was diluted in 40 μl water free Dimethyl sulfonoxide. 10 μl, of one of the diluted Cy™ mono reactive dyes was added to each sample buffered in 100 mM Carbonate buffer (pH 9.00). Reaction was quenched after 1 hour by adding 10.4 μl Ethanol amine for 15 min at room temperature.

Unincorporated dye molecules were removed by ethanol precipitation. 2 μl Glycogen (Invitrogen, Basel, Switzerland) was added as carrier during precipitation. After precipitation aRNA pellet was washed with 80% ethanol, dried and resuspended in 50 μl 1× Fragmentation buffer (200 mM Tris(hydroxymethyl)aminomethane hydrochloride, 500 mM Potassium acetate, 150 mM Magnesium acetate). aRNA was fragmented for 35 min at 94° C. and placed on ice immediately. Fragmented aRNA was dissolved in 900 μl hybridization buffer.

For denaturation aRNA was incubated for 5 min at 98° C. and centrifuged for 30 sec at full speed in a microcentrifuge.

One CART-CHIP™ 300 (Millenium Biologix AG, Switzerland) was placed face down in a standard hybridization chamber. Hybridization solution containing the denatured and labeled aRNA sample was injected using a standard micropipet whereas Cy3™ and Cy5™ samples were hybridized together in one hybridization chamber (Millenium Biologix AG, Switzerland). The microarrays were incubated overnight at 42° C. in a PCR thermal cycler (TGradient, Whatman Biometra GmbH, Göttingen, Germany).

After incubation unspecific aRNA probe was washed away with 1×SSC, 0.1% SDS for 5 min at room temperature, followed by another wash step in 1×SSC, 0.1% SDS for 5 min and rinsed with 1×SSC without SDS for 1 minute to remove excessive SDS. 1×SSC was discarded. Remaining 1×SSC buffer on the slide surface was removed by centrifuge the slide for 2 min at 1500×g.

The dried CART-CHIP™ 300 were then scanned using an Affymetrix 418 microarray scanner.

Expression level raw data for every spot was obtained with ImageQuaNT (Molecular Dynamics). Raw data was normalized by dividing every expression value by total expression value of all spots for every sample and filtered by setting all values below the 25 percentile to the value of this 25 percentile to remove noise (25 percentile threshold).

For each sample (e.g. de-differentiated and re-differentiated chondorcytes) a list of all measured genes was generated. This so called gene expression profile was then used for subsequent analyses.

Further data analysis was performed using either hierarchical clustering with cluster.exe (written by Michael Eisen, Stanford University) or Self Organizing Maps (SOM), such as GeneCluster developed by Whitehead Institute (Massachusetts Institute of Technology, MIT). The settings of the software were optimized until a reasonable number of clusters resulted that were able to represent the comparison thoroughly. In the following example the parameters were as following:

Basic parameters: SOM rows 6; SOM col: 4; #epochs=3000; #seeds=1

Advanced parameters: initialization: random vectors; neighborhood: bubble; alpha 1=2; alpha f=0.005; sigma 1=3000; sigma f=2.

FIG. 1 shows a typical result from a SOM analysis with the above mentioned basic parameters, whereas FIG. 2 shows an example of a graphical presentation of a cluster analysis and viewed by the software treeview.

### Example 2

### Quality Control and Human Cartilage Sample Classification

Useful to demonstrate how CART-CHIP™ 300 can be used to differentiate between diverse cell culture conditions, to distinguish different patients, to study the influence of 3D culture conditions and to serve as a quality control tool during any tissue engineering process.

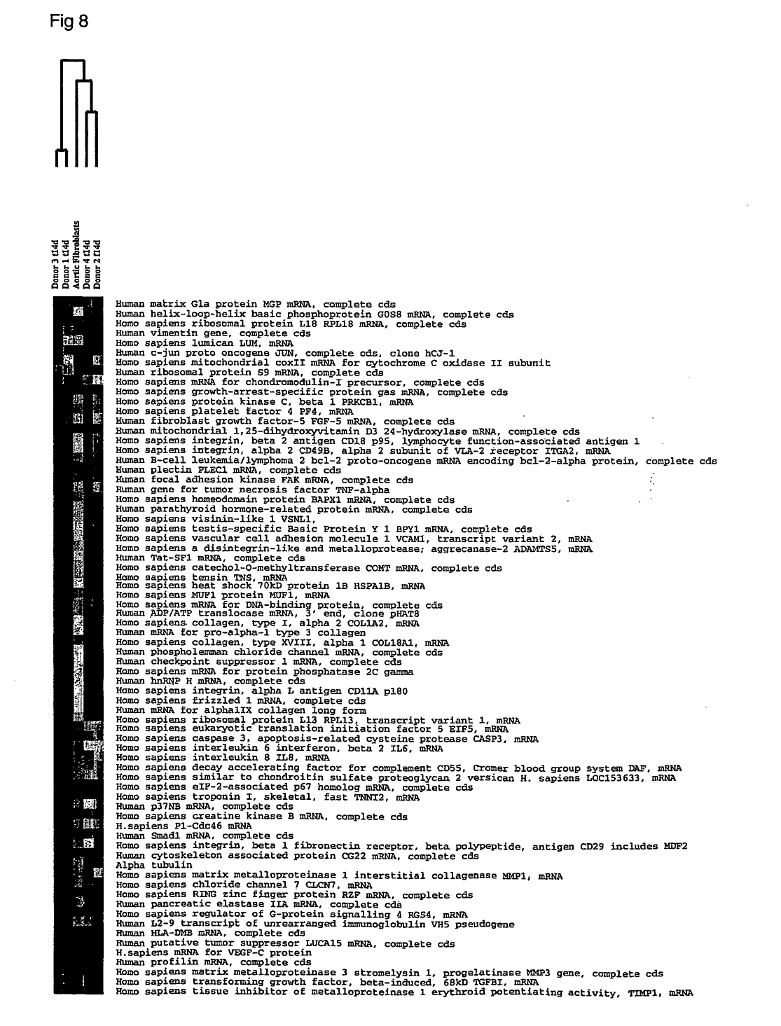

Human chondrocytes isolated from 4 different donors were proliferated over one passage (P1) and then cultivated as high density pellets (0.5*10⁶ cells) in 3D culture for 7 and 14 days. RNA samples were taken from proliferated as well as from 3D cultured cells resulting in totally 12 different samples as shown in FIG. 8. RNA isolated from this samples was shreddered in a QIAshredder (QIAGEN, Hilden, Germany), amplified, hybridized, washed and scanned as described in Example 1.

Data sets for all 12 samples were extracted and normalized as described in Example 1 to perform cluster and SOM analysis as noted below. Cluster analysis was performed using normalized data computed with GeneCluster.

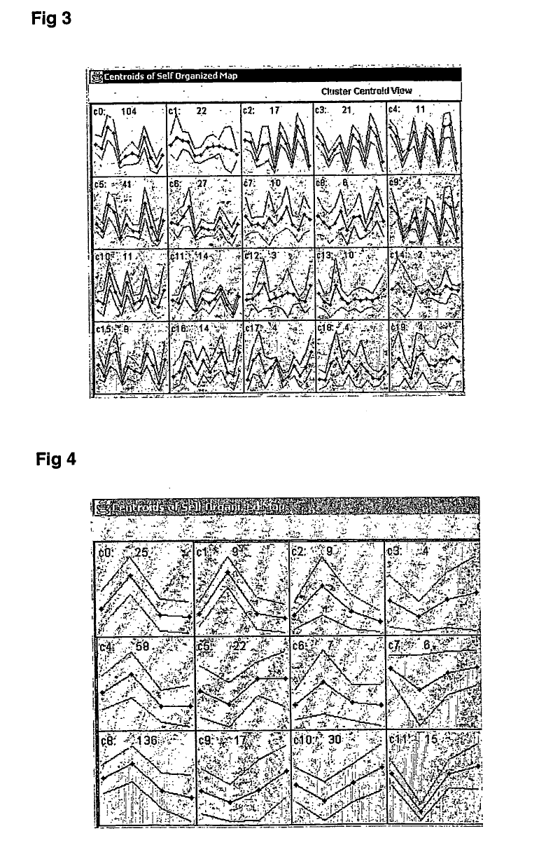

FIG. 3 shows a picture of such a cluster analysis for all 12 samples (#1-#12) consisting of 20 clusters (c0-c19)

Every cluster represents a typical gene expression pattern for all 12 samples indicated by a point, starting from sample #1 on the left hand side to sample #12 on the right hand side in every cluster. For example cluster c0 represents the expression level of 104 genes in all 12 samples in a given range indicated by the lines located above and below the computed points.

Another example for gene expression levels that behave similar for different culture conditions and donors are depicted in clusters c3, c4, c9 and c10. Meaning that every subset of the three donor specific points #143, #4-#6, #7-#9, #10-#12 (see Tab V for detailed description) have gene clusters that behave similar in all analyzed samples.

An example of differently behaving genes is indicated in cluster c13, representing 10 genes that behave similar in donor #1 and #2 but show a different gene expression patterns for donors #3 and #4.

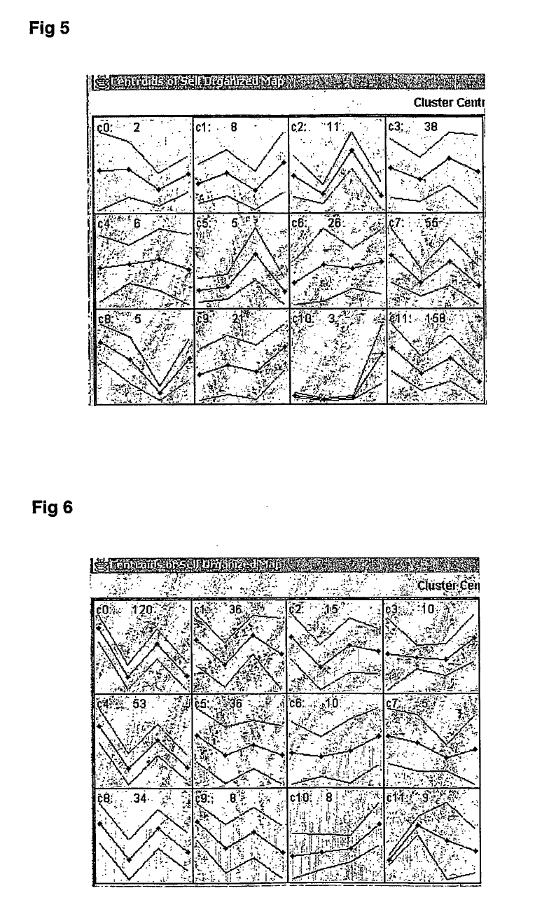

More detailed analyses are shown in FIG. 4, FIG. 5, and FIG. 6. The clusters produced in these figures clearly demonstrate differences as well as similarities in cell behavior for either t0, t7 or t14 days, respectively.

Another software algorithm that can be applied for analysis of large amounts of data coming from gene microarrays is called hierarchical cluster analysis, whereas genes and/or different conditions with similar behavior in gene expression are clustered together. All hierarchical cluster analyses were performed using Cluster software described in Eisen et al. (1998) PNAS 95:14863) and displayed using treeview.exe developed by same author.

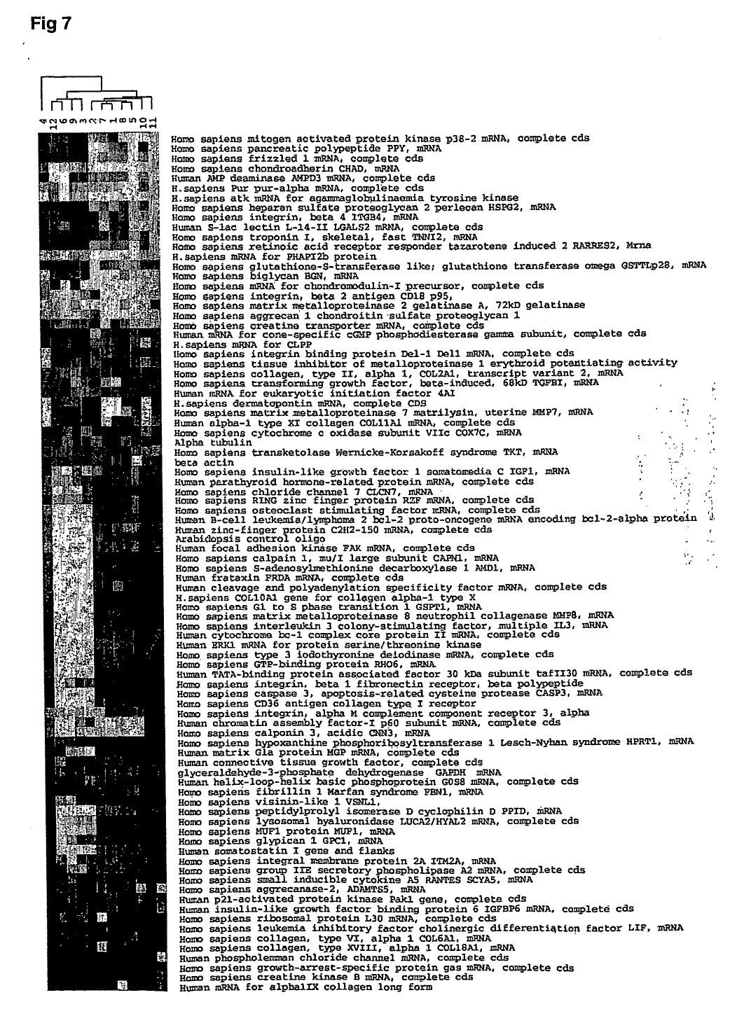

FIG. 7 shows such a cluster of selected genes for all 12 samples analyzed. Every square is representing one single gene expression value. Different intensity means different expression levels. Dark squares are representing samples without any significant change in gene expression compare to the other samples or patients. Bright squares are indicating samples in which genes are up- or down-regulated relative to other samples analyzed. A so called cluster of genes is a group of genes that behave similar from one donor to the other donors.

Not only genes but also samples can be clustered together. These clusters are called similarity dendrograms, shown in the top part of FIG. 7. These tree-like structures illustrates similarities in gene expression between different samples or donors. The closer a sample (#1 . . . #12) is located to another sample in this dendrogram the more similar gene pattern they have.

Interestingly to see is that the seven samples located at the right side of the dendrogram (samples #1, #2, #5, #7, #8, #10 and #11) are clustered together. This samples are representing t0 and t7 conditions as described above (illustrated in Tab V), whereas a cluster of 4 samples in the middle of the dendrogram (samples #3, #6, #9 and #12) are representing only t14 samples. This means a microarray of the current invention is able to distinguish between de-differentiated, proliferated samples (t0 and t7) and re-differentiated samples in a later stage (t14).

An outlier represents sample #4 located at the most left side of FIG. 7. which represents proliferated chondrocytes (t0) from donor 2 and could not clustered together with the remaining proliferated samples. Interestingly, this sample that it is not similar to all other proliferated samples (#1, #7 and #10) was impaired with its capacity to form cartilage tissue equivalents following expandation in 2D culture. The biochemical analysis revealed a lower amount of total collagen/DNA for this sample and immunohistochemisty with collagen II antibodies resulted in only weak staining for a collagen II.

### Example 3

### Aortic Fibroblasts vs. Chondrocytes

Example to differentiate between expanded chondrocytes and aortic fibroblasts cultivated over 14 days in 3D settings.

A human aortic fibroblast cell source was proliferated and brought to 3D culture. RNA was isolated after 14 days of culture. Expression data analysis was performed as described in previous Examples 1 and 2 using CART-CHIP™ 300 microarray.

A hierarchical cluster analysis was performed as described in example 2. Samples representing 3D culture after 14 days (t14) were included in said data analysis (samples #3, # 6, # 9 and #12, see Tab V).

The result of the described analysis can be seen in FIG. 8. The upper part of the figure shows a dendrogram as described in example 2. Aortic fibroblasts are not clustered together with human chondrocytes. The cluster shows a significantly different pattern compared to all other cultures.

Obviously a gene expression pattern of an aortic fibroblast cell source can be clearly separated from a gene expression pattern of human chondrocytes. A micorarray of the present invention is therefore not only able to study differences between different chondrocyte culture conditions but also to distinguish between cells isolated from different tissues.

### Example 4

### Arthritic Conditions vs. Healthy Conditions

Useful to distinguish between normal healthy chondrocyte behavior from cells resembling an arthritic phenotype. Interleukin-1β is known to play a central role in the inflammation and connective tissue destruction observed in both rheumatoid arthritis (RA) and osteoarthitis (OA). Stimulation of in vitro chondrocyte cultures with Interleukin-1β thus represents a simple experimental arthritis model.

The chondrocyte cell source from donor 4 (see Tab V) was proliferated over 3 passages and then cultivated as high-density pellet cultures (0.5*10⁶ cells) for 16 hours and 7 days either in the absence or presence of Interleukin-1β (30 ng/mL). RNA was isolated from all samples, hybridized to CART-CHIP™ 300 and expression profiles were generated as described in Example 1.

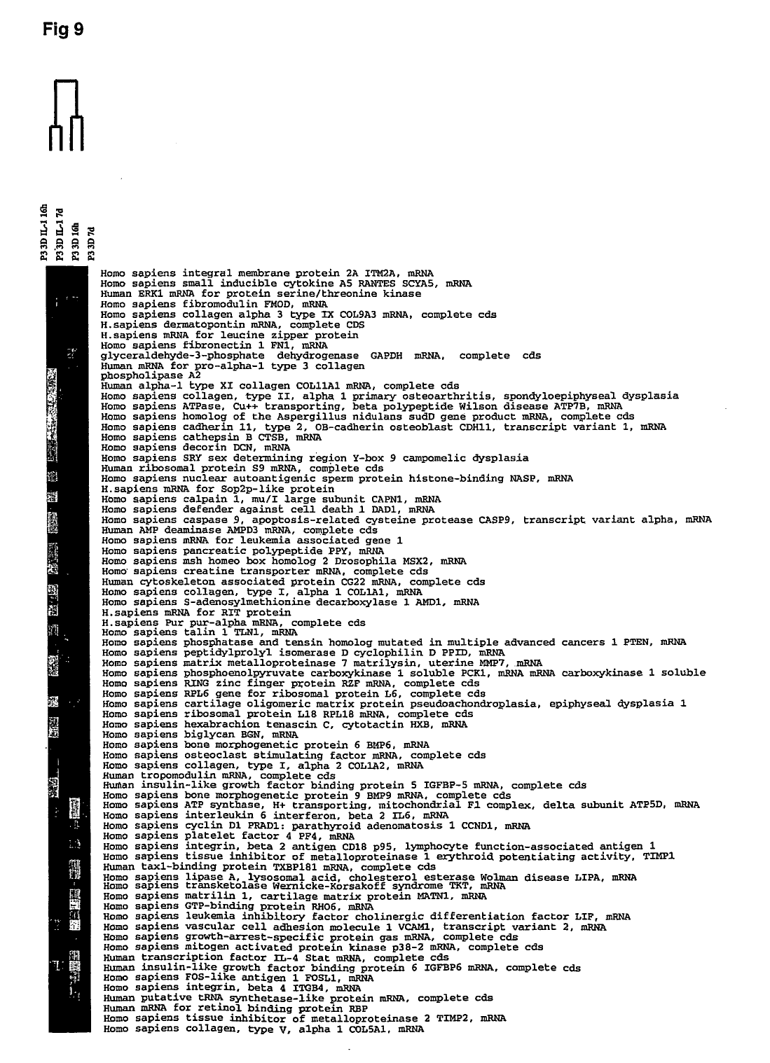

A hierarchical cluster analysis was performed as described in Example 1 and the dendrogram and a selection of the representative gene clusters are shown in FIG. 9. This clearly shows that already a short stimulus of Interleukin-1β results in alteration of the chondrocyte phenotype with gene expression changes that can be distinguished from untreated normal chondrocyte cultures.

Appendix

TABLE I

| | Number of marker | |
|---|---|---|
| Experiment and correlated gene | genes for each | experiment |
| expression analysis | experiment (analysis) | (analysis) |
| 2D marker adult vs. fetal/infant | 151 | A |
| 2D/3D adult vs. fetal/infant | 96 | B |
| 3D marker failure | 165 | C |
| 3D marker adult/fetal/infant | 350 | D |
| 2D/3D marker adult | 48 | E |
| Time dependent failure marker | 75 | F |
| 3D failure marker | 41 | G |
| Apoptosis related failure markers | 30 | H |

TABLE V
Sample Numbers (#)

| | Proliferation | 7 days in 3D culture | 14 days in 3D culture |
|---|---|---|---|
| | (t0) | (t7) | (t14) |
| Donor 1 | #1 | #2 | #3 |
| Donor 2 | #4 | #5 | #6 |
| Donor 3 | #7 | #8 | #9 |
| Donor 4 | #10 | #11 | #12 |

Table IV shows the results of a bioinformatic analysis of gene expression profiles of the 467 cartilage specific marker genes.

| | Clus- | | | | | | | |
|---|---|---|---|---|---|---|---|---|
| Accesion | ter | SOM Description | H4 P2 3D | failure pool | H5 P1 2D | H4 P2 2D | H4 P1 2D | H5 P1 3D |
| AA283693 | 2 | 2D/3D | 0.620146952 | 0.563012608 | 0.543651567 | 0.467462855 | 0.502962907 | 0.971864748 |
| AA845156 | 2 | 2D/3D | 1.822887384 | 1.974062517 | 1.793508407 | 2.056234156 | 2.134502741 | 2.109619189 |
| R52548 | 22 | 2D/3D | 42.15305285 | 24.91632836 | 36.03204148 | 38.20234471 | 38.12886555 | 36.73272848 |
| T67128 | 4 | 2D/3D | 0.114907897 | 0.165223543 | 0.175143838 | 0.230515232 | 0.111518632 | 0.115949318 |
| AA845015 | 8 | 2D/3D | 5.152985898 | 5.163366386 | 4.600617698 | 5.490400618 | 5.613170246 | 5.081143315 |
| AA937895 | 8 | 2D/3D | 2.841572293 | 2.035680155 | 2.574530008 | 3.532376413 | 2.904240444 | 2.88987266 |
| AA844998 | 15 | 2D/3D | 9.597114182 | 13.63928438 | 14.05976269 | 12.99542024 | 10.56590581 | 11.73420854 |
| AA844518 | 16 | 2D/3D | 34.95251239 | 32.56636092 | 35.29612046 | 39.31305252 | 38.10953207 | 32.87879549 |
| AA894557 | 16 | 2D/3D | 56.23673424 | 43.61031652 | 36.30704958 | 58.47875881 | 59.70042623 | 48.33981986 |
| AA872001 | 20 | 2D/3D | 81.81389212 | 56.44454367 | 38.30704958 | 89.11888026 | 82.12028621 | 54.71499842 |
| H09590 | 1 | 2D/3D | 0.538758103 | 0.606028834 | 0.923090371 | 0.610970303 | 0.582446119 | 0.488768664 |
| AA888278 | 11 | 2D/3D failure vs. | 0.872435407 | 1.541743481 | 1.320841185 | 1.418331235 | 1.44054309 | 1.142574932 |
| | | cartilage | | | | | | |
| AA490855 | 11 | 2D/3D failure vs. | 5.256355042 | 3.982907846 | 3.740473099 | 5.019900855 | 5.072810576 | 4.193769533 |
| | | cartilage | | | | | | |
| H05820 | 2 | 2D/3D failure vs. | 0.326771454 | 0.298568027 | 0.723972051 | 0.373421152 | 0.291228161 | 0.42426813 |
| | | cartilage | | | | | | |
| N57766 | 3 | 2D/3D failure vs. | 1.244580679 | 1.430645893 | 1.237128762 | 1.435760892 | 1.621084111 | 1.793426963 |
| | | cartilage | | | | | | |
| AA873885 | 3 | 2D/3D failure vs. | 2.389977721 | 1.402865274 | 1.960300849 | 2.6421261823 | 1.174416144 | 2.132327967 |
| | | cartilage | | | | | | |
| AA878880 | 3 | 2D/3D failure vs. | 1.975516413 | 0.706271534 | 2.235200516 | 2.852909562 | 2.341957596 | 2.325462095 |
| | | cartilage | | | | | | |
| R54818 | 6 | 2D/3D failure vs. | 1.433222617 | 1.408764041 | 1.026467036 | 1.653245959 | 1.555280568 | 1.313068482 |
| | | cartilage | | | | | | |
| AA458830 | 23 | 2D/3D failure vs. | 6.225911646 | 7.131102869 | 6.449691069 | 7.181223742 | 7.435528281 | 8.273231774 |
| | | cartilage | | | | | | |
| W37884 | 11 | 2D/3D failure vs. | 3.42542852 | 3.434948923 | 2.822843216 | 3.920408386 | 4.031440051 | 2.924316539 |
| | | cartilage | | | | | | |
| N63192 | 11 | 2D/3D failure vs. | 2.016416698 | 1.880341817 | 1.955128455 | 2.604284112 | 2.555986174 | 1.868963757 |
| | | cartilage | | | | | | |
| R55789 | 11 | 2D/3D failure vs. | 3.050734207 | 2.916359387 | 2.459270284 | 3.086180697 | 2.768681643 | 2.87080332 |
| | | cartilage | | | | | | |
| R56871 | 14 | 2D/3D failure vs. | 8.05304356 | 6.763218112 | 9.202602512 | 9.315742803 | 9.656817425 | 6.703182113 |
| | | cartilage | | | | | | |
| AA448659 | 15 | 2D/3D failure vs. | 4.086696472 | 7.404785733 | 3.70432959 | 4.624265635 | 4.780951569 | 6.12097414 |
| | | cartilage | | | | | | |
| AA235388 | 11 | 2D/3D failure vs. | 4.793907209 | 5.590616298 | 6.430749719 | 6.119406496 | 5.787215762 | 5.448903875 |
| | | cartilage | | | | | | |
| W37769 | 18 | 2D/3D failure (adult) | 9.666153573 | 9.041797201 | 10.48060069 | 9.18235887 | 9.779057041 | 9.301866835 |
| AA421701 | 18 | 2D/3D failure (adult) | 6.39851543 | 6.944977864 | 6.787000717 | 7.805796465 | 8.044148563 | 6.091762141 |
| NB1029 | 22 | 2D/3D failure (adult) | 11.25612666 | 7.536201205 | 9.791822816 | 10.26909779 | 11.09189885 | 11.24056884 |
| AA644128 | 22 | 2D/3D failure (adult) | 8.87008786 | 8.634238881 | 8.980680688 | 10.62210945 | 11.07634385 | 8.151499526 |
| N26536 | 23 | 2D/3D failure (adult) | 1.127588919 | 0.934050212 | 1.276862277 | 1.339090617 | 1.722862887 | 1.543708239 |
| AA890663 | 23 | 2D/3D failure (adult) | 7.502466565 | 6.586516448 | 8.707110583 | 7.582524442 | 6.272458865 | 6.92700675 |
| AA405987 | 17 | 2D/3D failure (adult) | 8.007848934 | 7.398131991 | 7.180024565 | 9.644484085 | 9.329435489 | 7.017060375 |
| AA888182 | 4 | 2D/3D failure (adult) | 1.159709394 | 0.997393508 | 1.761866087 | 1.609558388 | 3.076463138 | 1.864862168 |
| H09730 | 23 | 2D/3D failure (adult) | 6.016598561 | 4.38097919 | 4.512218706 | 5.75467456 | 5.708409303 | 4.568792922 |
| AA285155 | 22 | 2D/3D failure (adult) | 3.911163963 | 4.375731694 | 4.103404996 | 3.976701687 | 3.566916248 | 4.846020855 |
| AA873351 | 1 | 2D/3D failure (adult) | 0.715716774 | 0.448961688 | 1.143094938 | 1.086305702 | 0.6643881 | 0.576661574 |
| H12320 | 1 | 2D/3D failure (adult) | 0.561973556 | 2.57723008 | 0.516528265 | 0.364969035 | 0.426769958 | 1.536698149 |
| AA856558 | 1 | 2D/3D failure (adult) | 2.083875002 | 0.734906688 | 0.98056715 | 1.17363108 | 0.340692619 | 1.146139751 |
| R43581 | 2 | 2D/3D failure (adult) | 2.106534421 | 0.601495831 | 2.998509555 | 1.885349164 | 1.78560062 | 1.913887067 |
| AA633768 | 2 | 2D/3D failure (adult) | 0.641660349 | 0.394897957 | 0.989161585 | 0.80349418 | 0.676931906 | 0.471526265 |
| AA496880 | 2 | 2D/3D failure (adult) | 0.819001028 | 0.446244605 | 1.078973938 | 0.96946743 | 1.001567705 | 0.612915544 |
| AA625632 | 4 | 2D/3D failure (adult) | 1.493144759 | 0.535655307 | 2.320048835 | 2.145585116 | 1.461514048 | 1.208574247 |
| R40850 | 3 | 2D/3D failure (adult) | 0.682308084 | 0.595008017 | 1.325881758 | 1.168113039 | 0.576613768 | 0.894548353 |
| AA486072 | 9 | 2D/3D failure (adult) | 2.033091331 | 6.009395549 | 1.397241773 | 1.306335917 | 1.667598386 | 4.321686833 |
| N80129 | 4 | 2D/3D failure (adult) | 0.351536161 | 0.860381999 | 1.012366932 | 0.336466005 | 0.442915198 | 0.530986466 |
| T67270 | 5 | 2D/3D failure (adult) | 1.963529863 | 1.201108371 | 3.314347202 | 3.108011309 | 2.159811403 | 1.603756559 |
| AA776304 | 6 | 2D/3D failure (adult) | 0.862462885 | 0.468437574 | 1.000125084 | 1.050963409 | 0.840547206 | 0.712318096 |
| AA64743 | 6 | 2D/3D failure (adult) | 1.419135024 | 0.972417085 | 1.72467226 | 1.536612587 | 1.441826888 | 1.267977482 |
| AA663983 | 7 | 2D/3D failure (adult) | 0.992237246 | 0.562108496 | 2.11407474 | 1.505738573 | 1.504937007 | 1.363338621 |
| AA634008 | 8 | 2D/3D failure (adult) | 1.633316095 | 0.546444823 | 1.670198138 | 2.668127971 | 1.579545189 | 1.258079751 |
| AA683050 | 8 | 2D/3D failure (adult) | 1.604349305 | 0.609305829 | 2.869733356 | 2.49429974 | 1.663928936 | 1.276592262 |
| AA775874 | 3 | 2D/3D failure (adult) | 1.787634446 | 0.639410548 | 1.3560157 | 1.263003994 | 1.234797131 | 2.355664777 |
| AA029934 | 19 | 2D/3D failure (adult) | 2.356636943 | 1.558846586 | 2.787420472 | 2.743130089 | 2.330296295 | 2.141155369 |
| AA872397 | 21 | 3D adult vs. fetal | 19.50233862 | 17.51369555 | 30.1499536 | 23.26630505 | 24.52945535 | 17.84127943 |
| AA428195 | 3 | 3D adult vs. fetal | 7.071920614 | 5.267967863 | 6.138557001 | 7.23507727 | 7.48271081 | 5.35099208 |
| AA478724 | 3 | 3D adult vs. fetal | 5.248364716 | 3.470387154 | 4.172040349 | 5.052705318 | 5.168529149 | 6.015264941 |
| T40541 | 2 | 3D adult vs. fetal | 3.238889946 | 12.31479553 | 2.521870024 | 1.484626573 | 2.463205882 | 6.610009772 |
| N33214 | 2 | 3D adult vs. fetal | 2.65473448 | 2.961197481 | 1.727113613 | 1.517196864 | 1.863643598 | 5.009412045 |
| W69399 | 2 | 3D adult vs. fetal | 2.836334543 | 2.418647835 | 2.384859773 | 1.271557098 | 2.218662035 | 4.984390107 |
| H85454 | 2 | 3D adult vs. fetal | 2.580688668 | 1.88582991 | 1.388608005 | 1.244844015 | 1.534483051 | 3.983108173 |
| T71284 | 2 | 3D adult vs. fetal | 1.986879018 | 2.475258573 | 0.744183047 | 0.768307025 | 0.765085426 | 4.427870608 |
| N95418 | 2 | 3D adult vs. fetal | 3.21585913 | 1.583715156 | 1.572090076 | 1.152454683 | 1.55045145 | 4.188689886 |
| AA430675 | 18 | 3D adult vs. fetal | 20.40011187 | 13.5115773 | 23.35924913 | 19.03556124 | 22.70504494 | 16.6788312 |
| AA682851 | 21 | 3D adult vs. fetal | 25.44236224 | 17.10952218 | 33.76684841 | 28.22129217 | 26.86052023 | 24.00871315 |
| AA427433 | 3 | 3D adult vs. fetal | 6.682930727 | 4.737477088 | 5.985736157 | 7.06879228 | 7.217223212 | 6.130314027 |
| AA100296 | 21 | 3D adult vs. fetal | 16.73518464 | 14.16222538 | 25.17519653 | 21.63898477 | 21.16013508 | 17.85039728 |
| AA070997 | 20 | 3D adult vs. fetal | 21.13329064 | 16.5080222 | 30.63855114 | 26.1438582 | 26.27182686 | 19.46457951 |
| R27585 | 20 | 3D adult vs. fetal | 18.95998765 | 13.51396253 | 26.82697423 | 21.45047839 | 21.44832633 | 16.96978325 |
| N71628 | 20 | 3D adult vs. fetal | 18.60838994 | 16.33393806 | 26.68120529 | 21.6113028 | 21.33877846 | 16.98487296 |
| AA484566 | 20 | 3D adult vs. fetal | 16.06992797 | 10.85633998 | 18.70563179 | 17.27026288 | 17.70073984 | 14.07448353 |
| AA043228 | 20 | 3D adult vs. fetal | 12.81312824 | 9.803849829 | 17.97620829 | 14.19332756 | 14.28559054 | 10.85546077 |
| AA478273 | 19 | 3D adult vs. fetal | 52.06929197 | 32.17017287 | 36.30704958 | 47.93432102 | 43.94005516 | 45.65265571 |
| H05618 | 2 | 3D adult vs. fetal | 2.632906988 | 4.642300363 | 1.284880525 | 0.973906438 | 1.326880261 | 4.570563698 |
| AA405562 | 5 | 3D adult vs. fetal | 6.993441389 | 4.782974749 | 5.219581264 | 6.611843236 | 6.447811984 | 5.853372531 |
| AA147043 | 5 | 3D adult vs. fetal | 5.26712976 | 4.856265473 | 8.499381882 | 5.561464269 | 5.30607401 | 5.35955551 |
| AA035384 | 5 | 3D adult vs. fetal | 6.401728539 | 4.12666021 | 7.578771481 | 6.904866564 | 7.128981272 | 5.672655498 |
| R60150 | 5 | 3D adult vs. fetal | 4.215197474 | 4.38442636 | 4.860194724 | 4.973592031 | 5.301613877 | 4.146462455 |
| N64051 | 5 | 3D adult vs. fetal | 5.426681636 | 4.928078432 | 5.608870527 | 5.622962025 | 5.371210998 | 4.561721051 |
| AA405748 | 5 | 3D adult vs. fetal | 5.368605864 | 3.19002151 | 4.353339926 | 4.106442793 | 5.477050315 | 4.525369392 |
| AA461110 | 5 | 3D adult vs. fetal | 3.575328316 | 2.604624915 | 4.130714878 | 3.915938843 | 4.131312686 | 3.482794563 |
| AA845167 | 5 | 3D adult vs. fetal | 3.78578459 | 3.609211434 | 4.618092536 | 4.244750176 | 4.779380405 | 3.825719327 |
| AA443118 | 5 | 3D adult vs. fetal | 4.314381512 | 6.09876062 | 6.276759492 | 5.303887624 | 5.568448187 | 5.169715811 |
| N92319 | 3 | 3D adult vs. fetal | 4.572213322 | 3.033513374 | 3.354505947 | 3.067302105 | 3.243795499 | 6.319683552 |
| AA187148 | 5 | 3D adult vs. fetal | 5.511820527 | 5.984833947 | 9.561458451 | 7.538771775 | 7.330918349 | 4.996228711 |
| AA253413 | 3 | 3D adult vs. fetal | 5.753953945 | 4.708057923 | 5.13409191 | 8.221909098 | 6.216223439 | 4.722726065 |
| AA045701 | 4 | 3D adult vs. fetal | 7.244740869 | 7.004200569 | 9.55974171 | 8.437552392 | 9.263082719 | 6.552005827 |
| AA164562 | 4 | 3D adult vs. fetal | 7.627425507 | 5.216496788 | 8.699986728 | 7.923881699 | 9.508233607 | 6.844029007 |
| AA486357 | 4 | 3D adult vs. fetal | 6.41575883 | 6.975113261 | 10.40229719 | 7.96745072 | 8.41742583 | 6.451127762 |
| AA180742 | 4 | 3D adult vs. fetal | 5.52121367 | 6.234552553 | 8.103930847 | 6.983167569 | 6.946238241 | 6.26480073 |
| AA454743 | 4 | 3D adult vs. fetal | 7.511488312 | 9.369158012 | 11.34696747 | 9.562551962 | 9.456937824 | 7.36978659 |
| AA437226 | 4 | 3D adult vs. fetal | 7.112744497 | 8.791653264 | 10.80511143 | 8.266289799 | 8.507470188 | 7.034847567 |
| AA458849 | 3 | 3D adult vs. fetal | 5.469328544 | 3.66373147 | 4.903306694 | 4.827076947 | 5.660206542 | 4.514629804 |
| AA604891 | 3 | 3D adult vs. fetal | 5.258178068 | 8.251213126 | 6.90322093 | 6.369912634 | 5.676523521 | 7.288981598 |
| AA609655 | 18 | 3D adult vs. fetal | 21.33429032 | 19.08081783 | 30.01089002 | 23.9982204 | 25.31681248 | 20.18144049 |
| AA599158 | 6 | 3D adult vs. fetal | 5.73905777 | 4.765585417 | 8.346245119 | 6.423634441 | 6.61080494 | 5.063695516 |
| AA052932 | 2 | 3D adult vs. fetal | 4.160294487 | 2.183896002 | 2.762899749 | 2.096861401 | 2.192248117 | 5.413063915 |
| AA769328 | 2 | 3D adult vs. fetal | 3.49664111 | 3.795346147 | 3.543244396 | 3.202155462 | 1.664435653 | 4.680897198 |
| AA129537 | 6 | 3D adult vs. fetal | 5.490099148 | 4.033511964 | 6.753224678 | 5.270505052 | 6.104594281 | 4.571138177 |
| AA486209 | 2 | 3D adult vs. fetal | 2.951127838 | 2.622458107 | 2.396628552 | 1.876778497 | 2.154038566 | 4.999645899 |
| H39016 | 2 | 3D adult vs. fetal | 2.815824978 | 1.826966689 | 1.671944771 | 1.253970839 | 1.643279353 | 4.632563322 |
| AA464217 | 2 | 3D adult vs. fetal | 2.270265016 | 2.131631711 | 1.520659008 | 1.064618518 | 1.329400245 | 4.21418601 |
| T95053 | 2 | 3D adult vs. fetal | 2.688707692 | 5.33382498 | 1.794683232 | 1.531765242 | 1.715814755 | 5.3630072 |
| AA454640 | 2 | 3D adult vs. fetal | 2.102886248 | 1.537570373 | 1.346779699 | 1.127849141 | 1.214174754 | 4.183264486 |
| AA448400 | 2 | 3D adult vs. fetal | 3.306242137 | 2.235006202 | 1.747624063 | 0.631226114 | 1.568082834 | 8.474436694 |
| H13691 | 19 | 3D adult vs. fetal | 54.00052945 | 38.06027395 | 36.30704958 | 53.01474929 | 49.86794621 | 50.27373427 |
| AA132086 | 2 | 3D adult vs. fetal | 3.337097743 | 3.257550359 | 2.293125381 | 1.567514408 | 1.604751828 | 5.862999872 |
| AA488073 | 2 | 3D adult vs. fetal | 4.351033023 | 4.04194944 | 1.452692862 | 3.511334664 | 4.118886207 | 5.708773871 |
| N40945 | 2 | 3D adult vs. fetal | 3.447096842 | 2.154588097 | 2.501683281 | 2.52085183 | 2.297385171 | 4.762374759 |
| R55705 | 2 | 3D adult vs. fetal | 2.909308613 | 1.851341621 | 1.432688968 | 1.212749384 | 1.638105054 | 4.556458468 |
| H50114 | 2 | 3D adult vs. fetal | 3.301736569 | 1.908387922 | 1.705840938 | 1.407414465 | 1.781048668 | 4.589212608 |
| AA452841 | 2 | 3D adult vs. fetal | 3.981943611 | 1.231373773 | 1.0248198 | 0.963983432 | 1.620834153 | 4.505595396 |
| W73790 | 2 | 3D adult vs. fetal | 3.914888537 | 1.713927735 | 1.679258731 | 1.608715372 | 2.253084809 | 4.790266687 |
| N30302 | 17 | 3D adult vs. fetal | 16.236426 | 14.33825503 | 20.17733457 | 16.72882972 | 18.60385984 | 15.82011639 |
| AA291556 | 2 | 3D adult vs. fetal | 2.858389118 | 6.164431067 | 2.312941111 | 1.624968319 | 1.959001687 | 5.460005926 |
| AA598510 | 2 | 3D adult vs. fetal | 2.698323673 | 2.653383075 | 1.709197068 | 1.448745687 | 1.852274765 | 5.166038663 |
| AA453787 | 2 | 3D adult vs. fetal | 2.702589425 | 1.620596967 | 1.717336506 | 1.499079329 | 1.522616002 | 4.378038549 |
| H05655 | 2 | 3D adult vs. fetal | 3.107515398 | 2.521892845 | 1.311805573 | 1.314199679 | 1.633779252 | 6.087070828 |
| AA419177 | 18 | 3D adult vs. fetal | 23.89742962 | 15.45485589 | 25.88217585 | 24.47790114 | 28.20825412 | 20.47309058 |
| AA458807 | 18 | 3D adult vs. fetal | 22.02916221 | 16.65443357 | 25.45874485 | 22.43485375 | 25.67182927 | 18.88009347 |
| AA293218 | 18 | 3D adult vs. fetal | 19.79668624 | 15.40443015 | 27.23128953 | 22.13209523 | 21.07504567 | 18.14351405 |
| W44860 | 17 | 3D adult vs. fetal | 12.7340951 | 10.689677 | 18.07307043 | 13.89427187 | 15.92917087 | 12.73205002 |
| AA629862 | 17 | 3D adult vs. fetal | 14.59679193 | 11.53376895 | 17.21229223 | 15.59989608 | 16.64541758 | 14.01615062 |
| AA447674 | 2 | 3D adult vs. fetal | 2.534264127 | 2.150154393 | 1.427789252 | 1.000328271 | 1.423442229 | 4.137781882 |
| T52484 | 2 | 3D adult vs. fetal | 3.236398407 | 3.026511181 | 1.838920999 | 1.617002106 | 1.759710423 | 4.94920807 |
| AA496810 | 2 | 3D adult vs. fetal | 2.803422463 | 1.595989735 | 1.728921373 | 1.455020012 | 1.72732132 | 4.877833042 |
| AA486233 | 2 | 3D adult vs. fetal | 1.105294528 | 3.612629566 | 2.016514163 | 1.407057353 | 1.146012765 | 4.325105426 |
| AA079775 | 2 | 3D adult vs. fetal | 3.60412957 | 2.70567137 | 3.009055265 | 2.126182568 | 2.517322618 | 5.903186938 |
| W73889 | 2 | 3D adult vs. fetal | 3.174704174 | 4.228312305 | 3.583500944 | 3.632704149 | 3.233152734 | 6.073296967 |
| R50337 | 2 | 3D adult vs. fetal | 2.252389181 | 1.301117771 | 1.696068083 | 1.246881305 | 1.59150746 | 3.84378734 |
| R55046 | 2 | 3D adult vs. fetal | 4.804068001 | 3.914420759 | 4.479334588 | 5.310431247 | 5.155223566 | 4.292901695 |
| R46821 | 2 | 3D adult vs. fetal | 4.481781503 | 3.60517107 | 3.194315863 | 3.736730328 | 4.059831528 | 3.62674894 |
| R87763 | 2 | 3D adult vs. fetal | 4.349371289 | 1.738958085 | 2.016576353 | 1.564246255 | 2.167157105 | 4.929744779 |
| H89583 | 2 | 3D adult vs. fetal | 3.472010108 | 3.168123065 | 3.070758833 | 1.876335035 | 2.529708287 | 5.529732134 |
| R56040 | 2 | 3D adult vs. fetal | 2.875034039 | 12.02198937 | 3.636042926 | 3.134295566 | 2.862500876 | 5.657443535 |
| AA922705 | 2 | 3D adult vs. fetal | 2.467900912 | 5.753848696 | 2.364224943 | 1.826235564 | 1.990935084 | 5.56199203 |
| AA487571 | 2 | 3D adult vs. fetal | 2.392095741 | 2.030046874 | 1.8982167 | 1.167380172 | 1.445241279 | 3.891969777 |
| AA402440 | 2 | 3D adult vs. fetal | 3.04018443 | 4.537574439 | 2.816667278 | 2.313436127 | 2.567871927 | 6.461198799 |
| H29521 | 2 | 3D adult vs. fetal | 3.458079561 | 2.089088468 | 1.722877258 | 1.456445529 | 1.929668192 | 5.361928045 |
| AA490911 | 15 | 3D adult vs. fetal | 42.67030392 | 26.52859985 | 36.28250422 | 40.39466602 | 41.60811969 | 34.97057694 |
| AA486082 | 13 | 3D adult vs. fetal | 10.41727609 | 8.937884327 | 15.05040142 | 10.82056372 | 11.95282018 | 8.842288293 |
| AA678065 | 17 | 3D adult vs. fetal | 14.12093138 | 11.54070062 | 20.86168208 | 18.075708 | 17.83849384 | 13.73636722 |
| R43509 | 16 | 3D adult vs. fetal | 11.02563598 | 9.509595949 | 16.74133733 | 14.13918278 | 13.48520494 | 10.63815358 |
| N57653 | 16 | 3D adult vs. fetal | 10.4123206 | 10.44346779 | 14.67805884 | 11.74181676 | 11.74500654 | 11.69858601 |
| AA670955 | 16 | 3D adult vs. fetal | 9.121610176 | 8.547640672 | 16.37945858 | 12.25345162 | 12.21640163 | 9.258251347 |
| R14692 | 2 | 3D adult vs. fetal | 0.757198559 | 0.588063899 | 0.752811699 | 0.852655502 | 1.447179728 | 0.869563322 |
| AA488979 | 15 | 3D adult vs. fetal | 33.66640515 | 23.40785359 | 36.22684956 | 35.83780071 | 36.1199539 | 29.2581877 |
| AA443630 | 2 | 3D adult vs. fetal | 3.179366912 | 1.141747364 | 1.766810452 | 1.118906727 | 1.631839103 | 4.49413229 |
| AA027840 | 14 | 3D adult vs. fetal | 30.77584038 | 23.58752538 | 32.62815407 | 33.05944103 | 33.60205655 | 25.63272733 |
| AA458830 | 14 | 3D adult vs. fetal | 25.58379208 | 26.39848683 | 28.15722788 | 25.87408416 | 27.40987838 | 24.06147297 |
| AA459015 | 13 | 3D adult vs. fetal | 10.03176884 | 8.709006371 | 16.03563015 | 13.10174715 | 14.03043114 | 9.884872553 |
| AA074446 | 13 | 3D adult vs. fetal | 11.85807943 | 9.620046415 | 13.82726113 | 12.87569434 | 14.04256345 | 11.73842466 |
| AA027042 | 13 | 3D adult vs. fetal | 12.73491005 | 9.459896144 | 16.51983878 | 13.49994947 | 14.93224524 | 12.43468402 |
| AA629923 | 13 | 3D adult vs. fetal | 11.09427639 | 6.096835847 | 11.63735402 | 10.20004947 | 10.48961254 | 9.430135362 |
| AA460830 | 15 | 3D adult vs. fetal | 43.46763026 | 25.89585549 | 36.23321738 | 37.95742356 | 39.87905887 | 37.77709716 |
| AA454218 | 0 | 3D adult vs. fetal | 4.470293684 | 3.093239424 | 4.937529756 | 4.688122666 | 5.072419486 | 3.72227458 |
| AA046523 | 6 | 3D adult vs. fetal | 6.155805263 | 4.388837355 | 6.934532229 | 5.949254753 | 6.883321967 | 5.158132106 |
| R51346 | 0 | 3D adult vs. fetal | 3.230511245 | 3.062418161 | 3.787520043 | 3.783285032 | 3.826003563 | 2.512202334 |
| AA029964 | 0 | 3D adult vs. fetal | 3.855847135 | 4.014123191 | 5.758888572 | 4.613362037 | 4.007280855 | 3.687657081 |
| AA489219 | 0 | 3D adult vs. fetal | 3.274998157 | 2.361717684 | 3.918052974 | 3.243068103 | 3.469099389 | 3.176498463 |
| AA043133 | 0 | 3D adult vs. fetal | 3.039842745 | 3.471121389 | 5.203529353 | 4.221352079 | 4.259092185 | 3.419382811 |
| AA812973 | 17 | 3D adult vs. fetal | 15.77248197 | 10.74549128 | 18.85394679 | 18.86663157 | 16.77333141 | 12.70379222 |
| AA453471 | 0 | 3D adult vs. fetal | 3.957168294 | 3.038926799 | 2.87937811 | 4.188692511 | 4.453834895 | 3.457945931 |
| AA284693 | 17 | 3D adult vs. fetal | 14.4127364 | 9.497839898 | 13.96717518 | 12.75585136 | 13.63554527 | 14.18625183 |
| N90281 | 0 | 3D adult vs. fetal | 2.889108898 | 3.065073021 | 3.825043424 | 3.772722446 | 3.897897917 | 2.916608408 |
| AA629542 | 0 | 3D adult vs. fetal | 3.339124505 | 3.20278143 | 4.020290022 | 4.367848138 | 4.203343071 | 3.045430554 |
| AA679345 | 0 | 3D adult vs. fetal | 4.041914565 | 3.40189177 | 5.334580535 | 4.767347184 | 5.052052168 | 3.629044952 |
| H37774 | 2 | 3D adult vs. fetal | 2.582136051 | 5.751409544 | 0.826130431 | 0.749198653 | 0.950396052 | 4.924705249 |
| T97181 | 2 | 3D adult vs. fetal | 2.848144946 | 1.762304259 | 1.780931817 | 1.662977237 | 1.930489483 | 4.429079742 |
| AA454879 | 2 | 3D adult vs. fetal | 2.588814818 | 1.895822968 | 0.788622997 | 0.683939522 | 0.868426864 | 4.58445785 |
| AA147640 | 0 | 3D adult vs. fetal | 3.111174182 | 3.689139309 | 3.781524296 | 4.096860799 | 3.959011774 | 2.732271995 |
| AA767429 | 9 | 3D adult vs. fetal | 7.054093591 | 5.363580152 | 7.988105567 | 6.839604508 | 7.370692262 | 7.981701609 |
| AA490991 | 9 | 3D adult vs. fetal | 10.98757317 | 7.790729538 | 12.35710247 | 9.885681916 | 11.00435577 | 8.898305631 |
| AA422058 | 17 | 3D adult vs. fetal | 14.71408525 | 14.84685565 | 24.49176062 | 19.94632048 | 19.29291385 | 14.68049409 |
| N66208 | 9 | 3D adult vs. fetal | 8.123797741 | 6.177962556 | 10.33188119 | 7.894725917 | 8.580898297 | 6.881934203 |
| AA630776 | 9 | 3D adult vs. fetal | 9.138119273 | 5.83233298 | 11.41953939 | 9.003209098 | 9.590093969 | 8.070647196 |
| AA827287 | 9 | 3D adult vs. fetal | 8.482649348 | 7.482270307 | 8.891746969 | 9.19984268 | 9.976725792 | 8.422494842 |
| AA488084 | 9 | 3D adult vs. fetal | 7.649134152 | 14.97955716 | 13.41766713 | 10.42093881 | 9.071703388 | 8.874743158 |
| R89715 | 13 | 3D adult vs. fetal | 9.750099751 | 17.8705502 | 18.03616416 | 13.82091334 | 12.99735343 | 14.90421282 |
| AA490501 | 9 | 3D adult vs. fetal | 7.944415444 | 7.85975819 | 11.06033198 | 9.284097489 | 8.992468434 | 7.63431741 |
| N32199 | 9 | 3D adult vs. fetal | 9.67828656 | 7.893329723 | 9.916311099 | 9.837236279 | 11.23987563 | 9.150031823 |
| AA434404 | 9 | 3D adult vs. fetal | 6.86730459 | 8.426674472 | 9.577263322 | 8.037236674 | 8.096867575 | 6.953180054 |
| N93688 | 8 | 3D adult vs. fetal | 5.038274076 | 10.94187624 | 9.036823839 | 7.358456237 | 7.155250657 | 6.336977302 |
| AA292676 | 7 | 3D adult vs. fetal | 11.09420423 | 9.867844396 | 9.548860422 | 11.52855409 | 12.53274971 | 9.824831453 |
| AA464417 | 7 | 3D adult vs. fetal | 10.40978662 | 9.6398749 | 11.95762629 | 12.77651551 | 12.8767479 | 10.07601153 |
| AA442092 | 7 | 3D adult vs. fetal | 9.965987122 | 7.370289148 | 8.883701613 | 9.441691799 | 9.667688043 | 8.214697052 |
| AA442092 | 7 | 3D adult vs. fetal | 9.965987122 | 7.370289148 | 8.883701613 | 9.441691799 | 9.667688043 | 8.214697052 |
| AA026644 | 7 | 3D adult vs. fetal | 7.769602383 | 6.638214592 | 5.137119018 | 3.572083072 | 4.086915401 | 15.10517784 |
| AA481464 | 9 | 3D adult vs. fetal | 7.356616049 | 9.394727631 | 11.96871523 | 9.624488789 | 9.697819022 | 7.196159079 |
| T68859 | 12 | 3D adult vs. fetal | 7.102239287 | 6.063011273 | 10.14342977 | 8.316121437 | 8.90719198 | 7.165656319 |
| AA699560 | 13 | 3D adult vs. fetal | 9.978421471 | 9.37295778 | 14.75863286 | 16.42689227 | 14.26471651 | 12.73562928 |
| AA705069 | 13 | 3D adult vs. fetal | 8.512749427 | 10.51595378 | 13.19983017 | 9.999000301 | 10.13397175 | 9.694546633 |
| AA457739 | 13 | 3D adult vs. fetal | 13.76296688 | 10.27635708 | 15.4062123 | 12.76652957 | 14.0993498 | 10.82804945 |
| H99843 | 13 | 3D adult vs. fetal | 11.42545366 | 9.860489604 | 11.08358091 | 10.09631162 | 9.902204667 | 10.73353077 |
| AA399410 | 13 | 3D adult vs. fetal | 11.49268913 | 9.868304848 | 16.50988414 | 14.49672712 | 13.23658995 | 10.91245055 |
| AA443039 | 9 | 3D adult vs. fetal | 8.661615681 | 5.473553572 | 10.7592073 | 8.268347579 | 9.771073691 | 7.541136242 |
| AA164440 | 11 | 3D adult vs. fetal | 17.82989233 | 11.87877628 | 20.35395821 | 18.48267711 | 19.46815644 | 14.70024382 |
| AA446453 | 11 | 3D adult vs. fetal | 16.55396138 | 13.81116994 | 16.50294651 | 19.54048563 | 21.22775124 | 16.02767268 |
| AA280692 | 11 | 3D adult vs. fetal | 17.40750225 | 16.18669704 | 19.04935817 | 19.71347085 | 20.1673057 | 16.35083848 |
| AA031514 | 11 | 3D adult vs. fetal | 20.58635757 | 17.50162009 | 21.03844179 | 22.39941193 | 21.41470955 | 18.33395551 |
| R33154 | 11 | 3D adult vs. fetal | 15.80929053 | 13.23696939 | 13.37191076 | 14.52813826 | 14.95312607 | 16.05415087 |
| AA487452 | 11 | 3D adult vs. fetal | 13.71327193 | 19.41866534 | 12.61831663 | 12.077349 | 12.74498437 | 18.11228999 |
| AA400329 | 9 | 3D adult vs. fetal | 9.471410325 | 8.139099224 | 9.945496246 | 10.13837609 | 11.074429 | 7.250594994 |
| AA454668 | 13 | 3D adult vs. fetal | 9.047350014 | 7.5254115 | 12.60652925 | 10.81932261 | 10.34313891 | 8.424799177 |
| AA486393 | 23 | 3D adult vs. failure | 0.682030067 | 6.724440565 | 0.800620868 | 0.562299276 | 0.603381368 | 1.639697615 |
| R62541 | 23 | 3D adult vs. failure | 0.402099116 | 2.047232943 | 0.476534818 | 0.419746076 | 0.364790601 | 0.848514165 |
| AA171613 | 23 | 3D adult vs. failure | 0.833916304 | 6.428013019 | 1.049489533 | 0.831618144 | 1.061543998 | 2.831110499 |
| AA235706 | 0 | 3D adult vs. failure | 103.2862145 | 45.18459033 | 36.30704958 | 66.22096461 | 76.19336098 | 53.68696068 |
| AA668527 | 23 | 3D adult vs. failure | 1.360133945 | 6.805032154 | 1.071215078 | 0.97529683 | 1.06310668 | 3.121521767 |
| T64144 | 23 | 3D adult vs. failure | 0.476915725 | 7.417782102 | 0.746262622 | 0.455935776 | 0.588907616 | 1.573719963 |
| R14080 | 23 | 3D adult vs. failure | 0.442812641 | 7.906110384 | 0.627950807 | 0.498614946 | 0.372466179 | 1.218619609 |
| AA609599 | 23 | 3D adult vs. failure | 0.359352461 | 7.143369152 | 0.569305177 | 0.366281857 | 0.341488403 | 1.345297086 |
| AA489201 | 23 | 3D adult vs. failure | 0.746247673 | 7.761267038 | 0.569230512 | 0.568039398 | 0.51445757 | 1.823393758 |
| R08876 | 23 | 3D adult vs. failure | 0.881300108 | 6.204438487 | 1.346594192 | 1.127374945 | 1.303511748 | 1.365140688 |
| H46425 | 23 | 3D adult vs. failure | 0.446119129 | 7.549550263 | 0.42423628 | 0.384125705 | 0.304256579 | 1.948954368 |
| R56149 | 20 | 3D adult vs. failure | 3.946739485 | 2.221889535 | 2.687396748 | 2.29349791 | 2.994436137 | 6.00466275 |
| AA454619 | 20 | 3D adult vs. failure | 4.670053967 | 2.36899368 | 3.285602699 | 2.811568095 | 3.530544823 | 6.168078813 |
| H15445 | 20 | 3D adult vs. failure | 3.638147066 | 2.706447087 | 1.458885277 | 1.457265129 | 2.021283714 | 6.315381377 |
| AA705225 | 23 | 3D adult vs. failure | 1.386836786 | 6.429576897 | 1.83711377 | 1.34483513 | 1.150500104 | 2.597429853 |
| AA191488 | 2 | 3D adult vs. failure | 2.174224052 | 45.10144344 | 2.069008838 | 1.631870516 | 1.77653582 | 16.32613468 |
| N64862 | 14 | 3D adult vs. failure | 0.650596095 | 12.10838017 | 1.065306504 | 0.665682947 | 0.614517248 | 3.167265877 |
| R45413 | 7 | 3D adult vs. failure | 0.716578413 | 26.61952355 | 1.065046051 | 0.917779985 | 0.773891766 | 6.012284228 |
| R77293 | 7 | 3D adult vs. failure | 4.173270014 | 18.87787869 | 6.098081459 | 6.158463331 | 5.192489399 | 6.808820493 |
| AA436187 | 6 | 3D adult vs. failure | 7.73084558 | 34.64438609 | 9.740301386 | 7.582520052 | 8.233604749 | 18.4559413 |
| AA676470 | 5 | 3D adult vs. failure | 23.09461759 | 12.79749882 | 19.67261192 | 18.83195374 | 25.31283192 | 19.5015243 |
| AA443634 | 23 | 3D adult vs. failure | 0.483674724 | 6.507766668 | 0.834546801 | 0.602237179 | 0.563061629 | 1.705323839 |
| AA664180 | 4 | 3D adult vs. failure | 77.28842805 | 43.66418943 | 36.30704958 | 68.26659959 | 73.01397471 | 54.50243711 |
| W58658 | 20 | 3D adult vs. failure | 3.738957043 | 1.157647486 | 1.625298607 | 0.567045634 | 1.362263758 | 5.290498458 |
| H54023 | 2 | 3D adult vs. failure | 2.518425666 | 76.64764128 | 1.455767277 | 1.89558391 | 2.244043815 | 23.68020326 |
| H73724 | 11 | 3D adult vs. failure | 0.794565272 | 15.90487626 | 1.124742532 | 0.660242857 | 0.688814595 | 4.296719254 |
| T70031 | 2 | 3D adult vs. failure | 2.006155548 | 48.92472369 | 2.789740103 | 2.01477414 | 1.746671803 | 15.29766287 |
| AA481758 | 0 | 3D adult vs. failure | 123.6211644 | 61.20693911 | 36.30704958 | 92.35920856 | 102.0498037 | 54.85301063 |
| AA521431 | 0 | 3D adult vs. failure | 125.2413904 | 56.77999766 | 36.30704958 | 93.6827224 | 100.8362156 | 54.85301063 |
| AA446103 | 23 | 3D adult vs. failure | 0.338624303 | 7.652704012 | 0.571045963 | 0.349989394 | 0.406883851 | 1.434663101 |
| N92646 | 5 | 3D adult vs. failure | 30.42865911 | 14.89709888 | 29.90172172 | 26.59339088 | 28.56672605 | 26.04183434 |
| AA453789 | 18 | 3D adult vs. failure | 2.044150452 | 8.495958017 | 2.328026154 | 2.572167528 | 2.212159729 | 3.457082607 |
| AA425299 | 18 | 3D adult vs. failure | 7.887770078 | 3.918963058 | 10.38387994 | 7.660089272 | 8.794304531 | 7.329938113 |
| AA868929 | 18 | 3D adult vs. failure | 1.98091121 | 9.937368538 | 2.013089465 | 1.471073914 | 1.420828724 | 5.418655459 |
| R60019 | 18 | 3D adult vs. failure | 2.116044289 | 8.525464296 | 1.047037018 | 0.928042539 | 1.082727505 | 5.235772895 |
| AA857343 | 18 | 3D adult vs. failure | 1.650037418 | 6.297808737 | 2.426871459 | 1.704623126 | 1.741966813 | 3.845429741 |
| AA481438 | 18 | 3D adult vs. failure | 2.573933498 | 8.391310909 | 2.445625176 | 1.893217317 | 1.76341218 | 6.043734557 |
| AA399674 | 18 | 3D adult vs. failure | 2.130783417 | 8.164685436 | 3.388970859 | 2.354039954 | 2.279993542 | 4.872785488 |
| T98887 | 18 | 3D adult vs. failure | 1.100740148 | 8.756420509 | 0.936100822 | 1.100317922 | 0.803584044 | 3.484794007 |
| AA678404 | 18 | 3D adult vs. failure | 1.601598727 | 7.015197466 | 1.932547665 | 1.820935977 | 1.80852363 | 3.313612045 |
| H15747 | 20 | 3D adult vs. failure | 3.703368787 | 1.841236561 | 1.31983711 | 0.468266355 | 1.579189807 | 6.384201738 |
| H16958 | 16 | 3D adult vs. failure | 4.911652574 | 2.505777016 | 6.153238607 | 3.258278988 | 9.421877751 | 2.976025283 |
| AA936783 | 14 | 3D adult vs. failure | 1.342031028 | 13.57092172 | 1.952974192 | 1.334954568 | 1.22240126 | 1.341547085 |
| AA884709 | 18 | 3D adult vs. failure | 2.262402026 | 7.467527538 | 3.158379807 | 2.652355075 | 2.663007264 | 4.51372877 |
| H24688 | 18 | 3D adult vs. failure | 1.395480263 | 7.059371875 | 2.232669864 | 1.921932565 | 1.963742023 | 4.014560107 |
| AA884403 | 18 | 3D adult vs. failure | 2.032526808 | 8.508216003 | 2.408869338 | 2.267218044 | 2.281983003 | 4.451591143 |
| AA404619 | 19 | 3D adult vs. failure | 0.721996555 | 11.04039228 | 0.849034927 | 0.475083665 | 0.645471218 | 2.92914746 |
| AA598611 | 19 | 3D adult vs. failure | 0.473381741 | 9.658226818 | 0.485642718 | 0.382776293 | 0.327497562 | 3.321739 |
| H72875 | 19 | 3D adult vs. failure | 1.047528607 | 9.965593016 | 1.7514717648 | 0.96145731 | 0.718146053 | 3.174877726 |
| H63361 | 19 | 3D adult vs. failure | 0.444705701 | 8.608586298 | 0.460617918 | 0.347764767 | 0.327526489 | 2.835780951 |
| R39221 | 19 | 3D adult vs. failure | 0.063457623 | 8.80369405 | 0.916404688 | 0.588441567 | 0.583832961 | 2.21717036 |
| R02348 | 19 | 3D adult vs. failure | 0.380957115 | 8.723284539 | 0.689843744 | 0.430680409 | 0.314907846 | 1.628641297 |
| R51835 | 19 | 3D adult vs. failure | 0.319821038 | 3.205091988 | 0.499133148 | 0.341800718 | 0.398772973 | 0.702868877 |
| R33031 | 19 | 3D adult vs. failure | 0.309199103 | 9.871901353 | 0.870719013 | 0.498982302 | 0.446815901 | 1.737947999 |
| AA412053 | 18 | 3D adult vs. failure | 2.173462159 | 8.658749377 | 1.560525939 | 2.335301865 | 1.60001113 | 3.312435004 |
| AA001897 | 11 | 3D adult vs. failure | 0.707399619 | 18.56199185 | 1.276833043 | 0.839394093 | 0.698445289 | 3.44857855 |
| W81191 | 18 | 3D adult vs. failure | 2.035130827 | 9.564497621 | 1.470028438 | 1.278460058 | 1.466681545 | 5.442558928 |
| AA430552 | 16 | 3D adult vs. failure | 6.704743363 | 3.912744879 | 7.308427625 | 6.182487835 | 6.715639324 | 5.892347254 |
| AA594130 | 20 | 3D adult vs. failure | 4.197684546 | 1.950948544 | 3.399747789 | 2.792799271 | 3.308148608 | 6.573561928 |
| N92864 | 20 | 3D adult vs. failure | 3.964889092 | 1.12030555 | 1.255047434 | 1.028875207 | 1.597688392 | 6.129597747 |
| AA457123 | 20 | 3D adult vs. failure | 3.825435082 | 1.372454684 | 1.737581719 | 1.805411888 | 2.242579776 | 5.776097858 |
| R43320 | 19 | 3D adult vs. failure | 0.534524185 | 10.13392358 | 0.307540341 | 0.334762409 | 0.297154033 | 3.300796986 |
| AA670430 | 20 | 3D adult vs. failure | 6.033965543 | 2.411089781 | 6.490413776 | 5.227507565 | 6.00488788 | 4.958892131 |
| H85068 | 11 | 3D adult vs. failure | 0.932240825 | 19.08305372 | 1.062851862 | 1.323524085 | 0.843208322 | 3.42676519 |
| AA458785 | 11 | 3D adult vs. failure | 1.024544481 | 14.76220711 | 1.673515465 | 1.016303139 | 1.117923958 | 3.660946499 |
| AA485871 | 11 | 3D adult vs. failure | 1.240425485 | 16.32124036 | 1.819664978 | 1.051475658 | 1.243832378 | 4.885742561 |
| T39411 | 14 | 3D adult vs. failure | 0.683416906 | 14.1280482 | 0.827762784 | 0.665895165 | 0.776309726 | 3.661139864 |
| R00855 | 20 | 3D adult vs. failure | 4.116868708 | 2.545089683 | 2.768201659 | 2.658761885 | 2.788015121 | 6.090967819 |
| H98666 | 16 | 3D adult vs. failure | 7.496261053 | 3.423039388 | 3.917399183 | 3.208047795 | 4.021338011 | 7.436138134 |
| H72028 | 14 | 3D adult vs. failure | 1.122387248 | 10.87725529 | 1.107269289 | 0.894651185 | 0.693097551 | 5.481280912 |
| AA679177 | 14 | 3D adult vs. failure | 0.813601986 | 11.3980019 | 0.884729818 | 0.738756843 | 0.624486657 | 3.353148409 |
| N21578 | 14 | 3D adult vs. failure | 0.493867961 | 12.22018711 | 0.630068988 | 0.332239214 | 0.452874405 | 3.48979683 |
| AA007419 | 14 | 3D adult vs. failure | 0.640548282 | 10.87290796 | 0.573707373 | 0.426598512 | 0.390973266 | 5.000691355 |
| T49657 | 14 | 3D adult vs. failure | 1.478438588 | 12.2454622 | 1.66172702 | 1.085853458 | 1.158150176 | 4.728340076 |
| N38959 | 14 | 3D adult vs. failure | 0.96668518 | 12.21521754 | 1.222240919 | 0.390241965 | 1.036587317 | 3.877992922 |
| R51912 | 14 | 3D adult vs. failure | 0.931685691 | 13.32666832 | 0.554065456 | 0.584470901 | 0.545379233 | 4.794685202 |
| H90415 | 14 | 3D adult vs. failure | 1.027933738 | 14.40938868 | 1.543560847 | 1.015015198 | 0.913466897 | 3.697172386 |
| H41489 | 1 | 2D/3D adult vs. fetal | 4.027389786 | 2.097422239 | 1.726349793 | 1.413362414 | 2.223616983 | 7.753646206 |
| H16456 | 1 | 2D/3D adult vs. fetal | 4.015386257 | 7.010456083 | 2.974647228 | 2.512493381 | 3.106940998 | 8.704678862 |
| W45415 | 1 | 2D/3D adult vs. fetal | 5.612363827 | 1.065216881 | 2.691703268 | 2.027056421 | 2.568973782 | 6.615995267 |
| AA447751 | 1 | 2D/3D adult vs. fetal | 3.707303465 | 4.780241073 | 2.223590687 | 2.437972778 | 2.631458156 | 7.623606101 |
| AA487486 | 1 | 2D/3D adult vs. fetal | 4.21336596 | 4.045765801 | 3.015283294 | 2.286202019 | 2.768948605 | 8.048478397 |
| R56604 | 1 | 2D/3D adult vs. fetal | 4.165186307 | 3.846779908 | 2.973815371 | 2.369929936 | 3.1509812 | 7.458099126 |
| T65772 | 1 | 2D/3D adult vs. fetal | 4.487863913 | 2.538286605 | 2.643015763 | 2.462140712 | 3.0062686448 | 0.741762884 |
| H15085 | 1 | 2D/3D adult vs. fetal | 4.532070688 | 2.552606255 | 1.874728576 | 1.797928653 | 2.249323538 | 7.10815384 |
| R61295 | 1 | 2D/3D adult vs. fetal | 5.7522625 | 3.64542013 | 2.945120833 | 2.564363772 | 2.826103944 | 7.68061273 |
| T61256 | 1 | 2D/3D adult vs. fetal | 5.260364 | 1.641968589 | 1.819566112 | 1.625856706 | 2.432049565 | 6.879367501 |
| AA405731 | 0 | 2D/3D adult vs. fetal | 2.090454022 | 34.42033666 | 1.664515998 | 1.304288382 | 1.293105338 | 10.6339838 |
| T71879 | 1 | 2D/3D adult vs. fetal | 4.193732889 | 4.210242665 | 1.985666897 | 1.946658119 | 2.195662508 | 8.143137484 |
| R59927 | 1 | 2D/3D adult vs. fetal | 4.826208077 | 4.383351971 | 1.602344146 | 1.52557434 | 2.192290627 | 7.925477266 |
| AA496780 | 0 | 2D/3D adult vs. fetal | 1.514704846 | 26.44459843 | 1.135159078 | 0.912692615 | 1.001084793 | 10.48006188 |
| AA176888 | 0 | 2D/3D adult vs. fetal | 0.883613375 | 16.90708911 | 0.969072645 | 0.759370466 | 0.781878516 | 6.758214974 |
| AA436163 | 0 | 2D/3D adult vs. fetal | 1.145286483 | 19.39007997 | 0.91032868 | 0.79908737 | 1.062812492 | 7.470804263 |
| AA428778 | 0 | 2D/3D adult vs. fetal | 2.705936237 | 23.81604244 | 1.650059764 | 1.291875843 | 1.526455491 | 10.05785097 |
| AA463225 | 0 | 2D/3D adult vs. fetal | 1.805467836 | 22.97689102 | 1.545226155 | 1.416083191 | 1.289595725 | 9.717200851 |
| AA485426 | 0 | 2D/3D adult vs. fetal | 1.389325695 | 25.67139954 | 1.27039293 | 1.069742478 | 0.998457685 | 9.858144828 |
| W47485 | 0 | 2D/3D adult vs. fetal | 1.745341956 | 27.39946025 | 1.287668441 | 1.305011386 | 1.416492719 | 11.04790889 |
| H84982 | 0 | 2D/3D adult vs. fetal | 1.533032723 | 28.30131199 | 1.047589817 | 1.549633971 | 1.23686481 | 9.828318446 |
| AA504615 | 1 | 2D/3D adult vs. fetal | 4.050257188 | 3.625356301 | 2.199703889 | 1.941384876 | 2.75118289 | 7.641495993 |
| H94487 | 0 | 2D/3D adult vs. fetal | 1.645750443 | 22.47903843 | 3.594820042 | 1.028845936 | 1.681686149 | 8.286429124 |
| AA448959 | 0 | 2D/3D adult vs. fetal | 3.124990125 | 4.592355812 | 3.899374735 | 3.173734392 | 3.451263958 | 6.793092921 |
| AA070358 | 2 | 2D/3D adult vs. fetal | 8.012667475 | 1.564102379 | 1.883538283 | 1.584996753 | 2.642393389 | 9.48000834 |
| AA453401 | 1 | 2D/3D adult vs. fetal | 5.692934592 | 1.528593629 | 1.905652383 | 1.904350696 | 2.206453207 | 6.672888766 |
| N66737 | 14 | 2D/3D adult vs. fetal | 24.90073908 | 0.393334078 | 1.095420779 | 0.556298638 | 0.923759381 | 3.442649218 |
| AA666180 | 9 | 2D/3D adult vs. fetal | 2.115740805 | 12.05725753 | 2.14570443 | 1.488045473 | 1.870792054 | 5.34373246 |
| AA857131 | 9 | 2D/3D adult vs. fetal | 0.152032673 | 0.207460704 | 0.268997471 | 0.210685319 | 0.132367162 | 0.357440795 |
| AA479102 | 9 | 2D/3D adult vs. fetal | 2.295197382 | 25.01632531 | 3.681214605 | 3.425928026 | 3.02851033 | 8.49611205 |
| AA456077 | 7 | 2D/3D adult vs. fetal | 7.731170292 | 4.509291885 | 4.761631135 | 3.922605205 | 4.875603995 | 11.60167555 |
| R87497 | 7 | 2D/3D adult vs. fetal | 7.553857203 | 4.518297667 | 3.006217917 | 1.067794468 | 4.393661357 | 12.60889243 |
| AA718910 | 7 | 2D/3D adult vs. fetal | 7.12414318 | 3.548431737 | 3.928552441 | 3.557814726 | 4.383101911 | 10.82120865 |
| AA406269 | 7 | 2D/3D adult vs. fetal | 5.887777587 | 6.493700059 | 3.376570613 | 2.359008775 | 3.447900579 | 11.90806873 |
| N74623 | 4 | 2D/3D adult vs. fetal | 0.738692411 | 0.885632206 | 0.507569185 | 0.423927496 | 0.405894222 | 1.326758097 |
| H99364 | 6 | 2D/3D adult vs. fetal | 4.654446832 | 3.967945222 | 3.173155342 | 2.53020258 | 2.826020625 | 9.199591084 |
| AA447684 | 6 | 2D/3D adult vs. fetal | 3.976408745 | 14.43816072 | 4.763738151 | 2.471882235 | 2.732135077 | 8.763139341 |
| AA282301 | 6 | 2D/3D adult vs. fetal | 5.741753046 | 2.638926534 | 3.525955597 | 3.116535142 | 3.442563042 | 8.08592064 |
| H99588 | 5 | 2D/3D adult vs. fetal | 1.455459706 | 29.42112357 | 0.93818073 | 0.705663294 | 0.91408074 | 9.587415089 |
| N53512 | 5 | 2D/3D adult vs. fetal | 1.885890833 | 27.43755749 | 1.437831064 | 1.488193294 | 1.489514434 | 11.66412529 |
| AA683321 | 5 | 2D/3D adult vs. fetal | 1.133236265 | 27.22345134 | 1.394968386 | 0.968365361 | 0.71972912 | 8.309498154 |
| AA608557 | 5 | 2D/3D adult vs. fetal | 2.577273943 | 29.04646478 | 2.807387159 | 2.029202014 | 1.932689792 | 8.478935818 |
| AA757764 | 2 | 2D/3D adult vs. fetal | 5.690914196 | 3.357895765 | 3.881101888 | 2.752079821 | 2.955536228 | 9.043391314 |
| AA406064 | 2 | 2D/3D adult vs. fetal | 6.957108219 | 3.855068049 | 3.928274978 | 3.830306615 | 5.143577569 | 9.248530935 |
| N54598 | 20 | 2D/3D adult vs. fetal | 6.411864475 | 40.45864114 | 12.32476105 | 8.740060641 | 8.361428458 | 11.67500348 |
| AA481988 | 7 | 2D/3D adult vs. fetal | 6.029716769 | 7.619281816 | 3.801345873 | 3.597022308 | 3.988124217 | 12.08244483 |
| N62394 | 3 | 2D Marker adult vs. fetal | 2.436813979 | 3.687865805 | 1.411911686 | 4.045770491 | 0.844150341 | 2.656973499 |
| N20148 | 3 | 2D Marker adult vs. fetal | 3.44995341 | 2.759942512 | 3.751806548 | 3.90090485 | 3.142378907 | 4.04494813 |
| AA496678 | 3 | 2D Marker adult vs. fetal | 7.001100843 | 4.03568563 | 5.297409583 | 3.722383923 | 5.160647823 | 10.19230959 |
| AA400973 | 3 | 2D Marker adult vs. fetal | 2.89346803 | 4.47076738 | 4.470210209 | 3.193478491 | 3.257043268 | 4.020015252 |
| AA497027 | 3 | 2D Marker adult vs. fetal | 1.802250483 | 0.578162102 | 3.922092662 | 2.407065102 | 1.286762868 | 2.157496735 |
| N64508 | 3 | 2D Marker adult vs. fetal | 2.115795984 | 3.276856168 | 1.90235047 | 5.314258483 | 2.788331395 | 3.540796101 |
| AA033564 | 3 | 2D Marker adult vs. fetal | 5.316788651 | 2.720044947 | 3.133883202 | 2.683871596 | 3.532284326 | 8.161295359 |
| AA446108 | 3 | 2D Marker adult vs. fetal | 2.078301935 | 2.211041944 | 3.792759943 | 3.324684095 | 3.375402585 | 2.756568814 |
| AA159577 | 3 | 2D Marker adult vs. fetal | 4.746090464 | 4.989070689 | 4.287813007 | 3.593768792 | 3.816425244 | 7.786959581 |
| R36958 | 3 | 2D Marker adult vs. fetal | 4.749539144 | 3.400588787 | 5.624793806 | 5.234422192 | 5.286395351 | 4.078616944 |
| AA829508 | 3 | 2D Marker adult vs. fetal | 1.920163507 | 0.7909906 | 3.923258324 | 3.457491381 | 1.13174848 | 1.710017559 |
| AA482067 | 3 | 2D Marker adult vs. fetal | 6.194625221 | 4.238881966 | 3.254611387 | 3.315340662 | 3.879401032 | 8.171254871 |
| AA669314 | 3 | 2D Marker adult vs. fetal | 6.809538669 | 4.133050912 | 5.329740351 | 3.780076886 | 4.424821889 | 8.385883026 |
| AA775241 | 3 | 2D Marker adult vs. fetal | 6.014247582 | 4.222679024 | 5.909433903 | 4.651935893 | 4.529769385 | 7.71821785 |
| R73584 | 3 | 2D Marker adult vs. fetal | 2.788220682 | 5.744755801 | 4.174172878 | 3.110031036 | 2.840523285 | 3.830886431 |
| H28984 | 5 | 2D Marker adult vs. fetal | 2.892697648 | 3.831327794 | 4.337837094 | 4.289702271 | 3.643114467 | 3.029043454 |
| R44202 | 3 | 2D Marker adult vs. fetal | 3.843410664 | 2.636358787 | 3.786734961 | 3.415590384 | 3.473443896 | 3.189323952 |
| W70051 | 4 | 2D Marker adult vs. fetal | 0.460743655 | 0.435637611 | 0.599111691 | 0.634647941 | 0.684508861 | 0.518398472 |
| AA401972 | 5 | 2D Marker adult vs. fetal | 4.694309329 | 5.237266898 | 4.803264368 | 6.3957827 | 6.187809857 | 5.273916388 |
| AA236164 | 5 | 2D Marker adult vs. fetal | 4.489190329 | 5.999132856 | 6.533218517 | 5.791985355 | 5.205993927 | 5.089326434 |
| R22412 | 5 | 2D Marker adult vs. fetal | 3.322360737 | 8.924589287 | 5.257612264 | 4.2528688 | 4.433039078 | 4.372991798 |
| AA424804 | 5 | 2D Marker adult vs. fetal | 6.72316462 | 4.945597359 | 8.844706058 | 7.100383442 | 7.690736998 | 5.712216735 |
| AA669443 | 5 | 2D Marker adult vs. fetal | 3.473141273 | 5.642074949 | 5.804136792 | 4.780513481 | 4.819190027 | 3.624913174 |
| N69689 | 2 | 2D Marker adult vs. fetal | 1.47268426 | 1.500609747 | 1.577772215 | 1.63158512 | 1.673578671 | 1.20775425 |
| H24316 | 4 | 2D Marker adult vs. fetal | 4.097759837 | 4.571809686 | 6.670594998 | 5.519559327 | 5.302346746 | 4.332423617 |
| AA074224 | 3 | 2D Marker adult vs. fetal | 2.916040437 | 42.17908573 | 3.996084321 | 3.421263628 | 2.977614141 | 15.7136877 |
| R36571 | 4 | 2D Marker adult vs. fetal | 1.22088245 | 0.697979245 | 1.246173941 | 1.106008283 | 1.434340053 | 1.173334441 |
| AA058465 | 4 | 2D Marker adult vs. fetal | 5.113683679 | 10.12779251 | 5.977620638 | 5.183053806 | 5.656909478 | 5.941479667 |
| AA633811 | 4 | 2D Marker adult vs. fetal | 5.047585747 | 3.541329987 | 5.859700035 | 4.917562173 | 6.033089317 | 4.6898963101 |
| AA457155 | 4 | 2D Marker adult vs. fetal | 4.59919155 | 2.634463051 | 5.207796008 | 3.444600254 | 3.888657977 | 4.888009989 |
| AA459104 | 4 | 2D Marker adult vs. fetal | 4.790849026 | 2.49717776 | 4.875933559 | 4.052637562 | 4.532277466 | 3.791654155 |
| R40212 | 4 | 2D Marker adult vs. fetal | 5.695606523 | 6.536605083 | 5.50014925 | 4.878309221 | 5.754823259 | 8.404118354 |
| AA086476 | 4 | 2D Marker adult vs. fetal | 3.826188364 | 2.338234619 | 4.05845178 | 3.573649092 | 3.942866534 | 3.486560771 |
| AA683310 | 5 | 2D Marker adult vs. fetal | 4.988875925 | 0.427535974 | 6.884450204 | 7.059542516 | 6.212911071 | 5.167664215 |
| AA455640 | 1 | 2D Marker adult vs. fetal | 2.29694825 | 2.246509212 | 2.490940902 | 2.65444359 | 2.528889751 | 2.024350751 |
| AA496878 | 1 | 2D Marker adult vs. fetal | 2.329206672 | 2.464809211 | 2.708923499 | 2.591526171 | 2.820757906 | 2.528269298 |
| AA085749 | 1 | 2D Marker adult vs. fetal | 1.134352576 | 1.145219423 | 0.919694908 | 1.213892433 | 1.191952306 | 1.148805945 |
| AA425755 | 1 | 2D Marker adult vs. fetal | 1.256026253 | 1.42629856 | 1.456101082 | 1.538376189 | 1.521167266 | 0.95599115 |
| N52350 | 1 | 2D Marker adult vs. fetal | 2.159542225 | 2.651354597 | 2.427419475 | 2.256040758 | 2.484151334 | 2.217863077 |
| AA630104 | 1 | 2D Marker adult vs. fetal | 1.898590687 | 1.850429013 | 2.323118997 | 2.265340283 | 2.543886171 | 1.567701571 |
| AA454854 | 1 | 2D Marker adult vs. fetal | 1.85505455 | 1.874219048 | 2.289079847 | 2.076882204 | 1.775527487 | 1.600396337 |
| W73406 | 2 | 2D Marker adult vs. fetal | 1.145861817 | 1.688694122 | 1.394720158 | 1.5150059 | 1.348266115 | 1.257230067 |
| R12802 | 1 | 2D Marker adult vs. fetal | 0.754036384 | 0.458756191 | 2.051238468 | 1.123879939 | 0.741672703 | 6.651716983 |
| AA465355 | 2 | 2D Marker adult vs. fetal | 2.427318967 | 1.234497382 | 1.382975298 | 2.820706627 | 1.758025333 | 2.561266549 |
| AA829383 | 1 | 2D Marker adult vs. fetal | 0.627874118 | 0.361600207 | 0.637447262 | 0.608221718 | 0.513908343 | 0.712979229 |
| AA629189 | 1 | 2D Marker adult vs. fetal | 3.384460323 | 3.907165525 | 3.933343191 | 4.23836901 | 3.468746044 | 3.982435808 |
| AA430512 | 0 | 2D Marker adult vs. fetal | 2.239479445 | 2.637756736 | 3.81876171 | 3.36943129 | 3.404540535 | 2.413395122 |
| AA456439 | 0 | 2D Marker adult vs. fetal | 3.838221727 | 2.779439139 | 4.396947289 | 4.325871481 | 4.445703612 | 3.414286314 |
| H27864 | 0 | 2D Marker adult vs. fetal | 4.083067399 | 2.588260193 | 3.247167731 | 3.682989494 | 4.052835461 | 3.076145132 |
| AA644657 | 0 | 2D Marker adult vs. fetal | 2.749565753 | 1.949529953 | 3.282718575 | 2.77240442 | 2.835386395 | 2.393138312 |
| R40460 | 0 | 2D Marker adult vs. fetal | 2.617741285 | 2.77044995 | 2.345768887 | 2.530348937 | 2.639132034 | 2.129569504 |
| W96058 | 1 | 2D Marker adult vs. fetal | 0.551793053 | 0.719935768 | 0.823172955 | 0.716948884 | 0.647054285 | 0.511406835 |
| T72202 | 2 | 2D Marker adult vs. fetal | 1.372133229 | 0.654777758 | 1.354496565 | 1.335828319 | 1.299148537 | 1.132910683 |
| AA598794 | 3 | 2D Marker adult vs. fetal | 0.857314271 | 0.38760124 | 4.80929041 | 4.449090919 | 3.2675415 | 0.950844105 |
| AA599178 | 3 | 2D Marker adult vs. fetal | 1.987500842 | 1.076238697 | 3.616532831 | 3.470498129 | 2.405945118 | 1.70047288 |
| R88247 | 3 | 2D Marker adult vs. fetal | 9.295559476 | 4.145659051 | 3.807036517 | 3.247695912 | 4.811556959 | 18.08933692 |
| T98812 | 3 | 2D Marker adult vs. fetal | 3.206007578 | 0.45582699 | 7.36008566 | 3.762388177 | 4.629393762 | 2.262352025 |
| AA454858 | 3 | 2D Marker adult vs. fetal | 7.687530164 | 3.130374276 | 3.770230181 | 3.357268094 | 3.90840295 | 11.3903899 |
| N67048 | 2 | 2D Marker adult vs. fetal | 0.182901012 | 0.36769394 | 0.297974786 | 0.30807128 | 0.27564065 | 0.333601513 |
| AA776875 | 2 | 2D Marker adult vs. fetal | 1.088783309 | 0.850650279 | 1.034076531 | 1.167561655 | 0.998364163 | 0.903409454 |
| H51117 | 2 | 2D Marker adult vs. fetal | 0.537932542 | 0.687368102 | 0.574022043 | 0.730299571 | 0.683526033 | 0.743361636 |
| N36174 | 5 | 2D Marker adult vs. fetal | 6.089061165 | 4.481415857 | 5.540191589 | 5.623320599 | 6.588429457 | 4.496005766 |
| AA777187 | 2 | 2D Marker adult vs. fetal | 1.523155704 | 1.077976801 | 2.060524963 | 1.946327352 | 2.299471554 | 1.87592009 |
| R09581 | 8 | 2D Marker adult vs. fetal | 5.890519553 | 3.784033955 | 5.786261707 | 4.733579692 | 4.524489662 | 5.169772778 |
| R16849 | 2 | 2D Marker adult vs. fetal | 1.23979305 | 1.31739538 | 1.989966506 | 0.663751034 | 1.594500153 | 1.415959831 |
| AA884167 | 2 | 2D Marker adult vs. fetal | 1.217420137 | 0.76877689 | 0.848143007 | 1.070264972 | 1.102669725 | 1.097445419 |
| AA136983 | 2 | 2D Marker adult vs. fetal | 1.988298813 | 2.638681789 | 2.540675832 | 2.650969074 | 2.517626988 | 1.836297875 |
| AA488622 | 2 | 2D Marker adult vs. fetal | 1.834368714 | 2.664699416 | 2.916200392 | 2.716178444 | 2.280636291 | 1.670136989 |
| AA699427 | 2 | 2D Marker adult vs. fetal | 1.74393914 | 2.642651634 | 2.705734803 | 2.790851259 | 2.361629453 | 1.978094745 |
| AA490459 | 3 | 2D Marker adult vs. fetal | 6.36833046 | 5.671979456 | 3.680374244 | 4.936768605 | 4.884731709 | 9.255585154 |
| AA626787 | 2 | 2D Marker adult vs. fetal | 1.688259404 | 2.164212705 | 2.115946285 | 1.795197631 | 2.352850319 | 2.121107168 |
| N62179 | 15 | 2D Marker adult vs. fetal | 35.50826346 | 19.11160591 | 33.5269723 | 27.57938931 | 35.32468308 | 29.21398971 |
| N27190 | 16 | 2D Marker adult vs. fetal | 9.750993225 | 7.530891576 | 12.75918957 | 11.05916225 | 12.03535219 | 8.653835849 |
| AA441895 | 16 | 2D Marker adult vs. fetal | 7.177282564 | 5.235719613 | 7.385439482 | 7.014086953 | 8.126515643 | 6.246092446 |
| AA463924 | 16 | 2D Marker adult vs. fetal | 10.41970608 | 7.964616858 | 12.55072628 | 11.42665371 | 12.31290239 | 9.520621452 |
| N78843 | 16 | 2D Marker adult vs. fetal | 7.66464154 | 5.43569693 | 7.61347718 | 7.608367958 | 8.883600715 | 5.827918931 |
| AA629719 | 16 | 2D Marker adult vs. fetal | 14.07113881 | 8.07158282 | 15.07029688 | 13.18842124 | 13.89479922 | 12.78716552 |
| AA404755 | 16 | 2D Marker adult vs. fetal | 10.9079267 | 19.01587747 | 12.27393806 | 10.03671065 | 11.20438161 | 12.29601389 |
| AA459351 | 7 | 2D Marker adult vs. fetal | 3.30738816 | 5.313693876 | 7.122668851 | 3.529883953 | 3.671031365 | 4.776266565 |
| AA488346 | 16 | 2D Marker adult vs. fetal | 9.488691926 | 7.542950447 | 11.21693205 | 9.805337766 | 10.46454533 | 9.180810589 |
| AA427899 | 17 | 2D Marker adult vs. fetal | 13.3365169 | 9.041851128 | 16.97946166 | 12.44206045 | 15.16820039 | 11.53880939 |
| AA453813 | 15 | 2D Marker adult vs. fetal | 23.53014662 | 18.55051373 | 26.13232552 | 24.07754444 | 27.05702114 | 19.41686645 |
| AA397824 | 15 | 2D Marker adult vs. fetal | 28.08351962 | 20.62648968 | 27.02787266 | 26.29257303 | 30.85067131 | 19.80045327 |
| AA633901 | 15 | 2D Marker adult vs. fetal | 34.77984918 | 23.78442887 | 36.12250762 | 35.61488434 | 41.66703684 | 30.60345109 |
| AA181334 | 15 | 2D Marker adult vs. fetal | 27.28075277 | 18.98160489 | 31.18437988 | 27.23129085 | 29.89868199 | 24.05531978 |
| AA292410 | 15 | 2D Marker adult vs. fetal | 31.30959843 | 29.13821092 | 33.60796008 | 35.98378599 | 38.0810897 | 27.93817331 |
| AA253434 | 15 | 2D Marker adult vs. fetal | 27.28836746 | 24.48751872 | 26.021072 | 29.51377393 | 31.81897761 | 24.38068282 |
| AA455058 | 16 | 2D Marker adult vs. fetal | 12.54762444 | 10.90299499 | 12.04192978 | 13.96133558 | 14.93518352 | 10.87358414 |
| R55188 | 21 | 2D Marker adult vs. fetal | 31.12874965 | 12.81904882 | 26.29604395 | 18.72935308 | 27.16189634 | 19.64403686 |
| AA465723 | 0 | 2D Marker adult vs. fetal | 0.626099905 | 0.519829853 | 0.965056345 | 0.801834491 | 0.723063592 | 0.645073541 |
| N49856 | 23 | 2D Marker adult vs. fetal | 58.25496123 | 43.81206927 | 36.30704958 | 58.88107815 | 63.73545411 | 51.53609212 |
| AA455272 | 23 | 2D Marker adult vs. fetal | 78.7226545 | 57.95745288 | 36.30704958 | 70.83386123 | 84.60136833 | 54.29877782 |
| AA459292 | 23 | 2D Marker adult vs. fetal | 84.98013907 | 54.55237528 | 38.30704958 | 78.81031291 | 85.94545998 | 54.78113735 |
| AA878561 | 23 | 2D Marker adult vs. fetal | 91.89023489 | 70.24733836 | 36.30704958 | 85.95771786 | 103.4556246 | 54.83812392 |
| AA772068 | 23 | 2D Marker adult vs. fetal | 100.7022767 | 60.91386332 | 36.30704958 | 95.8034517 | 85.63154687 | 54.85301063 |
| N78821 | 17 | 2D Marker adult vs. fetal | 18.18260113 | 8.900969331 | 17.95968118 | 14.88223883 | 16.81550288 | 14.041252 |
| AA291490 | 21 | 2D Marker adult vs. fetal | 16.15200019 | 12.2589771 | 19.64085664 | 16.98965403 | 18.88134711 | 13.99432924 |
| N46828 | 17 | 2D Marker adult vs. fetal | 16.54096886 | 9.830784212 | 17.53351276 | 13.86763897 | 16.838674726 | 15.02548971 |
| AA150487 | 21 | 2D Marker adult vs. fetal | 15.19512412 | 13.80042191 | 17.95609277 | 14.5094675 | 17.55056275 | 15.10253571 |
| AA282537 | 21 | 2D Marker adult vs. fetal | 17.24244099 | 13.64870386 | 21.82923027 | 18.53902602 | 18.34433042 | 17.95537212 |
| AA707922 | 21 | 2D Marker adult vs. fetal | 29.52423481 | 19.17248102 | 30.42814242 | 29.09439668 | 27.854176 | 25.96068749 |
| AA443638 | 20 | 2D Marker adult vs. fetal | 15.1000594 | 15.15348782 | 17.88558853 | 15.7697112 | 18.94151907 | 14.61633105 |
| W73892 | 19 | 2D Marker adult vs. fetal | 53.62002013 | 39.02380655 | 36.30704958 | 56.67674826 | 53.82921611 | 46.25990911 |
| N70734 | 19 | 2D Marker adult vs. fetal | 50.1395528 | 37.01986341 | 36.30704958 | 48.48335304 | 47.57551429 | 45.25023946 |
| H57136 | 13 | 2D Marker adult vs. fetal | 10.27199646 | 9.124487788 | 12.5238108 | 9.80585148 | 11.12532179 | 10.90999216 |
| AA700414 | 29 | 2D Marker adult vs. fetal | 72.81271834 | 37.61546462 | 36.30704958 | 68.67343991 | 62.25292659 | 54.4411949 |
| W65461 | 7 | 2D Marker adult vs. fetal | 5.476839239 | 5.067442981 | 5.426887725 | 6.079126118 | 6.4014634348 | 5.626362584 |
| AAA36584 | 15 | 2D Marker adult vs. fetal | 23.02941313 | 15.24754038 | 25.73740515 | 22.61556954 | 22.7981159 | 19.91483801 |
| AA029042 | 9 | 2D Marker adult vs. fetal | 8.022234644 | 7.311596005 | 8.895662277 | 9.479217739 | 9.605485934 | 7.544837461 |
| AA427725 | 9 | 2D Marker adult vs. fetal | 12.69294864 | 7.655536142 | 13.67326812 | 4.261155334 | 12.4290971 | 12.81248352 |
| N51280 | 9 | 2D Marker adult vs. fetal | 6.632333815 | 5.874595041 | 7.385166272 | 7.520608106 | 7.785748506 | 5.963530521 |
| AA281347 | 8 | 2D Marker adult vs. fetal | 5.089780613 | 4.640487592 | 4.9415594344 | 5.45251025 | 6.288204512 | 4.490066004 |
| AA402960 | 8 | 2D Marker adult vs. fetal | 4.965150621 | 4.50182924 | 6.596223821 | 2.242032928 | 4.880356598 | 4.839655851 |
| N98485 | 9 | 2D Marker adult vs. fetal | 7.125405927 | 6.544129951 | 7.7721864 | 7.556753899 | 8.479066808 | 6.411666818 |
| AA490209 | 7 | 2D Marker adult vs. fetal | 5.803194729 | 5.024903 | 4.546705412 | 6.046321655 | 6.500609214 | 4.920009122 |
| W61361 | 9 | 2D Marker adult vs. fetal | 7.384750912 | 7.335298925 | 11.10935518 | 8.8100027 | 9.421761776 | 6.881790584 |
| N51018 | 7 | 2D Marker adult vs. fetal | 5.488100802 | 38.59262312 | 10.6998308 | 6.032584586 | 5.216849242 | 17.32422488 |
| AA455281 | 7 | 2D Marker adult vs. fetal | 6.998082782 | 9.317433876 | 8.495460011 | 7.352959068 | 7.938571107 | 8.110370507 |
| W69471 | 7 | 2D Marker adult vs. fetal | 7.215022797 | 8.148565459 | 8.197656911 | 5.32000885 | 4.956859017 | 13.55485747 |
| AA486321 | 7 | 2D Marker adult vs. fetal | 6.616843406 | 3.688421667 | 10.1148224 | 5.538922361 | 5.404776773 | 6.062776617 |
| AA458982 | 7 | 2D Marker adult vs. fetal | 6.961286958 | 6.280975266 | 6.441469774 | 7.072922475 | 7.646598009 | 6.402438243 |
| AA442095 | 7 | 2D Marker adult vs. fetal | 10.53368867 | 6.969251828 | 9.035104314 | 8.530732052 | 8.715924875 | 7.353607298 |
| N99003 | 7 | 2D Marker adult vs. fetal | 7.777346527 | 5.123406597 | 7.271360835 | 7.694076299 | 8.015056524 | 6.947984869 |
| AA609284 | 7 | 2D Marker adult vs. fetal | 5.203527631 | 6.725228727 | 5.962252816 | 5.392872692 | 5.486984016 | 6.647332466 |
| AA195036 | 12 | 2D Marker adult vs. fetal | 4.739031812 | 5.630223489 | 6.168419327 | 3.952137125 | 6.299183956 | 4.511434885 |
| AA478268 | 5 | 2D Marker adult vs. fetal | 4.587623085 | 4.319668521 | 6.07446736 | 5.321094819 | 5.705783899 | 4.273131909 |
| AA608583 | 13 | 2D Marker adult vs. fetal | 8.710213419 | 6.633631914 | 10.89267006 | 9.897803204 | 10.76405611 | 7.594364628 |
| AA480435 | 13 | 2D Marker adult vs. fetal | 10.66603107 | 6.878393398 | 11.70702321 | 9.862860466 | 10.62920807 | 10.9035389 |
| AA505046 | 13 | 2D Marker adult vs. fetal | 6.175908969 | 7.774740452 | 10.84632581 | 7.657584613 | 7.632328468 | 5.432221512 |
| AA487893 | 13 | 2D Marker adult vs. fetal | 6.28913391 | 7.086629819 | 10.38317033 | 8.186637919 | 8.677781639 | 6.646527414 |
| AA292226 | 12 | 2D Marker adult vs. fetal | 6.978311765 | 9.498379167 | 9.999757753 | 8.245173408 | 8.45533271 | 7.944255354 |
| H87106 | 9 | 2D Marker adult vs. fetal | 8.511773166 | 8.306766068 | 8.639728411 | 8.688270747 | 9.817268292 | 7.409517924 |
| W96450 | 12 | 2D Marker adult vs. fetal | 6.333304604 | 6.11454874 | 10.93854816 | 8.295025536 | 8.501848685 | 6.781838425 |
| N33331 | 13 | 2D Marker adult vs. fetal | 12.01197758 | 9.753283045 | 14.0583162 | 10.92456794 | 13.45540044 | 19.89513482 |
| AA405800 | 12 | 2D Marker adult vs. fetal | 6.121396479 | 5.223320588 | 7.721500548 | 7.718106658 | 6.462547598 | 5.753462094 |
| T51539 | 12 | 2D Marker adult vs. fetal | 6.489900146 | 6.570558251 | 9.469029997 | 8.820264288 | 9.194419114 | 5.883142749 |
| N59764 | 12 | 2D Marker adult vs. fetal | 7.600540659 | 4.756724822 | 8.39191538 | 7.279906425 | 8.135204655 | 6.251234677 |
| AA521346 | 11 | 2D Marker adult vs. fetal | 18.0187463 | 11.98813991 | 20.04539147 | 17.66437285 | 19.72793582 | 15.44668566 |
| AA428551 | 11 | 2D Marker adult vs. fetal | 11.81379838 | 9.132344514 | 13.57703545 | 12.536771 | 14.26115078 | 11.3197892 |
| AA489383 | 11 | 2D Marker adult vs. fetal | 10.07196453 | 6.40710185 | 10.63654538 | 9.115267349 | 9.345806882 | 8.56845107 |
| AA490172 | 11 | 2D Marker adult vs. fetal | 8.374766147 | 1.415297485 | 17.38713256 | 12.09049918 | 8.290346893 | 6.184780432 |
| AA504477 | 12 | 2D Marker adult vs. fetal | 5.417891216 | 3.85972235 | 6.090353431 | 5.209247321 | 5.829042051 | 4.969815506 |

| Accesion | H1 V2 t0 | H1 V2 t2d | H1 V2 t4d | H2 P1 3D | H2 P1 2D | H3 F P4 t0d | H3 F P4 t2d | H3 F P4 t7d |
|---|---|---|---|---|---|---|---|---|
| AA283693 | 3.60E−02 | 7.00E−02 | 0.322383183 | 0.447391374 | 0.257739798 | 0.143513725 | 0.213829818 | 5.224212686 |
| AA845156 | 1.28342518 | 1.863140939 | 1.384248489 | 1.041642418 | 1.859400083 | 1.332668422 | 1.018759388 | 5.129921131 |
| R52548 | 24.22599845 | 23.2864338 | 18.34158303 | 25.57074708 | 22.01795031 | 27.50011776 | 26.82422658 | 0.455256887 |
| T67128 | 0.104460037 | 3.43E−02 | 1.158151225 | 0.122945522 | 0.194757907 | 0.263409159 | 0.266372201 | 15.92056233 |
| AA845015 | 5.971265034 | 6.563146942 | 3.845471328 | 4.218022136 | 6.266907187 | 4.459497525 | 3.113814791 | 23.77610407 |
| AA937895 | 0.452860231 | 0.364695951 | 1.498136505 | 1.34894881 | 1.524476699 | 1.004544129 | 0.332861119 | 23.03713043 |
| AA844998 | 11.26686885 | 9.619376872 | 4.163473688 | 8.673344954 | 10.804701 | 11.6741356 | 8.48774821 | 0.687222458 |
| AA844518 | 48.83476799 | 54.52879325 | 40.56584843 | 54.89200718 | 49.39056409 | 42.93573187 | 47.44855774 | 6.960856259 |
| AA894557 | 49.93664852 | 44.42230199 | 49.4951755 | 65.92881949 | 60.33602671 | 39.11417155 | 46.47247102 | 1.451948868 |
| AA872001 | 54.20005466 | 64.75076884 | 65.42329615 | 62.45668946 | 67.24659793 | 53.73423923 | 63.31563058 | 1.313345062 |
| H09590 | 0.653737608 | 0.390597399 | 1.521400852 | 0.559410558 | 0.287251416 | 0.132045049 | 5.76E−02 | 9.888630279 |
| AA888278 | 4.209025695 | 6.432026011 | 1.564621676 | 2.03670347 | 3.658833325 | 5.897415558 | 5.585056007 | 4.869119818 |
| AA490855 | 7.139496238 | 7.480509788 | 2.402406359 | 2.11287386 | 3.291155604 | 4.163992221 | 4.626992228 | 4.040992653 |
| H05820 | 0.250153138 | 0.2218924 | 0.884601448 | 0.443767203 | 0.853117504 | 0.577197042 | 0.227069034 | 8.569006814 |
| N57766 | 0.161049809 | 1.029762957 | 0.860155011 | 1.160409378 | 0.696379763 | 1.945055757 | 1.505490705 | 18.21699443 |
| AA873885 | 0.576137629 | 0.747123686 | 1.364947624 | 0.484042151 | 0.351548888 | 2.300286033 | 1.86211811 | 31.75585388 |
| AA878880 | 0.214554665 | 0.751859199 | 1.555155015 | 0.580037834 | 0.413262131 | 0.487069801 | 0.493477276 | 17.65353573 |
| R54818 | 0.646985459 | 0.409947934 | 0.199952191 | 0.325728665 | 0.57824012 | 0.478785614 | 0.148018732 | 5.693624857 |
| AA458830 | 10.09192142 | 11.58519602 | 4.33841323 | 6.048695063 | 5.824480196 | 13.79679438 | 10.66742276 | 10.18586889 |
| W37884 | 2.917746898 | 4.089689479 | 1.262910319 | 1.511275797 | 1.980865808 | 5.197874299 | 6.453757844 | 5.811360752 |
| N63192 | 3.165162737 | 4.106218227 | 1.558423149 | 1.5903832 | 2.110133253 | 5.489587577 | 6.303860434 | 5.163746261 |
| R55789 | 5.839714648 | 7.877144785 | 2.228165523 | 3.701220707 | 3.164509514 | 3.916307321 | 3.178401276 | 3.905915697 |
| R56871 | 8.541201281 | 9.565561925 | 5.095438247 | 5.327868943 | 7.13865299 | 8.798222752 | 9.999533942 | 1.720941975 |
| AA448659 | 6.40985601 | 8.062945347 | 2.837008591 | 2.740282482 | 3.524896196 | 8.26644815 | 9.131700385 | 6.724601997 |
| AA235388 | 5.495213871 | 7.128779744 | 1.810297945 | 3.365604514 | 4.797167927 | 5.188848193 | 3.719920825 | 3.55603538 |
| W37769 | 7.944164954 | 9.806778833 | 2.976028459 | 4.717147657 | 6.792587093 | 10.44168598 | 10.83208914 | 8.961263558 |
| AA421701 | 8.998110252 | 11.20923244 | 3.344552795 | 4.634853853 | 5.630305124 | 9.009982779 | 7.214242967 | 8.676253864 |
| NB1029 | 12.53804212 | 11.46996906 | 3.601513562 | 9.079241362 | 7.051304119 | 8.667406893 | 7.19394857 | 6.694763747 |
| AA644128 | 11.11148757 | 10.68939675 | 3.391349693 | 5.57092524 | 5.21033759 | 8.326957952 | 9.801034583 | 8.395745244 |
| N26536 | 12.17742976 | 6.963934008 | 2.083996724 | 2.539664871 | 3.338342813 | 6.091759705 | 5.481627936 | 4.663694738 |
| AA890663 | 13.06099681 | 8.389833107 | 0.735677843 | 3.645361823 | 5.830896283 | 10.14275452 | 7.73405205 | 8.622524146 |
| AA405987 | 10.80261435 | 12.021725 | 2.747874952 | 3.870971862 | 6.423478204 | 9.717748938 | 10.38278845 | 8.452850767 |
| AA888182 | 1.015717155 | 1.005037796 | 10.31208553 | 1.080891115 | 0.620362547 | 0.742540888 | 0.217171056 | 0.963761651 |
| H09730 | 11.63478455 | 9.263731897 | 2.426058114 | 3.698744788 | 6.251078878 | 11.24128172 | 10.27180594 | 8.218265778 |
| AA285155 | 12.85800077 | 9.831563278 | 3.688749895 | 5.365619531 | 5.184975167 | 7.402395869 | 7.379800537 | 6.209664883 |
| AA873351 | 0.470607591 | 0.631814307 | 7.979382806 | 0.79673277 | 0.500876851 | 0.326537695 | 0.408343693 | 0.899376594 |
| H12320 | 0.282438829 | 1.169093316 | 7.275998896 | 1.197723988 | 0.103345305 | 0.205188914 | 0.219791049 | 0.757004021 |
| AA856558 | 0.397739468 | 0.400975764 | 8.510141689 | 1.29264139 | 0.857904175 | 0.471466936 | 0.271477848 | 0.790926029 |
| R43581 | 0.381477112 | 0.761735816 | 6.064087362 | 1.931094763 | 0.818248288 | 0.397440561 | 0.456001183 | 0.419248288 |
| AA633768 | 0.760761379 | 0.497766033 | 5.606806863 | 0.633870185 | 0.393352577 | 0.351794529 | 0.601192682 | 1.14487198 |
| AA496880 | 0.493894518 | 0.572564726 | 6.056796145 | 0.653312475 | 0.514470842 | 0.502539979 | 0.501308175 | 1.212402498 |
| AA625632 | 0.842550697 | 0.850281232 | 10.68487135 | 1.661720166 | 0.695569959 | 0.498447008 | 0.274386994 | 1.69499 |
| R40850 | 0.394670064 | 0.363742341 | 4.967702878 | 1.362523305 | 0.380406218 | 0.141276114 | 0.169003657 | 0.619590829 |
| AA486072 | 1.063850678 | 3.668838298 | 12.43527006 | 3.538640747 | 0.712197565 | 1.714315274 | 1.299858757 | 2.072066795 |
| N80129 | 0.10312387 | 0.42006458 | 10.27394074 | 0.364964803 | 0.173871475 | 3.53E−02 | 9.69E−02 | 1.445787697 |
| T67270 | 0.706906482 | 1.1288523 | 9.031533244 | 1.747487433 | 1.676363321 | 0.971529338 | 1.02792629 | 1.159935914 |
| AA776304 | 1.05308668 | 1.608943664 | 7.115065643 | 1.060601139 | 1.428674622 | 1.215371395 | 1.026193827 | 2.050698416 |
| AA64743 | 0.720584255 | 1.207641114 | 6.709648148 | 1.123344154 | 0.916034946 | 0.548390406 | 0.6724641 | 1.948529432 |
| AA663983 | 2.040575612 | 0.913188385 | 5.16313531 | 1.007867654 | 1.323649464 | 2.135989003 | 8.32E−02 | 3.811870062 |
| AA634008 | 1.359339921 | 1.036497507 | 13.34177496 | 1.724576212 | 0.853238694 | 1.024786523 | 1.068524094 | 1.956193928 |
| AA683050 | 1.013489887 | 1.038528355 | 15.81952721 | 1.58843476 | 0.496740614 | 0.558453728 | 0.815020862 | 1.866191062 |
| AA775874 | 0.246852476 | 0.424685862 | 5.143824512 | 0.974500161 | 0.529437509 | 0.290250898 | 0.38554521 | 1.21495112 |
| AA029934 | 10.39261667 | 6.173829216 | 3.256015168 | 2.937213126 | 3.76308931 | 6.716135076 | 4.994432581 | 4.353783816 |
| AA872397 | 46.00745138 | 39.17088563 | 36.04470776 | 60.24320179 | 47.48372041 | 40.84667624 | 48.51971658 | 25.61671577 |
| AA428195 | 5.50376757 | 5.85445104 | 2.298660486 | 2.154530299 | 3.416182085 | 4.515305124 | 4.564251077 | 3.09255509 |
| AA478724 | 2.441015531 | 4.011719078 | 1.703691212 | 1.339633614 | 2.575865932 | 1.573760187 | 1.247433101 | 2.275822402 |
| T40541 | 0.275153908 | 0.545092967 | 3.902618974 | 0.963575617 | 9.51E−02 | 8.50E−02 | 3.92E−02 | 1.06319634 |
| N33214 | 0.13076449 | 0.257442727 | 1.248745094 | 1.214456176 | 0.147732522 | 0.479395409 | 5.23E−02 | 1.778833698 |
| W69399 | 0.345223646 | 0.364082141 | 1.11200994 | 0.651631382 | 0.156264888 | 4.35E−02 | 5.26E−02 | 1.090858575 |
| H85454 | 0.177423187 | 0.164239157 | 0.25952362 | 0.278813589 | 0.207718277 | 0.192428939 | 0.293284362 | 0.980664216 |
| T71284 | 3.25E−02 | 4.43E−02 | 4.34E−03 | 0.114965169 | 0.174872171 | 0.141660059 | 0.315197363 | 0.637643567 |
| N95418 | 7.17E−02 | 4.56E−02 | 0.17695605 | 0.230927882 | 2.96E−02 | 3.19E−02 | 1.92E−02 | 0.409050075 |
| AA430675 | 29.3586379 | 28.49610318 | 30.86231132 | 32.28482203 | 44.49536923 | 33.27001036 | 32.81546787 | 20.72524354 |
| AA682851 | 46.90243645 | 42.6704861 | 49.46272263 | 54.93185512 | 60.11034283 | 37.00532862 | 37.62408166 | 23.34221611 |
| AA427433 | 4.590770914 | 6.491096293 | 2.119161449 | 2.512849593 | 3.622778825 | 5.405043355 | 5.599238453 | 5.427644127 |
| AA100296 | 39.44423876 | 56.23942898 | 41.49374024 | 50.80500421 | 43.55754452 | 57.54473062 | 53.89058071 | 27.20955224 |
| AA070997 | 44.50541019 | 42.47813824 | 34.76574485 | 44.39487164 | 50.26187697 | 33.36357052 | 29.205598 | 27.82261502 |
| R27585 | 35.33261308 | 33.39411146 | 29.7220802 | 41.54702124 | 41.6509187 | 34.23348926 | 26.42844313 | 25.01170127 |
| N71628 | 29.62587768 | 25.73280775 | 27.314568 | 42.44280692 | 32.44686223 | 28.50781759 | 31.40872062 | 17.11695396 |
| AA484566 | 36.70082765 | 34.8718366 | | 38.3684724 | 32.25023119 | 37.7985424 | 98.44938631 | 22.42777135 |
| AA043228 | 35.07501988 | 35.26683595 | 39.99311042 | 38.42578454 | 28.64861089 | 33.09819082 | 42.01213318 | 20.20000016 |
| AA478273 | 24.39470164 | 24.77474479 | 23.18365645 | 21.88455412 | 23.61056895 | 34.11709903 | 37.1720164 | 22.29749728 |
| H05618 | 0.287634172 | 0.126355776 | 0.103328665 | 0.53496355 | 0.113769783 | 0.103142991 | 0.103572238 | 0.411960124 |
| AA405562 | 13.78665714 | 15.04195781 | 4.06566807 | 10.07318456 | 10.12150087 | 9.869201884 | 10.11315283 | 9.063379829 |
| AA147043 | 10.06577793 | 10.38848924 | 8.480460276 | 9.593900583 | 10.3223246 | 9.358593384 | 9.251590954 | 7.969342929 |
| AA035384 | 12.04070004 | 12.92252006 | 15.89036927 | 11.03937435 | 13.98924616 | 10.56026075 | 7.850369977 | 8.464122084 |
| R60150 | 9.633059103 | 11.34413022 | 3.55905762 | 5.14548369 | 8.554543073 | 8.219606806 | 10.11952936 | 7.877309364 |
| N64051 | 20.20297 | 17.93454359 | 6.059875542 | 10.65425592 | 11.69250488 | 14.02690246 | 13.40703529 | 11.39979162 |
| AA405748 | 11.19414229 | 11.22331463 | 8.725898139 | 10.22004433 | 9.939101581 | 9.624639345 | 6.736761694 | 7.207953341 |
| AA461110 | 11.42516439 | 11.54464774 | 11.22768955 | 11.5865327 | 9.335135467 | 1.599312321 | 1.604370138 | 0.742502214 |
| AA845167 | 15.24112672 | 15.33748563 | 10.34294903 | 10.49210118 | 9.895195623 | 11.54052428 | 13.14844812 | 9.845307435 |
| AA443118 | 15.71033269 | 12.21523831 | 7.066371438 | 9.278769927 | 12.62454607 | 12.73679044 | 11.89501447 | 11.26385022 |
| N92319 | 0.68013854 | 0.462694318 | 0.441158367 | 0.601101086 | 0.511427071 | 0.795108656 | 0.113909679 | 1.02641734 |
| AA187148 | 11.23551547 | 10.38626391 | 7.898742343 | 8.98515106 | 8.886781354 | 9.776711665 | 9.067245371 | 5.447187287 |
| AA253413 | 5.375893098 | 6.14287559 | 2.014829497 | 2.303548324 | 3.272998057 | 3.697482133 | 3.356931434 | 3.421688731 |
| AA045701 | 16.07206256 | 16.18438115 | 15.35025693 | 11.75303107 | 17.53371006 | 21.28041445 | 23.57251468 | 18.29553133 |
| AA164562 | 13.50161337 | 11.85115038 | 11.90157085 | 12.70121113 | 13.97404342 | 19.51320109 | 20.14729058 | 12.69772915 |
| AA486357 | 19.81244564 | 19.08483294 | 9.978745784 | 13.47424147 | 16.26643795 | 17.39386742 | 17.49834817 | 12.94888047 |
| AA180742 | 21.52699749 | 16.35007638 | 6.426135031 | 15.13185304 | 13.19450881 | 15.59400571 | 17.9357522 | 13.28038761 |
| AA454743 | 14.91925254 | 13.9599813 | 8.65144627 | 12.90834328 | 11.96847998 | 14.68622205 | 15.68194498 | 11.59505554 |
| AA437226 | 14.85387781 | 17.10133551 | 12.22946701 | 13.14384264 | 15.14400311 | 17.52701687 | 17.25793589 | 10.50190917 |
| AA458849 | 2.017166995 | 2.34556391 | 1.907000939 | 2.12162749 | 2.328337653 | 2.492978456 | 2.054641085 | 2.159670081 |
| AA604891 | 6.821939878 | 6.718604337 | 7.304547885 | 2.689809619 | 4.718893627 | 4.373418086 | 5.049991825 | 7.871176277 |
| AA609655 | 33.01277519 | 29.42862393 | 30.32802604 | 37.60777502 | 42.07314192 | 25.19427243 | 25.92346393 | 17.03793176 |
| AA599158 | 7.858583214 | 7.185543001 | 6.300783712 | 9.094322938 | 6.597531463 | 10.27562393 | 10.16458119 | 6.424661999 |
| AA052932 | 0.253881413 | 0.171034437 | 0.788537559 | 1.088792526 | 0.303760633 | 0.25364904 | 0.134116019 | 0.728786605 |
| AA769328 | 0.343955354 | 0.254618771 | 0.596171232 | 0.419246635 | 0.43081793 | 0.595702083 | 0.354185573 | 1.725014554 |
| AA129537 | 8.960638915 | 7.77671267 | 8.311570259 | 11.4292329 | 11.96830695 | 14.65748429 | 13.25871143 | 8.624849205 |
| AA486209 | 0.130869696 | 4.19E−02 | 0.222978133 | 0.376314559 | 0.117276599 | 0.113920001 | 8.25E−02 | 0.77617085 |
| H39016 | 0.14027571 | 0.094249721 | 0.182618167 | 0.2492443 | 0.135370399 | 0.271580421 | 0.149412484 | 1.0709987 |
| AA464217 | 0.226422488 | 7.45E−02 | 1.63231874 | 0.357023921 | 0.235657222 | 0.260977316 | 5.92E−02 | 1.998287174 |
| T95053 | 0.506118797 | 0.705352124 | 2.701833003 | 0.643138309 | 0.324882792 | 0.304836811 | 0.263668115 | 1.188075083 |
| AA454640 | 0.220375821 | 0.171878877 | 0.622604377 | 0.509056108 | 7.62E−02 | 3.06E−02 | 4.12E−02 | 0.615361756 |
| AA448400 | 0.43389112 | 0.405349064 | 0.482918962 | 2.543019921 | 5.42E−02 | 7.67E−02 | 5.68E−02 | 0.744503106 |
| H13691 | 38.06839644 | 45.61749341 | 23.18204722 | 19.59908539 | 32.30677452 | 35.30376824 | 30.58992766 | 0.559437392 |
| AA132086 | 0.368363187 | 0.158208065 | 0.357001431 | 0.998742637 | 0.108277685 | 0.210123178 | 5.56E−02 | 1.097353089 |
| AA488073 | 0.223040973 | 0.324590132 | 1.411227979 | 0.464995516 | 1.253748511 | 1.167155752 | 0.478968407 | 1.88302538 |
| N40945 | 0.262201912 | 0.237321497 | 0.257785251 | 0.258650997 | 0.597559834 | 0.252450904 | 0.128562307 | 1.019389108 |
| R55705 | 0.137145705 | 9.39E−02 | 0.38327564 | 0.365029393 | 8.79E−02 | 2.14E−02 | 1.16E−02 | 0.572340722 |
| H50114 | 0.149230356 | 0.114329742 | 0.542033448 | 0.613679831 | 5.55E−02 | 1.78E−02 | 1.81E−02 | 0.601295897 |
| AA452841 | 0.241515566 | 0.336987054 | 0.216412064 | 0.47285925 | 0.332567526 | 0.409686162 | 0.241219124 | 1.10120583 |
| W73790 | 0.234487599 | 0.215971925 | 0.741717434 | 0.468556893 | 0.327293291 | 8.49E−02 | 0.104656358 | 0.735173064 |
| N30302 | 26.09274291 | 22.41827113 | 23.15567368 | 29.55256655 | 21.88167764 | 23.38219958 | 20.81894087 | 13.76515858 |
| AA291556 | 0.384633298 | 0.205779366 | 0.659149864 | 0.446933867 | 0.146800827 | 0.145065316 | 0.356438595 | 1.601652315 |
| AA598510 | 3.61E−02 | 2.40E−02 | 0.108970915 | 0.102809842 | 7.85E−02 | 5.60E−02 | 0.037519507 | 0.436123593 |
| AA453787 | 0.512016161 | 0.518525655 | 0.584688061 | 0.68944295 | 0.284454929 | 6.55E−02 | 5.59E−02 | 0.634550941 |
| H05655 | 0.163032728 | 7.17E−02 | 0.27016244 | 0.166433782 | 10.00E−02 | 3.94E−02 | 9.58E−02 | 0.498009645 |
| AA419177 | 36.78866172 | 36.69047817 | 43.9286988 | 43.54178839 | 51.77202168 | 45.8338345 | 51.06623927 | 33.61266646 |
| AA458807 | 33.10376447 | 30.88801173 | 26.67225549 | 34.5554514 | 37.93370032 | 30.31855172 | 39.9630798 | 23.50761944 |
| AA293218 | 26.4028529 | 35.80834176 | 26.15447974 | 33.64819217 | 31.7917979 | 29.84313354 | 26.41862616 | 17.3599263 |
| W44860 | 25.75715266 | 24.52077453 | 4.553792207 | 26.21041688 | 26.01117112 | 28.71589625 | 29.44026693 | 18.26263026 |
| AA629862 | 34.76420835 | 33.86130994 | 30.29052693 | 26.75522669 | 33.11708939 | 36.29104725 | 43.93948571 | 27.19603411 |
| AA447674 | 0.180546405 | 0.126712928 | 0.229464734 | 0.259752602 | 0.109955437 | 8.90E−02 | 6.90E−02 | 0.339734269 |
| T52484 | 0.281812925 | 9.64E−02 | 0.826718721 | 0.565508854 | 0.485893021 | 0.432859528 | 0.061805942 | 0.992986281 |
| AA496810 | 9.34E−02 | 7.12E−02 | 6.46E−04 | 0.295018299 | 8.08E−02 | 2.97E−02 | 2.19E−02 | 0.446821097 |
| AA486233 | 0.340410827 | 0.117869448 | 1.53377804 | 0.356012024 | 0.538022414 | 0.203021316 | 0.156983718 | 1.084397595 |
| AA079775 | 0.439060283 | 0.619002395 | 0.9808177 | 1.409206936 | 0.296042275 | 0.11668949 | 0.105478767 | 0.789070453 |
| W73889 | 0.126123783 | 0.64173358 | 2.015296373 | 0.918137122 | 2.418583185 | 8.49E−02 | 0.815254724 | 2.695275623 |
| R50337 | 2.97E−02 | 2.35E−02 | 0.295641965 | 0.133290557 | 2.04E−02 | −3.11E−05 | 1.07E−02 | 0.398363748 |
| R55046 | 3.121779976 | 3.583304812 | 1.420346967 | 1.371280523 | 2.205699254 | 3.267553012 | 3.02014176 | 3.938693805 |
| R46821 | 2.400280379 | 2.776566441 | 1.281366833 | 1.119135092 | 2.939835087 | 4.354026018 | 3.384328112 | 4.379616557 |
| R87763 | 0.132976244 | 0.106549764 | 0.245268993 | 1.030976231 | 8.11E−02 | 0.130698981 | 5.81E−02 | 1.214742481 |
| H89583 | 0.18904047 | 0.101052954 | 0.236746017 | 0.351564341 | 9.66E−02 | 0.145392235 | 3.87E−02 | 0.781599712 |
| R56040 | 2.785909659 | 2.969666922 | 10.5096934 | 1.626253524 | 1.779842426 | 1.69343824 | 1.550682609 | 3.471096224 |
| AA922705 | 0.161006175 | 0.118245397 | | 0.426100265 | 0.119115471 | 3.86E−02 | 0.02671598 | 1.293134938 |
| AA487571 | 0.135236965 | 0.118211417 | 0.99308562 | 0.328940183 | 8.72E−02 | 2.81E−02 | 3.65E−02 | 0.965119113 |
| AA402440 | 0.406400795 | 0.454162321 | 1.143777395 | 0.734135081 | 0.607411285 | 0.139247414 | 9.06E−02 | 1.2521297 |
| H29521 | 0.292621042 | 0.214822388 | 0.371852071 | 0.98238363 | 0.127904929 | 0.150473865 | 0.25566945 | 0.418395022 |
| AA490911 | 21.06562623 | 18.4520925 | 18.3142956 | 15.8556682 | 20.57635735 | 22.99182529 | 24.71655205 | 12.4435305 |
| AA486082 | 23.15717253 | 22.55169547 | 22.5377778 | 21.98212686 | 26.33492887 | 25.7856523 | 27.61639941 | 17.1571581 |
| AA678065 | 43.1549231 | 49.75180877 | 44.95138637 | 30.65381761 | 40.86775329 | 42.55581559 | 44.82447844 | 19.44908824 |
| R43509 | 27.41149216 | 22.05072953 | 25.03729443 | 27.50714533 | 32.1008748 | 21.09382826 | 22.18972333 | 15.16640501 |
| N57653 | 31.9568203 | 30.32164198 | 22.03248851 | 27.95239629 | 27.75233699 | 28.33450066 | 32.56698428 | 19.23600632 |
| AA670955 | 27.613137 | 30.15379516 | 27.7763484 | 26.77163594 | 19.30516442 | 23.91926132 | 25.65098101 | 15.57144585 |
| R14692 | 0.210407505 | 6.26E−02 | 1.409022733 | 0.795196983 | 0.677281805 | 0.581851815 | 0.140894744 | 17.15858056 |
| AA488979 | 21.80183287 | 22.74108924 | 21.96333121 | 15.76906128 | 18.73408294 | 20.2709434 | 23.79553223 | 13.91426041 |
| AA443630 | 0.100651283 | 9.85E−02 | 4.85E−03 | 0.234647143 | 3.12E−02 | 2.15E−02 | 1.05E−02 | 9.71E−02 |
| AA027840 | 11.00979974 | 13.09339183 | 11.2816884 | 9.089927285 | 14.0930363 | 14.886840831 | 16.74903887 | 9.439049175 |
| AA458830 | 11.37202854 | 11.65474155 | 6.607372436 | 7.389629431 | 10.78058971 | 10.57865908 | 11.80737106 | 9.428876044 |
| AA459015 | 22.13388102 | 21.94307144 | 21.95512611 | 28.03858305 | 24.90291416 | 27.82736689 | 24.89782479 | 18.16821948 |
| AA074446 | 29.21687173 | 22.22581916 | 16.8371288 | 24.23743962 | 26.33743534 | 26.81890595 | 25.69413696 | 18.03496456 |
| AA027042 | 24.22764975 | 23.72047212 | 25.83781841 | 23.88440587 | 24.95924388 | 21.01386997 | 22.28219305 | 15.37688987 |
| AA629923 | 20.68133909 | 17.84071537 | 18.57653108 | 22.21025049 | 25.34003259 | 22.25297762 | 25.08441753 | 12.30816863 |
| AA460830 | 18.35934471 | 16.37670009 | 16.4942895 | 20.66510843 | 21.27569752 | 22.24115944 | 20.1395633 | 14.54358761 |
| AA454218 | 4.705025791 | 5.029967147 | 5.2176009 | 6.528355913 | 6.43699759 | 3.661787064 | 3.143045096 | 3.120347736 |
| AA046523 | 6.972538399 | 8.239721684 | 8.685210367 | 9.087650516 | 10.97162504 | 10.44142681 | 9.712940248 | 6.973860192 |
| R51346 | 9.268892528 | 11.49106845 | 6.177160828 | 7.887574405 | 8.018087028 | 10.44726109 | 9.783512947 | 0.215474051 |
| AA029964 | 7.968348913 | 10.02474618 | 7.998306197 | 7.953245463 | 6.008361088 | 14.22552591 | 13.76159965 | 9.150938402 |
| AA489219 | 3.977503262 | 4.357159927 | 3.843713092 | 6.387649039 | 5.182503022 | 4.084539228 | 4.217440316 | 3.929360037 |
| AA043133 | 7.10915371 | 6.657783457 | 7.232430397 | 9.095892814 | 7.495998702 | 8.296677078 | 7.86458314 | 5.986265513 |
| AA812973 | 31.52455964 | 28.41615616 | 25.69771578 | 32.72534183 | 34.85110642 | 31.04591265 | 32.76410913 | 19.46276288 |
| AA453471 | 9.736208184 | 11.1890541 | 2.985624257 | 6.304114807 | 6.507046353 | 13.54093424 | 11.82320488 | 11.84074045 |
| AA284693 | 22.56815563 | 31.15842013 | 22.14580038 | 30.90223481 | 26.73160123 | 32.27184876 | 29.98195912 | 19.10255765 |
| N90281 | 6.43341783 | 5.959020565 | 4.766657998 | 6.027996022 | 4.863654532 | 5.006421597 | 5.844930445 | 3.835280867 |
| AA629542 | 7.506542166 | 7.498548839 | 5.126718959 | 5.758458157 | 5.628204957 | 10.92011362 | 10.66563175 | 8.830974583 |
| AA679345 | 11.61400851 | 12.1850373 | 0.318260632 | 7.086444187 | 10.6336761 | 12.99915052 | 14.28658079 | 11.11062364 |
| H37774 | 2.83E−02 | 5.37E−02 | 0.455840137 | 0.23068029 | 0.027217407 | 8.89E−03 | 7.83E−03 | 0.143799652 |
| T97181 | 1.876643541 | 0.587861517 | 0.977072756 | 0.374576034 | 1.401186113 | 0.980966491 | 0.307497525 | 0.977649837 |
| AA454879 | 0.128788844 | 5.49E−02 | 0.642103913 | 0.314369938 | 0.171773759 | 6.95E−02 | 6.83E−02 | 0.364117574 |
| AA147640 | 11.10684686 | 11.11067304 | 4.334390147 | 6.745732625 | 7.498713228 | 9.714345375 | 10.05962462 | 9.682475093 |
| AA767429 | 13.9665151 | 17.25513268 | 9.348363447 | 14.61003519 | 13.1757222 | 18.11092619 | 17.6813815 | 12.40577438 |
| AA490991 | 19.30297964 | 18.58197931 | 18.34934113 | 17.62227921 | 19.95904418 | 22.8372929 | 20.13821173 | 14.51945376 |
| AA422058 | 31.53896803 | 26.94893157 | 19.67019378 | 29.90401606 | 24.82963178 | 26.86419627 | 30.12270888 | 20.6779599 |
| N66208 | 15.78244935 | 17.89542247 | 17.81439029 | 14.63969059 | 15.54037985 | 19.14257856 | 19.46042164 | 12.13187951 |
| AA630776 | 16.29858697 | 16.75035086 | 16.12331467 | 15.92787633 | 17.52684563 | 20.53155822 | 17.53930107 | 12.98947212 |
| AA827287 | 39.31448991 | 30.4334384 | 11.88952147 | 17.38709762 | 17.86731786 | 24.85586178 | 34.18834198 | 20.6039679 |
| AA488084 | 14.20448178 | 13.42950914 | 12.7036246 | 18.9618 | 14.49387186 | 17.34183829 | 18.41774811 | 10.91028856 |
| R89715 | 19.00984517 | 16.78226171 | 15.93191526 | 23.59033847 | 19.8478327 | 20.09882291 | 20.88101303 | 13.98761153 |
| AA490501 | 17.29764034 | 18.48394335 | 14.52922019 | 15.77576344 | 14.10802818 | 23.00662243 | 25.37702488 | 14.90184018 |
| N32199 | 27.83247395 | 27.16433957 | 27.83599929 | 17.63576421 | 20.01897529 | 29.53277745 | 35.16676364 | 20.63729373 |
| AA434404 | 12.98452113 | 16.11788081 | 13.26212785 | 17.45606094 | 16.25005553 | 12.59970441 | 11.14968041 | 6.811542045 |
| N93688 | 28.28483481 | 25.54412177 | 19.80975005 | 17.76258329 | 17.42508421 | 22.88245448 | 31.60912628 | 17.69865454 |
| AA292676 | 9.876235182 | 8.317319028 | 3.210728167 | 4.436837751 | 4.953957507 | 9.622367291 | 7.40323057 | 7.63997429 |
| AA464417 | 11.05434122 | 11.16528302 | 7.715140755 | 4.628342903 | 7.257940789 | 14.12507957 | 12.84862626 | 10.51986876 |
| AA442092 | 5.48651812 | 6.31065156 | 4.641902691 | 4.599175502 | 3.885904241 | 4.692019402 | 4.440172458 | 3.114766105 |
| AA442092 | 5.48651812 | 6.31065156 | 4.641902691 | 4.599175502 | 3.885904241 | 4.692019402 | 4.440172458 | 3.114766105 |
| AA026644 | 0.62252822 | 0.701896284 | 0.261093119 | 4.191592598 | 6.49E−02 | 0.029204697 | 3.36E−02 | 0.437058833 |
| AA481464 | 18.63468043 | 20.94766373 | 14.27769307 | 15.75348631 | 11.46364273 | 12.65501458 | 9.009332352 | 10.36580755 |
| T68859 | 19.81384726 | 21.31173502 | 19.75635729 | 20.39980475 | 20.05062771 | 19.46428757 | 19.33000277 | 13.65552685 |
| AA699560 | 20.82023648 | 16.7911717 | 18.43819663 | 25.52023654 | 19.85201903 | 17.12500341 | 12.21472729 | 10.2413573 |
| AA705069 | 20.88685524 | 23.16148711 | 15.0840977 | 20.67586612 | 14.18817428 | 19.01580429 | 13.44540219 | 13.6412039 |
| AA457739 | 33.37327991 | 35.6883041 | 34.4823112 | 20.55802853 | 31.64214174 | 37.33300573 | 37.02468042 | 24.08688689 |
| H99843 | 16.79713092 | 16.69352615 | 7.127333563 | 18.73583293 | 9.995156003 | 14.60344228 | 16.5049794 | 12.29468204 |
| AA399410 | 29.72998467 | 33.11698129 | 23.43060422 | 25.72188937 | 29.04178273 | 34.63630675 | 33.29299911 | 21.29525921 |
| AA443039 | 17.93908822 | 18.28745065 | 29.34817189 | 13.16317634 | 17.21193969 | 18.81890352 | 17.62059907 | 13.67676164 |
| AA164440 | 9.30576802 | 8.984327957 | 9.868215304 | 8.433442763 | 8.752113579 | 8.431813452 | 5.330326944 | 5.313034392 |
| AA446453 | 12.86153172 | 14.28408559 | 6.56088494 | 7.913354462 | 9.798358874 | 11.52903302 | 10.6491586 | 9.31631564 |
| AA280692 | 13.47620777 | 13.91271582 | 10.59679067 | 8.217327335 | 12.09365638 | 12.02460496 | 17.61991673 | 10.08089846 |
| AA031514 | 12.90000518 | 15.02943076 | 7.652758135 | 8.941687918 | 10.4641265 | 13.93556692 | 15.68826294 | 11.63565104 |
| R33154 | 15.95001951 | 18.20484579 | 10.1215106 | 7.276840559 | 13.99489447 | 13.57306878 | 14.10166919 | 10.83686664 |
| AA487452 | 8.187976148 | 8.458602154 | 9.876629011 | 5.590565736 | 5.757265048 | 9.281445194 | 7.000673633 | 5.618451692 |
| AA400329 | 18.57548571 | 17.4980898 | 9.303751927 | 13.38561433 | 13.86149745 | 18.50617543 | 19.11656532 | 13.89116259 |
| AA454668 | 25.89304171 | 31.51348528 | 22.93446371 | 22.57583786 | 26.88624881 | 28.81055744 | 19.75055767 | 14.91854781 |
| AA486393 | 0.182124982 | 0.615575177 | 4.602814218 | 1.268031115 | 0.231530443 | 0.120506359 | 0.294243975 | 1.047822638 |
| R62541 | 6.08E−02 | 8.40E−02 | 0.282420426 | 1.89326344 | 0.22723694 | 0.55787894 | 0.265300367 | 0.465287427 |
| AA171613 | 0.27466036 | 1.123906401 | 5.619710529 | 1.64076918 | 0.206048231 | 0.318509847 | 0.213712273 | 0.784934532 |
| AA235706 | 57.38142594 | 60.53251272 | 55.52482329 | 61.56762824 | 83.23998616 | 63.03392579 | 63.61974345 | 26.90748839 |
| AA668527 | 0.3985816 | 0.597438821 | 4.966520787 | 1.47871208 | 0.198743181 | 0.088338506 | 5.14E−02 | 1.584106321 |
| T64144 | 9.38E−02 | 0.192484503 | 1.850866515 | 0.17162783 | 6.49E−02 | 4.08E−02 | 0.166840739 | 0.696340115 |
| R14080 | 9.55E−02 | 0.107642909 | 1.600491683 | 0.379619373 | 0.14791746 | 9.50E−02 | 0.055410162 | 0.820779184 |
| AA609599 | 0.187186514 | 0.10012971 | 0.27386765 | 1.420850931 | 0.186769847 | 0.275819631 | 0.221567336 | 0.689208446 |
| AA489201 | 0.14443693 | 0.214598987 | 2.006534993 | 0.32146219 | 9.07E−02 | 4.93E−02 | 0.063098942 | 0.80944825 |
| R08876 | 0.568537573 | 0.970149717 | 1.576770394 | 0.384565828 | 0.43616572 | 0.422480014 | 0.296461364 | 1.181968079 |
| H46425 | 0.12265004 | 7.01E−02 | 6.84E−02 | 1.742654009 | 0.150967538 | 0.162468773 | 0.155852088 | 0.251147304 |
| R56149 | 0.139454424 | 0.213035184 | 0.815295152 | 0.482793426 | 0.717693865 | 0.297475848 | 3.81E−02 | 2.113668202 |
| AA454619 | 3.288336383 | 3.22099397 | 1.915196108 | 1.919736037 | 2.762560824 | 3.92242617 | 3.716888609 | 5.217509513 |
| H15445 | 0.120219632 | 0.111757383 | −3.02E−02 | 0.467435552 | 4.96E−02 | 1.52E−02 | 9.01E−02 | 0.41450502 |
| AA705225 | 0.18852753 | 0.180935638 | 0.94755028 | 0.256955531 | 0.098583152 | 4.21E−02 | 2.79E−02 | 0.674486801 |
| AA191488 | 0.8106325 | 1.339071481 | 9.778098245 | 9.71763194 | 0.472492022 | 0.577988082 | 0.368988511 | 1.716820958 |
| N64862 | 0.177514818 | 0.134938261 | 0.724810552 | 0.540552309 | 0.142613803 | 8.88E−02 | 8.34E−02 | 1.380235208 |
| R45413 | 0.121785119 | 0.177364842 | 0.66590467 | 2.22765821 | 8.94E−02 | 0.189579838 | 0.126874076 | 0.639711527 |
| R77293 | 17.32761828 | 18.58361324 | 4.930581246 | 11.25376919 | 13.83440333 | 12.64389145 | 12.77350206 | 10.14808689 |
| AA436187 | 15.01390271 | 13.59205508 | 9.447023349 | 23.31188756 | 9.068747717 | 12.4718263 | 13.46398542 | 8.219025894 |
| AA676470 | 30.08495758 | 24.12483149 | 26.19627013 | 36.68374593 | 38.36383944 | 30.07336868 | 29.97197759 | 18.93190315 |
| AA443634 | 8.40E−02 | 0.248574668 | 1.87808441 | 0.506000681 | 0.115582035 | 2.84E−02 | 1.86E−02 | 0.530955426 |
| AA664180 | 36.06326367 | 31.7886287 | 31.69253658 | 48.48811822 | 47.96493814 | 35.23111673 | 43.94952253 | 18.50826673 |
| W58658 | 0.173902417 | 0.15340018 | 0.620011724 | 1.529093442 | 0.023894127 | 1.68E−02 | 1.45E−02 | 0.702231009 |
| H54023 | 0.247467714 | 0.188125663 | 0.227984638 | 5.912011779 | 0.223991418 | 0.399206145 | 0.253395926 | 1.386292307 |
| H73724 | 0.088814629 | 8.03E−02 | 0.231928251 | 1.683504664 | 0.117575022 | 0.155606877 | 0.129032488 | 0.444812754 |
| T70031 | 0.277245911 | 0.148769574 | 7.22E−02 | 6.834546017 | 0.22727827 | 7.26E−02 | 4.66E−02 | 4.39E−02 |
| AA481758 | 69.05370315 | 37.39817347 | 42.70517496 | 58.7187498 | 64.37630302 | 40.66376745 | 37.7884949 | 23.04551033 |
| AA521431 | 61.83605618 | 43.27436449 | 55.26120705 | 77.90743727 | 76.13351238 | 40.95637623 | 42.00937518 | 25.13021963 |
| AA446103 | 0.104636512 | 7.03E−02 | 7.95E−02 | 1.215881577 | 9.41E−02 | 0.149593049 | 0.125073625 | 0.646399796 |
| N92646 | 28.06784683 | 30.96204311 | 27.96134068 | 36.82918822 | 30.95508803 | 36.04823224 | 35.37693112 | 18.89015759 |
| AA453789 | 1.808404003 | 2.62249449 | 2.812909827 | 1.746220767 | 2.577090448 | 1.40223369 | 1.246611512 | 3.36017901 |
| AA425299 | 13.08032469 | 13.71772041 | 14.28834182 | 16.54671153 | 17.41154591 | 18.68745962 | 13.52405931 | 12.63625711 |
| AA868929 | 0.618942483 | 0.614412924 | 0.22361388 | 0.834682507 | 0.635222734 | 0.370649638 | 0.37352773 | 1.258217182 |
| R60019 | 0.119841725 | 0.116811729 | 0.259384551 | 3.00499051 | 0.182169515 | 0.151089307 | 0.155852498 | 0.941525731 |
| AA857343 | 1.901017437 | 2.489976812 | 0.714479672 | 1.232686475 | 0.932256949 | 1.974939699 | 1.554741816 | 1.884138006 |
| AA481438 | 1.201789068 | 1.353488237 | 0.871369975 | 1.070764965 | 0.757366565 | 0.88003401 | 0.80663823 | 2.827484886 |
| AA399674 | 1.105347969 | 1.501678874 | 0.866552209 | 0.74069806 | 0.55317612 | 0.938530357 | 0.733003255 | 1.646361253 |
| T98887 | 0.124035858 | 0.312987764 | 2.343639547 | 0.401768082 | 0.207652428 | 0.506081312 | 0.158684645 | 0.746649314 |
| AA678404 | 2.36841611 | 3.938382636 | 1.58773702 | 2.194204209 | 2.061478452 | 3.607585825 | 3.37665162 | 3.542744541 |
| H15747 | 0.287743257 | 0.229134462 | 0.372656688 | 1.777298932 | 4.66E−02 | 5.65E−02 | 0.041067986 | 1.289613816 |
| H16958 | 1.894931477 | 3.000533791 | | 8.633043094 | 3.547286301 | 1.619498817 | 5.733681715 | 1.283158823 |
| AA936783 | 0.389167344 | 1.499581511 | 10.64077637 | 2.384094632 | 0.13911189 | 0.66026872 | 0.277728232 | 1.169970181 |
| AA884709 | 2.198015928 | 2.066757874 | 1.718800124 | 1.980966588 | 2.355838772 | 0.830886735 | 0.335287796 | 4.749490887 |
| H24688 | 0.201982284 | 0.219962769 | 0.751879444 | 0.512830988 | 0.153838277 | 0.734814109 | 7.99E−02 | 1.552613702 |
| AA884403 | 2.103460786 | 2.672782071 | 1.300637897 | 1.099433595 | 1.733827895 | 1.274946177 | 1.242053454 | 2.973138744 |
| AA404619 | 0.15138684 | 0.357518214 | 3.52385316 | 0.69841884 | 0.179573378 | 0.18311414 | 0.182224032 | 0.75943341 |
| AA598611 | 0.330074441 | 0.226908429 | 1.350802268 | 1.655404779 | 0.200113403 | 0.186066173 | 0.124232766 | 1.678785386 |
| H72875 | 0.179741117 | 0.13827264 | 0.473352965 | 0.490771985 | 7.61E−02 | 4.42E−02 | 4.34E−02 | 1.006169064 |
| H63361 | 0.163571849 | 0.12317033 | 0.475111201 | 1.012711843 | 0.119954697 | 0.119944558 | 0.113665578 | 0.624304289 |
| R39221 | 0.186857808 | 0.316450834 | 0.432784395 | 2.516001904 | 0.671600175 | 0.711850096 | 9.23E−02 | 1.36227562 |
| R02348 | 6.20E−02 | 0.206438723 | 2.150640832 | 0.255419744 | 3.89E−02 | 2.30E−02 | 3.21E−02 | 1.140843608 |
| R51835 | 3.03E−02 | 0.363378683 | 0.731237551 | 0.726185228 | 1.322490099 | 1.631249806 | 0.224518258 | 6.856746548 |
| R33031 | 9.98E−02 | 0.162983983 | 1.827344826 | 0.62547274 | 0.062236534 | 3.12E−02 | 7.42E−02 | 0.817377295 |
| AA412053 | 0.204788914 | 0.413840453 | 1.348709648 | 0.357745167 | 1.241913181 | 0.757293992 | 0.22139368 | 1.438145557 |
| AA001897 | 0.348034638 | 0.171387974 | 0.207561282 | 1.765380433 | 0.543185967 | 0.658838523 | 6.58E−02 | 0.748430368 |
| W81191 | 1.790574222 | 1.899247237 | | 1.807214492 | 0.346308978 | 0.222762323 | 0.107712128 | 1.34541522 |
| AA430552 | 2.91607572 | 3.076041003 | 2.829597507 | 4.426370714 | 4.729925458 | 4.71187164 | 4.531847561 | 3.70805471 |
| AA594130 | 0.703542309 | 1.005837145 | 0.525583509 | 1.216578849 | 0.691717084 | 0.376548847 | 0.27857931 | 0.635247713 |
| N92864 | 0.079866033 | 0.112472409 | 0.251854929 | 0.452282211 | 0.101581389 | 0.227029764 | 0.237170156 | 0.466949248 |
| AA457123 | 0.216922531 | 0.155819343 | 0.364362183 | 0.723388363 | 0.133198078 | 7.45E−02 | 0.10617339 | 0.52975191 |
| R43320 | 9.13E−02 | 7.59E−02 | 0.458730796 | 2.051917813 | 0.104074549 | 0.137491428 | 0.108125471 | 1.014817239 |
| AA670430 | 9.678888892 | 6.883278692 | 5.717565814 | 7.613099058 | 8.65181804 | 7.995091885 | 4.8996271 | 5.249095284 |
| H85068 | 0.34278548 | 0.256922183 | 0.586815168 | 4.298907537 | 0.476155756 | 0.479705389 | 6.74E−02 | 1.020767749 |
| AA458785 | 0.785442166 | 1.506491022 | 9.808733277 | 1.828902106 | 0.920171532 | 0.658849251 | 0.698071385 | 1.994825669 |
| AA485871 | 0.597816889 | 1.768341753 | 11.84398613 | 2.137826817 | 0.262843105 | 0.254992284 | 0.126660693 | 1.303743017 |
| T39411 | 0.396231188 | 0.296618072 | 0.324548562 | 1.969945161 | 0.169337341 | 0.268104021 | 0.247950339 | 1.580361635 |
| R00855 | 1.087364016 | 1.024237951 | 0.765054603 | 0.975300349 | 0.539506121 | 0.412648188 | 0.154076211 | 0.733947191 |
| H98666 | 2.109706251 | 2.780481372 | 1.275784185 | 1.529882866 | 1.784602477 | 2.774145853 | 1.966370637 | 3.844830747 |
| H72028 | 0.181183944 | 0.425147123 | 0.857472955 | 1.001641974 | 0.163941215 | 0.967420557 | 0.351793709 | 0.907756492 |
| AA679177 | 0.348135481 | 0.500333332 | 1.326832638 | 4.439877031 | 0.32836439 | 0.421388335 | 0.341562345 | 1.848279914 |
| N21578 | 0.168101047 | 0.117805103 | 0.409669051 | 1.325818704 | 8.67E−02 | 2.22E−02 | 2.56E−02 | 0.494287318 |
| AA007419 | 0.175440269 | 0.12813069 | 0.39815608 | 0.955234641 | 0.161389912 | 0.196276298 | 0.170532566 | 1.071322887 |
| T49657 | 0.308720496 | 0.570684257 | 4.039761403 | 1.234676181 | 0.125959577 | 3.27E−02 | 1.97E−02 | 1.338939336 |
| N38959 | 7.55E−02 | 1.055602229 | 7.313895345 | 1.268404297 | 0.307559568 | 0.724991758 | 0.070393719 | 1.035781395 |
| R51912 | 0.30750311 | 0.157082387 | 0.297430002 | 3.304783027 | 0.316950074 | 0.28696647 | 0.164570106 | 1.141683339 |
| H90415 | 0.332923734 | 1.116687454 | 9.324562689 | 1.997045708 | 0.350224199 | 0.100034162 | 8.78E−02 | 1.169187709 |
| H41489 | 0.135065338 | 0.112846912 | 0.31963146 | 0.540685076 | 0.105448274 | 2.38E−02 | 2.54E−02 | 0.533589747 |
| H16456 | 0.118493184 | 8.15E−02 | 0.670523766 | 0.212363873 | 9.91E−02 | 0.048036092 | 3.66E−02 | 1.044685298 |
| W45415 | 0.187478377 | 5.20E−02 | 0.722058962 | 0.692564761 | 0.293080465 | 0.189933858 | 7.54E−02 | 0.655830442 |
| AA447751 | 0.78396734 | 0.786015622 | 0.528464235 | 1.211081391 | 0.239987152 | 0.17186979 | 0.272698223 | 1.030511383 |
| AA487486 | 0.534940441 | 0.350108402 | 1.666390776 | 0.795295661 | 0.359287561 | 0.193793075 | 0.198174164 | 1.934418861 |
| R56604 | 0.3460236 | 0.406195433 | 1.083709289 | 0.653908131 | 0.50487088 | 0.489048814 | 0.385606236 | 1.682176098 |
| T65772 | 0.25838637 | 0.106483973 | 0.235991068 | 0.28337251 | 0.46880307 | 0.109303057 | 0.116698413 | 1.388384089 |
| H15085 | 0.157580914 | 0.114768422 | 0.508468022 | 0.673401507 | 0.17757899 | 6.65E−02 | 6.08E−02 | 0.136004744 |
| R61295 | 0.212944574 | 0.159174688 | 1.555313951 | 0.607244235 | 0.169955202 | 0.225692165 | 0.172935079 | 0.384976032 |
| T61256 | 7.25E−02 | 0.151774275 | 0.761713647 | 0.145248528 | 8.64E−02 | 7.32E−02 | 6.14E−02 | 1.055956615 |
| AA405731 | 0.195114794 | 0.136469531 | 0.339111129 | 3.35219508 | 0.125085325 | 0.189941763 | 0.145965169 | 0.612889025 |
| T71879 | 0.128933321 | 0.137341443 | 0.390507256 | 0.407085926 | 0.114225823 | 4.49E−02 | 2.40E−02 | 0.521752069 |
| R59927 | 9.10E−02 | 0.107392035 | 8.50E−02 | 0.442229621 | 9.27E−02 | 6.81E−02 | 9.43E−02 | 0.347354052 |
| AA496780 | 0.110593994 | 0.022497663 | 0.534503826 | 1.752767599 | 0.281792243 | 0.415100922 | 8.75E−02 | 1.131163315 |
| AA176888 | 0.178362771 | 0.144369523 | 0.781888663 | 1.331812939 | 0.187224487 | 9.28E−02 | 6.82E−02 | 0.650312155 |
| AA436163 | 0.23227633 | 0.236489348 | 0.254658187 | 2.416941823 | 0.161958036 | 0.3988222 | 0.305725744 | 1.256999654 |
| AA428778 | 0.105802992 | 9.28E−02 | 0.486961912 | 2.193301764 | 0.163889027 | 0.132381001 | 7.25E−02 | 0.611181122 |
| AA463225 | 0.276223908 | 0.152997555 | 1.039475243 | 2.155030865 | 0.418387856 | 0.329192564 | 0.19424438 | 1.916291602 |
| AA485426 | 0.289852714 | 0.373831514 | 0.418062891 | 1.661826021 | 0.22603134 | 0.194563224 | 0.121355588 | 1.266933317 |
| W47485 | 0.290711332 | 0.28058818 | 0.290834133 | 1.405085787 | 0.21336519 | 0.172813844 | 2.02E−02 | 0.962779151 |
| H84982 | 0.131639592 | 0.139838613 | 0.317575218 | 2.016302257 | 9.62E−02 | 0.376525133 | 9.28E−02 | 0.503147875 |
| AA504615 | 0.260955437 | 0.211851667 | 0.911938546 | 0.668568083 | 0.134885637 | 0.121344263 | 0.121161862 | 1.972320295 |
| H94487 | 0.176887945 | 0.365682094 | 0.630918749 | 2.620578978 | 0.183460578 | 0.419558641 | 0.179194474 | 1.679828681 |
| AA448959 | 2.916692897 | 2.932033692 | 1.637325243 | 13.34753362 | 0.628009453 | 0.180111066 | 0.132777658 | 2.07701127 |
| AA070358 | 4.54E−02 | 4.12E−02 | 0.367054172 | 0.116479427 | 4.89E−02 | 2.03E−02 | 2.61E−02 | 0.244403144 |
| AA453401 | 0.167660345 | 0.214972768 | 0.222252985 | 0.203904483 | 0.242997298 | 0.251569524 | 0.177121661 | 0.804209416 |
| N66737 | 4.20E−02 | 7.41E−02 | 0.257268707 | 26.36051857 | 1.048190646 | 0.091475566 | 0.104092386 | 0.79476014 |
| AA666180 | 3.196631019 | 7.187377922 | 29.76323485 | 10.64207727 | 2.761542964 | 3.990641982 | 4.044434382 | 4.868184378 |
| AA857131 | 0.223573307 | 9.63E−02 | 0.758187241 | 23.03526077 | 0.070686238 | 3.60E−02 | 4.56E−02 | 0.878719343 |
| AA479102 | 6.852949035 | 8.120715713 | 3.236793772 | 11.51815786 | 4.73539654 | 7.003983588 | 6.605548634 | 5.888262807 |
| AA456077 | 1.027879631 | 1.325946518 | 1.245447159 | 2.318900816 | 1.481005766 | 0.592776193 | 0.435487259 | 1.471236654 |
| R87497 | 0.319714784 | 0.441278788 | 1.241443943 | 2.794768846 | 5.52E−02 | 2.59E−02 | 7.17E−02 | 1.35489803 |
| AA718910 | 0.771846813 | 0.859478249 | 0.801090192 | 1.362044268 | 0.616842421 | 0.429437896 | 0.408760631 | 1.063445986 |
| AA406269 | 0.270836576 | 0.224358482 | 1.06080255 | 1.245738874 | 0.124219479 | 6.82E−02 | 9.79E−02 | 0.972478072 |
| N74623 | 0.147826314 | 0.155576422 | 0.39183835 | 7.661750863 | 0.116891312 | 4.48E−02 | 3.09E−02 | 0.687478091 |
| H99364 | 0.547513791 | 0.368774275 | 1.09104024 | 1.578071783 | 0.315822232 | 0.108583159 | 0.10216456 | 1.465520886 |
| AA447684 | 0.497110821 | 0.537175623 | 0.525424572 | 1.458911244 | 4.98E−02 | 2.74E−02 | 5.04E−02 | 0.735202872 |
| AA282301 | 0.27173204 | 6.68E−02 | 0.520517404 | 3.191486412 | 0.722232552 | 1.277477958 | 0.144496875 | 2.236318764 |
| H99588 | 0.227706892 | 0.166835375 | 0.379322092 | 7.180929201 | 0.205434573 | 0.247795568 | 0.187784708 | 1.219437291 |
| N53512 | 0.693352824 | 0.85093842 | 0.861714576 | 5.362013301 | 0.478594275 | 0.673037164 | 0.544521467 | 1.733748427 |
| AA683321 | 0.246596976 | 0.176033115 | 0.293208249 | 5.563771987 | 0.123251357 | 0.226202587 | 0.181288584 | 1.448843062 |
| AA608557 | 0.385872016 | 0.383796565 | 0.952099841 | 3.879470723 | 0.316768638 | 0.312030768 | 0.238687939 | 1.726847773 |
| AA757764 | 0.297379076 | 0.165726325 | 0.88261567 | 0.440905543 | 0.372520434 | 0.382055076 | 0.145493758 | 0.988760934 |
| AA406064 | 0.963197456 | 1.184985842 | 0.779087405 | 0.891603525 | 0.966885185 | 0.579894823 | 0.330253413 | 1.152159636 |
| N54598 | 29.7307313 | 23.60460158 | 15.15010606 | 61.1100551 | 21.20150657 | 23.73876744 | 23.56652312 | 20.42284808 |
| AA481988 | 0.83030089 | 1.67920494 | 1.521390918 | 1.602447026 | 1.594545777 | 1.393612985 | 0.586783337 | 1.454014827 |
| N62394 | 0.292745156 | 0.145445316 | 0.919547638 | 0.486071328 | 0.618336636 | 0.192065885 | 0.209279951 | 1.81964171 |
| N20148 | 0.36264957 | 0.293240313 | 1.191190173 | 0.634599551 | 0.725999262 | 0.465883026 | 1.173321753 | 2.675970931 |
| AA496678 | 0.319441345 | 0.257667574 | 0.478071395 | 0.622386812 | 0.132931879 | 1.95E−02 | 2.60E−02 | 0.840188206 |
| AA400973 | 1.534002053 | 1.714822504 | 1.436906175 | 1.048156955 | 1.254760066 | 0.941922627 | 0.722913601 | 1.95298952 |
| AA497027 | 0.438480922 | 1.508084468 | 0.372150077 | 3.020669538 | 0.359906823 | 0.227232464 | 0.14420936 | 1.043280575 |
| N64508 | 2.009112662 | 3.452207016 | 0.792805621 | 1.301784349 | 2.539009334 | 3.500903196 | 3.083422958 | 4.592914592 |
| AA033564 | 0.069421532 | 0.161663182 | 0.323575075 | 0.250293874 | 0.17400072 | 0.226600648 | 9.11E−02 | 0.871274685 |
| AA446108 | 0.769668027 | 0.390512087 | 4.097425592 | 1.207299335 | 0.354854654 | 0.436418383 | 0.134273293 | 1.258329856 |
| AA159577 | 0.446474167 | 0.379318202 | | 1.090721231 | 0.206696214 | 4.62E−02 | 3.77E−02 | 0.916706477 |
| R36958 | 5.23E−02 | 2.39E−02 | 0.262096407 | 0.139051554 | 0.101530951 | 5.47E−02 | 4.12E−02 | 0.458047702 |
| AA829508 | 0.630754179 | 1.081371373 | 11.12264238 | 0.264354582 | 0.128126994 | 0.765515506 | 1.149526797 | 3.042979924 |
| AA482067 | 0.205039081 | 0.253788068 | 0.282480027 | 0.493737706 | 0.158658574 | 7.90E−02 | 5.28E−02 | 0.55095889 |
| AA669314 | 0.683742215 | 0.808392548 | 1.16372394 | 1.602848914 | 0.442885133 | 0.570227867 | 0.280240919 | 0.712164672 |
| AA775241 | 1.418874982 | 1.846198647 | 5.577800931 | 1.344689512 | 0.880185699 | 1.252484239 | 1.424573602 | 3.384055572 |
| R73584 | 1.02446963 | 0.935468411 | 0.803404707 | 0.368215322 | 0.286257374 | 0.109893655 | 7.53E−02 | 1.275588944 |
| H28984 | 16.59350558 | 16.18200978 | 8.172410684 | 13.1191606 | 14.4503845 | 11.12950391 | 7.932771347 | 9.633157025 |
| R44202 | 8.69E−02 | 4.382−02 | 0.253533697 | 0.166629344 | 0.117904267 | 0.101127842 | 0.103847056 | 1.059451655 |
| W70051 | 21.76297737 | 20.04405194 | 14.91138327 | 16.87667976 | 19.87488409 | 28.04573416 | 30.08594829 | 16.42056168 |
| AA401972 | 14.92249068 | 16.42885435 | 5.611565027 | 8.854697045 | 13.81855051 | 16.92468552 | 18.75158154 | 15.74888433 |
| AA236164 | 20.21677625 | 18.79310438 | 12.57587929 | 11.95096823 | 16.36247541 | 20.16185377 | 20.77848532 | 15.16538035 |
| R22412 | 15.72505574 | 17.55060555 | 15.82656015 | 13.78588969 | 13.30933358 | 14.5213203 | 15.01308178 | 13.15195793 |
| AA424804 | 14.70872577 | 15.97533753 | 10.78900463 | 13.01835661 | 12.64309311 | 15.36509292 | 16.14117886 | 11.65209772 |
| AA669443 | 16.40914268 | 14.22534282 | 9.970977767 | 18.06831801 | 15.3193005 | 15.5104445 | 18.01732542 | 11.20069358 |
| N69689 | 11.40467488 | 11.65322402 | 6.372712462 | 5.538040372 | 6.533556933 | 11.1584167 | 11.60779004 | 8.597842756 |
| H24316 | 16.83164161 | 15.705615 | 15.68021927 | 15.56699579 | 19.40991358 | 20.01447688 | 19.29795097 | 0.497864329 |
| AA074224 | 0.333624301 | 0.375913694 | 0.378626744 | 3.784608939 | 0.327739524 | 0.489769841 | 0.322978295 | 1.313192294 |
| R36571 | 15.28422925 | 17.32979473 | 15.34324385 | 15.25547675 | 18.55691796 | 14.2447373 | 10.72233164 | 1.060647719 |
| AA058465 | 18.42366544 | 15.89605854 | 8.341211937 | 14.29501218 | 17.71874821 | 13.05741368 | 16.65715974 | 11.02252852 |
| AA633811 | 22.16035468 | 22.782521 | 30.6526342 | 14.91020806 | 19.44240872 | 23.66144082 | 26.37266059 | 17.24459371 |
| AA457155 | 21.68263476 | 12.69269947 | 15.02783414 | 18.54841663 | 19.13660886 | 15.98507842 | 11.33190117 | 10.09290381 |
| AA459104 | 15.2195866 | 16.20194954 | 24.24665432 | 13.86213005 | 18.26771629 | 15.71979923 | 13.29493507 | 12.57587877 |
| R40212 | 14.5790754 | 17.12092824 | 17.93072196 | 13.93477495 | 17.42255813 | 17.93870181 | 18.31265802 | 2.2735197 |
| AA086476 | 17.74782242 | 18.69369476 | 14.58097392 | 18.97637683 | 20.8754483 | 20.68533459 | 19.26034455 | 12.52733321 |
| AA683310 | 18.34330926 | 18.98378867 | 6.057610695 | 11.98270843 | 15.69453266 | 15.19045985 | 18.66892542 | 11.4721218 |
| AA455640 | 18.47809437 | 17.61623038 | 7.452925146 | 8.994012338 | 8.980063651 | 12.54788533 | 16.22279878 | 12.11311137 |
| AA496878 | 5.246470108 | 6.492383676 | 7.23111917 | 5.567148567 | 7.297448165 | 6.519473799 | 6.047516043 | 4.881896262 |
| AA085749 | 10.15319844 | 10.58867058 | 3.720477618 | 4.092990024 | 7.461499363 | 6.388572117 | 7.203726545 | 6.728320601 |
| AA425755 | 13.24455202 | 12.88357607 | 5.034714528 | 6.268355719 | 9.082441534 | 11.59487571 | 11.62952447 | 9.529595044 |
| N52350 | 10.61031686 | 10.79705267 | 12.63852019 | 8.6754759986 | 8.976553332 | 3.221438906 | 2.335184873 | 1.494699625 |
| AA630104 | 12.7227255 | 13.72066077 | 6.71563807 | 7.938528099 | 6.913277923 | 14.18776318 | 13.90026724 | 11.35565277 |
| AA454854 | 10.92967966 | 11.23143802 | 4.418447748 | 5.174772829 | 7.976384214 | 9.703784616 | 10.93778907 | 9.331503041 |
| W73406 | 7.903009948 | 8.279041624 | 2.830978933 | 2.857181738 | 4.639918805 | 4.149802613 | 3.606526999 | 4.719619102 |
| R12802 | 0.238444224 | 0.16825892 | 1.58807476 | 0.822064291 | 7.205178124 | 0.257279583 | 0.444604195 | 1.264969686 |
| AA465355 | 7.336819392 | 5.300937536 | 2.387694789 | 1.77122396 | 6.072708998 | 2.990994851 | 3.612925236 | 6.445676951 |
| AA829383 | 9.713113247 | 10.66886848 | 10.54302042 | 11.63031161 | 8.843010597 | 9.775032469 | 9.586544135 | 5.681120215 |
| AA629189 | 11.36797877 | 14.967791 | 4.768128161 | 7.170347339 | 9.57906376 | 11.61769675 | 10.65243226 | 8.340390966 |
| AA430512 | 9.534573517 | 8.247953524 | 8.316348291 | 9.290581043 | 11.64863395 | 8.113650285 | 5.996962103 | 6.214691332 |
| AA456439 | 10.70832344 | 10.50311685 | 10.50165717 | 9.291870768 | 11.97285965 | 10.03817061 | 8.954783916 | 6.730899031 |
| H27864 | 9.780274543 | 10.17961553 | 7.795175273 | 8.32114728 | 10.79728458 | 9.655471293 | 7.995744552 | 7.171240518 |
| AA644657 | 14.97773748 | 14.72866784 | 15.19497586 | 17.4722782 | 14.48792372 | 13.4943961 | 14.66848169 | 8.981574288 |
| R40460 | 14.02006989 | 13.67189221 | 6.32559769 | 8.741499108 | 13.22962391 | 14.28706841 | 14.74331878 | 0.86098332 |
| W96058 | 8.316574942 | 9.202396521 | 4.185436741 | 5.802080978 | 9.090198422 | 6.944350085 | 7.898182283 | 6.501582684 |
| T72202 | 4.910901674 | 5.633601899 | 5.685609621 | 4.651309743 | 4.997212679 | 7.505442874 | 5.542126135 | 4.689359807 |
| AA598794 | 0.573341181 | 8.49E−02 | 1.038303088 | 0.244900677 | 0.310786175 | 7.77E−02 | 0.048832125 | 0.163149057 |
| AA599178 | 0.570691633 | 0.339390237 | 8.557633935 | 0.480744514 | 0.435231923 | 0.218741059 | 0.216046798 | 0.435225613 |
| R88247 | 0.532639965 | 0.545118994 | 0.620081258 | 3.141140933 | 0.150055455 | 2.45E−02 | 1.85E−02 | 0.7022889 |
| T98812 | 0.343184973 | 0.247595029 | 1.531771465 | 0.291419246 | 0.128461144 | 0.340974608 | 0.198086927 | 0.425481979 |
| AA454858 | 0.223635364 | 0.268810127 | 0.8147468807 | 0.353760373 | 0.103986984 | 3.73E−02 | 3.16E−02 | 0.245535885 |
| N67048 | 10.14341863 | 7.592113899 | 4.344522356 | 3.851620223 | 4.536042504 | 6.665109387 | 5.008133377 | 7.16695928 |
| AA776875 | 8.06934578 | 7.7581352 | 3.091038965 | 3.741685834 | 5.546996168 | 8.791269951 | 7.982378371 | 8.365452414 |
| H51117 | 3.966389223 | 5.346765011 | 1.395463454 | 2.49073858 | 3.687011927 | 3.718900078 | 3.084623398 | 3.524676899 |
| N36174 | 14.483682 | 13.54683346 | 11.11466575 | 11.63388016 | 14.05262104 | 12.75178351 | 11.89006855 | 9.330359142 |
| AA777187 | 10.42303629 | 9.748871101 | 3.642022531 | 3.229088085 | 5.366152041 | 5.862550486 | 5.823237791 | 8.241060625 |
| R09581 | 22.00681959 | 21.11752477 | 18.04191799 | 22.91566491 | 22.79559771 | 14.44791388 | 11.45011661 | 12.16467252 |
| R16849 | 8.612396808 | 7.596990399 | 4.924342984 | 3.325162711 | 4.110023154 | 7.765585109 | 7.820823897 | 5.815135246 |
| AA884167 | 4.188050396 | 4.749402103 | 1.61148811 | 2.749913448 | 4.2780267 | 4.100967576 | 3.815787701 | 4.055039882 |
| AA136983 | 9.49585425 | 10.15056984 | 3.440351838 | 4.615880778 | 6.054186478 | 6.83657547 | 7.050636979 | 8.269159968 |
| AA488622 | 9.890487467 | 9.836219264 | 3.473509988 | 3.789767104 | 6.282000648 | 8.049767976 | 6.973213882 | 8.212583544 |
| AA699427 | 7.117192529 | 7.111662492 | 2.192838503 | 3.551786441 | 5.580591133 | 4.198944242 | 5.074757452 | 4.209708436 |
| AA490459 | 2.575656166 | 3.736948711 | 0.933603593 | 1.465812184 | 1.758713261 | 1.104333244 | 0.867383854 | 1.531572668 |
| AA626787 | 2.881272998 | 4.299097477 | 2.721322605 | 1.916838494 | 4.469467633 | 5.38359605 | 5.46846531 | 6.322351973 |
| N62179 | 11.37102878 | 9.568317921 | 9.455248318 | 13.56398276 | 10.81112759 | 10.64826613 | 8.56567794 | 6.723636949 |
| N27190 | 29.54707234 | 26.25035232 | 31.35363406 | 32.67661467 | 37.57564217 | 31.43271623 | 31.53461788 | 20.77058355 |
| AA441895 | 24.27150375 | 22.9626093 | 26.60631666 | 30.86801888 | 40.06670353 | 29.78805939 | 34.54349576 | 17.24254068 |
| AA463924 | 33.402247 | 33.89347601 | 35.571325 | 30.50800393 | 34.84762552 | 32.77696512 | 34.17207689 | 20.5248861 |
| N78843 | 38.02091306 | 31.39298491 | 28.23461359 | 34.41711929 | 39.51187698 | 30.11933147 | 26.82888212 | 19.43620727 |
| AA629719 | 31.48764197 | 37.41727747 | 31.54660297 | 39.80567516 | 40.1563207 | 39.38844138 | 30.25084933 | 26.0742679 |
| AA404755 | 21.41973277 | 21.24106888 | 18.96936278 | 32.4046278 | 34.53593287 | 19.71196965 | 18.51583687 | 14.25627129 |
| AA459351 | 1.833795538 | 2.618617925 | 1.373480533 | 1.256440944 | 1.67228768 | 2.914913803 | 2.591977948 | 3.899562773 |
| AA488346 | 39.32983741 | 30.32593937 | 33.03970317 | 45.78348359 | 38.26709796 | 28.22982528 | 30.16584203 | 18.03374987 |
| AA427899 | 26.67604716 | 25.78523385 | 30.14609328 | 28.85287545 | 35.98104596 | 35.22864254 | 36.82338782 | 23.94024054 |
| AA453813 | 7.204336211 | 7.303817317 | 4.900254154 | 4.379972352 | 6.206941047 | 8.824169559 | 9.98471585 | 7.351059946 |
| AA397824 | 10.76945162 | 7.982251483 | 7.580720116 | 7.432531008 | 9.754354143 | 9.514915642 | 7.935720221 | 5.920549076 |
| AA633901 | 12.18200405 | 10.85308114 | 13.61633796 | 14.24924715 | 11.46202942 | 11.95459476 | 10.74415658 | 7.891587608 |
| AA181334 | 9.69993387 | 10.26903792 | 7.998753206 | 7.736489528 | 8.536351201 | 10.14387035 | 10.00804431 | 6.84688368 |
| AA292410 | 18.41498084 | 21.39567723 | 7.43645534 | 9.83113847 | 10.29990367 | 18.49023492 | 19.65621623 | 13.96412248 |
| AA253434 | 13.4092855 | 14.33475919 | 8.039460846 | 8.613564071 | 7.929641139 | 18.67319453 | 20.17449968 | 12.27802285 |
| AA455058 | 20.82871509 | 29.27828702 | 14.29348739 | 29.3258109 | 37.85290764 | 27.56973385 | 34.99886458 | 17.91174019 |
| R55188 | 53.79096202 | 48.27594603 | 54.21416245 | 67.09649695 | 76.87450563 | 52.63193582 | 55.51472182 | 23.62696499 |
| AA465723 | 23.38181778 | 23.20566188 | 14.35096285 | 16.08639335 | 12.51504107 | 18.1389079 | 19.37029588 | 13.58503588 |
| N49856 | 29.96970494 | 27.03519888 | 26.29856571 | 32.89736617 | 28.41338729 | 33.76040637 | 41.23242017 | 23.20296225 |
| AA455272 | 23.70820419 | 24.87271063 | 17.74038543 | 15.81791403 | 21.42687241 | 25.95981278 | 30.31298784 | 20.80534628 |
| AA459292 | 25.08753086 | 29.85942844 | 24.33982097 | 34.38597901 | 33.16899531 | 29.38762558 | 29.07865921 | 16.30430876 |
| AA878561 | 29.70997223 | 38.1181983 | 42.41594013 | 29.19560183 | 37.16368231 | 23.80169892 | 29.15914586 | 1.513482669 |
| AA772068 | 43.30930177 | 39.39007192 | 42.20898479 | 60.0670886 | 41.1840771 | 50.78509048 | 59.03512613 | 28.26604168 |
| N78821 | 31.19009514 | 31.82358739 | 34.25368088 | 37.21129767 | 43.16878807 | 28.5580241 | 29.24827598 | 16.3537237 |
| AA291490 | 45.77501651 | 46.63476256 | 53.21442136 | 56.17239995 | 68.90492839 | 54.65064586 | 57.58008452 | 35.76767897 |
| N46828 | 34.89731064 | 29.39427889 | 34.05249693 | 29.87756141 | 39.01685965 | 36.99198256 | 36.92808227 | 22.17775304 |
| AA150487 | 45.34684386 | 44.65368856 | 65.30284844 | 57.55943844 | 67.07416778 | 40.42215172 | 41.79371663 | 27.72434403 |
| AA282537 | 52.22535751 | 41.56388709 | 35.17403314 | 55.2750426 | 63.04486445 | 34.94861701 | 35.66047764 | 23.4861499 |
| AA707922 | 55.46799902 | 54.73165612 | 52.18006189 | 74.92853541 | 77.27931598 | 44.41763762 | 48.05962465 | 28.42806546 |
| AA443638 | 47.23582949 | 41.25447374 | 31.90537257 | 20.13909587 | 55.94493635 | 32.20561704 | 43.85324507 | 23.67050394 |
| W73892 | 20.27131765 | 22.07700187 | 17.82340994 | 12.42664249 | 13.03977036 | 19.26896493 | 19.76283359 | 12.52223224 |
| N70734 | 16.00676831 | 16.21613077 | 13.02268984 | 13.77803964 | 9.864935146 | 16.31280635 | 19.12043735 | 10.93049868 |
| H57136 | 29.82885337 | 23.14775485 | 17.64717904 | 22.5131702 | 27.82013988 | 19.25424118 | 19.119368361 | 16.68407949 |
| AA700414 | 29.78740203 | 29.71153223 | 30.84738122 | 35.51331266 | 28.5320194 | 35.02888476 | 37.830234117 | 23.51879387 |
| W65461 | 1.582316414 | 2.170422952 | 1.103228692 | 1.038420135 | 1.171524513 | 1.304498683 | 1.310369445 | 2.223501134 |
| AAA36584 | 9.362091141 | 8.134132036 | 6.780573609 | 9.12471574 | 10.01008974 | 10.0915921 | 7.199616982 | 5.462605703 |
| AA029042 | 30.76692002 | 23.54804667 | 16.01970787 | 20.5027043 | 24.29216348 | 19.81870818 | 18.97220606 | 14.02066164 |
| AA427725 | 24.1831519 | 26.60251326 | 23.95557184 | 27.87062775 | 19.53245951 | 29.94953555 | 31.50513283 | 19.2987866 |
| N51280 | 27.55225851 | 31.81602093 | 10.9862251 | 15.87844486 | 22.5718942 | 31.13378929 | 33.56546319 | 0.278258082 |
| AA281347 | 22.81574164 | 25.60885154 | 14.8674075 | 16.0871738 | 26.21817206 | 31.27308752 | 33.3028934 | 22.75939773 |
| AA402960 | 24.41874925 | 26.11670313 | 18.18713838 | 19.51273682 | 24.58402722 | 27.14137043 | 33.29284839 | 22.8496167 |
| N98485 | 23.15377588 | 21.24725903 | 20.03077128 | 16.99045362 | 21.34886081 | 15.26211932 | 16.22677894 | 11.5187422 |
| AA490209 | 3.035698537 | 2.915638696 | 1.659834641 | 1.10758798 | 2.258178208 | 2.051133108 | 1.835282947 | 3.040695852 |
| W61361 | 34.10379475 | 31.17761956 | 34.01922951 | 29.43169864 | 23.02878278 | 44.28043077 | 49.3716344 | 30.55831089 |
| N51018 | 0.895969739 | 0.822603424 | 8.791300555 | 6.566440211 | 0.387721776 | 0.485854727 | 0.500500102 | 3.973103926 |
| AA455281 | 3.982933258 | 5.842616596 | 3.409051258 | 2.155743141 | 3.214957653 | 5.761751821 | 6.1678428 | 6.595859335 |
| W69471 | 2.176145649 | 2.262971009 | 1.889011302 | 4.298069671 | 0.125110544 | 3.91E−02 | 4.41E−02 | 0.885776492 |
| AA486321 | 1.034914616 | 1.422616774 | 15.19920754 | 7.415723464 | 1.525470041 | 1.404546396 | 1.733369861 | 4.163095485 |
| AA458982 | 5.030129846 | 6.671175844 | 1.456266641 | 1.68375405 | 2.379536052 | 4.179216222 | 2.976132273 | 4.062391389 |
| AA442095 | 4.829174413 | 4.615478182 | 3.252727167 | 3.668199484 | 2.808813532 | 4.180626657 | 3.959090691 | 3.356087801 |
| N99003 | 2.039062485 | 2.563557284 | 1.92061982 | 1.456785844 | 2.0821831 | 1.985365512 | 1.595312179 | 1.552308165 |
| AA609284 | 2.220425329 | 2.885924256 | 1.15451555 | 1.264654536 | 2.17588501 | 1.856888897 | 1.575172599 | 3.007772428 |
| AA195036 | 31.39371467 | 32.51612495 | 19.8134058 | 22.2287943 | 29.07492796 | 24.93265482 | 25.96878567 | 17.40551832 |
| AA478268 | 11.96015041 | 10.99922076 | 11.69181426 | 14.11437595 | 16.20593802 | 9.111161437 | 8.678545684 | 6.90313221 |
| AA608583 | 30.64392285 | 26.66604793 | 25.63350599 | 30.51834358 | 30.33697486 | 24.55752432 | 28.90088593 | 20.25532836 |
| AA480435 | 21.82366631 | 20.06886964 | 18.88262313 | 22.61456267 | 25.53193168 | 21.93073616 | 21.58624772 | 13.12039825 |
| AA505046 | 23.63660096 | 22.05043745 | 23.18398426 | 18.65335792 | 26.21785542 | 23.90922453 | 31.7331025 | 17.36904023 |
| AA487893 | 20.27570383 | 19.61886578 | 17.2329505 | 24.0071361 | 28.53431361 | 17.76212522 | 25.91589986 | 13.82120963 |
| AA292226 | 48.60827655 | 42.38607553 | 23.18196775 | 29.16377551 | 30.35228689 | 41.74942514 | 54.37468138 | 30.13431188 |
| H87106 | 20.63861132 | 19.57872959 | 14.95930266 | 17.92470933 | 21.26931436 | 23.96829793 | 22.78223035 | 16.78769736 |
| W96450 | 23.07654727 | 21.55396988 | 24.36095954 | 28.19647166 | 31.37132599 | 30.73388527 | 31.10677394 | 21.79020976 |
| N33331 | 28.36940167 | 25.78662125 | 25.65907432 | 25.90328811 | 29.92364688 | 26.88595169 | 28.03401198 | 23.03958218 |
| AA405800 | 25.43365191 | 26.8056176 | 19.34783059 | 23.9119729 | 29.14739213 | 20.1376127 | 20.7481208 | 15.45920589 |
| T51539 | 37.04254749 | 35.58329862 | 27.22357679 | 29.39443068 | 29.26747712 | 32.85179948 | 37.13241221 | 24.37523886 |
| N59764 | 24.30305591 | 21.24727999 | 22.53923803 | 16.07436069 | 26.90427658 | 20.92597239 | 23.22437469 | 16.44600319 |
| AA521346 | 3.927462453 | 4.483573541 | 4.871009817 | 5.009623854 | 4.220279816 | 6.412726238 | 6.63139433 | 4.36497316 |
| AA428551 | 7.511216326 | 7.054388812 | 6.360096869 | 6.367864337 | 4.74719124 | 11.79753397 | 11.90853014 | 8.624379722 |
| AA489383 | 3.254207402 | 4.363727468 | 3.602198976 | 2.857452654 | 3.681140068 | 6.505644423 | 5.294661541 | 0.416874791 |
| AA490172 | 8.563846144 | 2.818134846 | 16.5090439 | 6.336351989 | 4.552424226 | 5.084900046 | 1.540288648 | 5.753606895 |
| AA504477 | 20.27058266 | 23.61171293 | 22.8431745 | 20.63453263 | 28.05090535 | 20.61037432 | 19.19136064 | 15.10292793 |

1-29. (canceled)

30. A method for the identification of tissue/cell specific marker genes comprising
a) taking tissue and/or cells of at least one developmental stage and/or at least one disease state, and/or
cultivating said tissue and/or cells in vitro under at least one culture condition,
b) determination of gene expression profiles of said tissue/cells and/or in vitro cultivated tissue/cells and
c) identification of specific marker genes by bioinformatic analysis of said gene expression profiles.

31. The method of claim 30, comprising cultivating tissue/cells of at least two different developmental stages and/or disease states in vitro under at least two different culture conditions, determination of gene expression profiles of said in vitro cultivated tissue/cells and identification of specific marker genes by bioinformatic analysis of said gene expression profiles.

32. The method of claim 30, wherein said tissue/cells are selected from the group consisting of fetal tissue, adolescent tissue, adult tissue, healthy tissue and pathological tissue, progenitor cells like stem cells or cells derived from the same precursor lineage.

33. The method of claim 30, wherein said culture conditions are 2D and 3D in vitro cultures.

34. The method of claim 30, wherein said gene expression profiles are determined by a micro-array.

35. The method of claim 30, wherein said bioinformatic analysis is done by software analysis like e.g. SOM or cluster analysis.

36. The method of claim 30, where the tissue is cartilage.

37. A method for the quality control of tissue biopsies and/or cells/tissue cultivated/produced in vitro comprising the steps of establishing a gene expression profile of said cells or tissue, comparison of said resulting profile with profiles characteristic for a particular status or physiological potential of the examined cells/tissue and determination of the particular status of the examined tissue/cells.

38. The method of claim 37, wherein said profile is a gene expression profile which is determined by means of a micro-array.

39. The method of claim 37, wherein said tissue is cartilage tissue or chondrocytes, preferably derived from arthritic joint tissue (rheumatoid and osteoarthritis), and a micro-array comprises polynucleotide probes of tissue specific marker genes.

40. The method of claim 37, for the assessment whether the tissue biopsy/cell samples can be used for culturing cells in vitro, the cultured tissue/cells being suitable for and/or used in the treatment of cartilage defects.

41. The method of claim 37, for the assessment whether certain therapeutic approaches such as tissue engineering therapy, cell therapy or surgical therapy can be performed.

42. The method of claim 37, for the evaluation of the quality of an in vitro produced implant or of in vitro cultivated cells for cell therapy.

43. The method of claim 37, for the use in diagnosis within clinical applications and gene expression profiles used as a diagnostic tool therein, said method comprising assessing patient biopsy/cell samples for functionality, culturing functional cells in an in vitro cell culture to get cultured cells/tissue for the treatment/performance in a cell or tissue based therapy.

44. The method of claim 37, for the use in diagnosis within clinical applications and gene expression profiles used as a diagnostic tool therein, said method comprising assessing patient biopsy/cell samples and deciding on a subsequent therapeutic approach, said approach preferably being selected from the group consisting of tissue engineered therapy, cell therapy, and traditional surgical approach.

45. The method of claim 37, for the use in quality control and gene expression profiles used as a quality control tool therein, said method comprising assessing the quality of human biopsy/cell samples prior to performing a cellular expansion in case of cell therapy and/or prior to performing the differentiation/tissue formation in case of a tissue engineering therapy.

46. The method of claim 37, for the use in quality control and gene expression profiles used as a quality control tool therein, said method comprising assessing the quality of the final implant prior and/or after product release, said implant being proliferated cells in case of a cell therapy or tissue engineered cartilage in case of a tissue engineered therapeutic approach.

47. A method for the determination of characteristic gene expression profiles for clinical use comprising:
a) determining gene expression profiles of tissue or cell samples in vitro and generating a database containing said gene expression profiles,
b) correlating patient datas e.g. patient history, medication etc. of the tissue or cell sample donor with the gene expression profile of said tissue or cell samples and optionally the clinical outcome after treatment.

48. The method of claim 47, wherein said gene expression profile has been determined by a method for the quality control of tissue biopsies and/or cells/tissue cultivated/produced in vitro comprising the steps of:
establishing a gene expression profile of said cells or tissue, comparison of said resulting profile with profiles characteristic for a particular status or physiological potential of the examined cells/tissue and determination of the particular status of the examined tissue/cells.

49. A cartilage array comprising a plurality of different polynucleotide probe spots stably associated with a solid surface of a carrier, whereby each of said spots is made of a unique polynucleotide that corresponds to one specific cartilage marker gene.

50. The cartilage array of claim 49, comprising at least two spots that have different nucleotide sequences but of the same cartilage marker gene.

51. The cartilage array of claim 49, comprising at least 10 spots of different nucleotide sequences and being indicative of a specific tissue or cell status.

52. The cartilage array of claim 49, comprising spots of different nucleotide sequences and that are indicative for at least two tissue or cell status, preferably 3 status.

53. The cartilage array of claim 49, wherein at least part of the cartilage marker genes is selected from the 467 genes listed in the description, preferably at least 10%, more preferably at least 50%, most preferably about 100%.

54. The cartilage array of claim 49, wherein said different polynucleotides of the array do not cross hybridise under stringent conditions with each other.

55. The cartilage array of claim 49, wherein the status is selected from biopsies and/or 2D cultures and/or 3D cultures of healthy adult, healthy fetal/infant, undesired adult, undesired fetal/infant or progenitor cells like e.g. stem cell or cells derived from the same precursor lineage.

56. The cartilage array of claim 49, wherein the polynucleotide probes have a length of at least 10 nucleotides, preferably at least 25 nucleotides.

57. The cartilage array of claim 49, wherein the carrier is optionally attached to coated glass, nylon or any other material.

58. The cartilage array of claim 49, wherein at least part of the cartilage marker genes are selected from a subgroup of the 467 genes listed in the description, said subgroup consisting of the most tissue specific 200 genes.

59. The cartilage array of claim 49, which can be used within clinical applications as a diagnostic tool in order to assess patient biopsy/cell samples for targeted in vitro cell culture treatment/performance when performing a cell or tissue based therapy.

60. The cartilage array of claim 49, which can be used within clinical applications as a diagnostic tool in order to asses patient biopsy/cell samples and to decide on subsequent therapeutic approach which maybe a tissue engineered therapy, a cell therapy only, or even a traditional surgical approach only.

61. The cartilage array of claim 49, which can be used as a quality control tool in order to assess the quality of human biopsy/cell samples prior performing the cellular expansion in case of cell therapy and/or prior performing the differentiation/tissue formation in case of a tissue engineered therapy.

62. The cartilage array of claim 49, which can be used as a quality control tool in order to assess the quality of the final implant prior and/or after product release, said implant being proliferated cells in case of a cell therapy or tissue engineered cartilage in case of a tissue engineered therapeutic approach.

63. A kit for use in a hybridization assay comprising a cartilage array of claim 49.

64. The kit of claim 63, wherein said kit further comprises reagents for generating a labeled target polynucleotide sample, a hybridization buffer and a wash medium.

65. Use of a cartilage array of claim 49, for in vitro diagnostic of mammals, in particular humans.

66. Use of a kit of claim 63, for in vitro diagnostic of mammals, in particular humans.
